(19) United States
(12) Patent Application Publication
(10) Pub. No.: US 2024/0159779 A1
(43) Pub. Date: May 16, 2024

(54) VAGINAL LIPID PROFILES PREDICTIVE OF SUCCESSFUL OR UNSUCCESSFUL PREGNANCY IN A BOVINE SURROGATE DAM FOLLOWING EMBRYO TRANSFER

(71) Applicant: VYTELLE LLC, Lenexa, KS (US)

(72) Inventors: Bruno Valente Sanches, Southlake, TX (US); Tasha Gruhot, Hawarden, IA (US)

(21) Appl. No.: 18/508,448

(22) Filed: Nov. 14, 2023

Related U.S. Application Data
(60) Provisional application No. 63/383,643, filed on Nov. 14, 2022.

(51) Int. Cl. G01N 33/92

(52) U.S. Cl. CPC G01N 33/92; G01N 2405/04; G01N 2405/08; G01N 2800/36

(57) ABSTRACT

The present disclosure provides, inter alia, methods for assessing suitability of a bovine animal as an effective surrogate dam for embryo implantation and maintenance of pregnancy, which methods include obtaining a lipidome profile for a bovine animal, quantifying the ratios of individual lipids, in particular phospholipids, and determining whether the bovine animal exhibits a lipid ratio profile that is predictive of successful embryo implantation and, thereby, assessing the likelihood of a successful in vitro fertilization (IVF) derived embryo implantation, and pregnancy. In further aspects, the present disclosure provides methods for generating a lipidome database. Also provided are compositions and kits for use in those methods to obtain and quantify the lipidome profile of a bovine animal.

### CROSS REFERENCE TO RELATED APPLICATIONS

This application claims priority under 35 U.S.C. § 119 to Provisional Application U.S. Ser. No. 63/383,643, filed Nov. 14, 2022, the entirety of which is herein incorporated by reference.

### BACKGROUND OF THE DISCLOSURE

### Technical Field

The present disclosure relates, generally, to diagnostic technologies and methodologies for assessing the fertility of a candidate bovine surrogate dam and for improving the success rate of both embryo implantation and maintenance of pregnancy to full term.

### Description of the Related Art

In vitro fertilization (IVF) and embryo transfer techniques are commonly used in bovine calf production. IVF has some advantages over other production methods due to the frequency with which IVF aspirations can be performed and the ability to increase genetic diversity. For example, donor cow IVF aspirations can be performed every two weeks and semen from several different bulls can be used to fertilize harvested oocytes to produce a large number of offspring from a single cow. While traditional in vivo embryo flushing techniques can produce about five or six embryos per collection every sixty days, IVF collections can produce about 20 oocytes per aspiration every two weeks, of which about 30% develop into viable embryos. In some circumstances, IVF can be used to produce over 50 calves from one cow in a single year. Furthermore, pregnant donor cows can still be used for oocyte collection until about day 100 to day 120 of pregnancy, making it possible to produce embryos while still producing natural offspring from a high value cow.

There are, however, some disadvantages with using IVF techniques for ruminant offspring production. The IVF procedure can be expensive and the follicle aspiration method used to collect oocytes is an invasive procedure that requires a skilled technician. Furthermore, even when performed properly, freshly transferred IVF embryos result in an average pregnancy rate of about 50% for well managed recipient bovine surrogate dams. Of the transfers that result in pregnancy, between about 6% to about 16% of pregnancies are lost.

Existing methodologies for identifying a surrogate dam that is a suitable candidate for embryo transfer is the presence of a Corpus Luteum at time of transfer. Thus, despite recent advancements in IVF techniques, there remains an unmet need in the art for improved technologies and methodologies for maximizing the efficiency of offspring production, including diagnostic technologies and methodologies for assessing the fertility of a candidate recipient bovine surrogate dam and for improving the success rate of both embryo implantation and maintenance of pregnancy to full term.

### SUMMARY OF THE DISCLOSURE

The present disclosure fulfills unmet needs in the art by providing methods and associated diagnostic test kits for determining the suitability of candidate bovine animals to serve as surrogate dams as recipients for in vitro fertilization (IVF), including their ability or inability to establish a successful embryo implantation and to maintain a successful pregnancy. The methods and diagnostic test kits disclosed herein employ of vaginal lipid profiles from a bovine surrogate dam that are predictive of successful or unsuccessful embryo implantation and maintenance of pregnancy, which methods and diagnostic test kits overcome limitations in the art by permitting the identification of a suitable surrogate dam for embryo transfer at the time of or prior to the day of embryo transfer. The methods and diagnostic test kits disclosed herein can increase the selection accuracy of candidate surrogate dams and result in higher overall pregnancy rates.

Within certain embodiments, provided herein are methods for predicting in a candidate bovine surrogate dam an ability or inability to establish a successful embryo implantation and pregnancy following embryo transfer. In certain aspects, these methods comprise (1) obtaining vaginal fluids from a candidate bovine surrogate dam, wherein said vaginal fluids comprises a vaginal lipidome and (2) performing lipidome analysis on the vaginal fluids to obtain a vaginal lipid profile comprising ratios of two or more vaginal lipids and/or levels of individual vaginal lipids. According to these embodiments, increased or decreased vaginal lipid levels and/or ratios of lipids in a candidate bovine surrogate dam are predictive of a candidate bovine surrogate dam possessing a suitable or an unsuitable environment for achieving a successful embryo implantation and maintaining a successful pregnancy.

Within other embodiments, provided herein are methods for generating a lipidome database for identifying a bovine surrogate dam for embryo transfer. In certain aspects, these methods comprise (1) collecting vaginal fluids individually from a population of bovine animals, wherein the vaginal fluids comprise a lipidome profile of each bovine animal of the population, (2) performing lipidome profile analysis on the vaginal fluids to obtain a vaginal lipid profile comprising vaginal lipid levels and/or ratios of lipids, wherein the increased or decreased lipid ratios and/or levels in the vaginal lipid profile are predictive of a bovine surrogate dam possessing a suitable or an unsuitable environment for achieving a successful embryo implantation and maintaining a successful pregnancy, and (4) identifying an association of vaginal lipid levels and/or ratios of lipids in the vaginal lipidome profile with an increased probability of a successful embryo implantation and pregnancy following embryo delivery.

Within further embodiments, provided herein are methods for improving the success rate of in vitro fertilization embryo transfer in bovine animals serving as surrogate dams. In certain aspects, these methods comprise (1) collecting vaginal fluid from each of the bovine animals, wherein the vaginal fluid comprises a lipidome profile of each bovine animal; (2) performing lipidome profile analysis on the vaginal fluids to obtain a vaginal lipid profile comprising vaginal lipid levels and/or ratios of lipids, wherein an increased or decreased vaginal lipid ratio and/or level in the vaginal lipid profile is predictive of bovine animals possessing or not possessing an environment for achieving a successful embryo implantation and pregnancy following in vitro fertilization and therefore being suitable or unsuitable bovine animals for serving as surrogate dams; and (3) administering an intervention to one or more of the bovine surrogate dams, wherein the intervention is effective to provide or augment in a recipient bovine surrogate dam a lipidome profile that is associated with a high likelihood of successful in vitro fertilization, including reliable embryo implantation and maintenance of pregnancy.

Within still further embodiments, provided herein are diagnostic test kits for use in the identification of bovine surrogate dams having a high and/or a low probability of establishing a successful embryo implantation and maintaining pregnancy following embryo transfer via in vitro fertilization. In certain aspects, diagnostic test kits comprise: (1) one or more analytical tools for determining a vaginal lipid profile comprising vaginal lipid levels and/or ratios of lipids from a bovine animal; (2) a transmitter for communicating with and/or connecting to a database of one or more vaginal lipid ratios and/or levels of a bovine vaginal fluid; (3) a device for comparing the vaginal lipid ratios and/or levels of the vaginal fluid from candidate bovine surrogate dams with vaginal lipid ratios and/or levels in a database of vaginal lipidomes to identify a bovine surrogate dam having a vaginal lipid ratio or level that is predictive of a high or a low success rate of embryo implantation and maintaining pregnancy after receiving an in vitro fertilization derived embryo; and, optionally, (4) instructions for the use of the diagnostic test kits. Diagnostic test kits according to these embodiments may employ mass spectrometry techniques, including but not limited to MRM (multiple reaction monitoring) mass spectroscopy, or positive-negative ion mode mass spectroscopy, and instruments to allow field or onsite collection of vaginal fluids and subsequent lipidome profile analysis to establish lipid profiles and ratios that convey a positive or negative relationship to the maternal recognition of pregnancy and intrauterine embryo implantation.

These and other related aspects of the present disclosure will be better understood in light of the following drawings and detailed description, which exemplify various embodiments, and which are presented for illustration, not limitation.

### BRIEF DESCRIPTION OF THE DRAWINGS

FIGS. 1-7 are Receiver Operating Characteristic (ROC) curves obtained for individual vaginal lipids and lipid ratios from the vaginal lipidome of a bovine animal (i.e., a surrogate dam) prior to embryo transfer. Selected lipids or lipid ratios used in the displayed ROCs individually had an area under the curve (AUC) of at least 0.7 to be applied to the combined ROC curve analysis. ROC curves of greater than 0.7 when the individual lipids and lipid ratios were combined are displayed. An AUC of greater than 0.7 indicates the selected lipids/lipid ratios are accurately distinguishing between pregnancy and non-pregnant dairy females.

FIG. 1 is a Receiver Operating Characteristic (ROC) curve of a combination of 5 lipid ratios and individual lipids used in combination (FIG. 1A). (FIG. 1B) is the list of the lipids and lipid ratios included in the ROC analysis presented with their individual AUC.

FIG. 2 is a Receiver Operating Characteristic (ROC) curve of a combination of 29 lipid ratios and individual lipids used in combination (FIG. 2A). (FIG. 2B) is the list of the lipids and lipid ratios included in the ROC analysis presented with their individual AUC.

FIG. 3 is a Receiver Operating Characteristic (ROC) curve of a combination of 6 lipid ratios and individual lipids used in combination (FIG. 3A). (FIG. 3B) is the list of the lipids and lipid ratios included in the ROC analysis presented with their individual AUC.

FIG. 4 is a Receiver Operating Characteristic (ROC) curve of a combination of 2 lipid ratios and individual lipids used in combination (FIG. 4A). (FIG. 4B) is the list of the lipids and lipid ratios included in the ROC analysis presented with their individual AUC.

FIG. 5 is a Receiver Operating Characteristic (ROC) curve of a combination of 20 lipid ratios and individual lipids used in combination (FIG. 5A). (FIG. 5B) is the list of the lipids and lipid ratios included in the ROC analysis presented with their individual AUC.

FIG. 6 is a Receiver Operating Characteristic (ROC) curve of a combination of 6 lipid ratios and individual lipids used in combination (FIG. 6A). (FIG. 6B) is the list of the lipids and lipid ratios included in the ROC analysis presented with their individual AUC.

FIG. 7 is a Receiver Operating Characteristic (ROC) curve of a combination of 6 lipid ratios and individual lipids used in combination (FIG. 7A). (FIG. 7B) is the list of the lipids and lipid ratios included in the ROC analysis presented with their individual AUC.

FIGS. 8-11 are Receiver Operating Characteristic (ROC) curves obtained for individual vaginal lipids and lipid ratios from the vaginal lipidome of a bovine animal (i.e., a surrogate dam) prior to embryo transfer. Selected lipids or lipid ratios used in the displayed ROCs individually had an area under the curve (AUC) of at least 0.7 to be applied to the combined ROC curve analysis. ROC curves of greater than 0.7 when the individual lipids and lipid ratios were combined are displayed. An AUC of greater than 0.7 indicates the selected lipids/lipid ratios are accurately distinguishing between pregnancy and non-pregnant beef females.

FIG. 8 is a Receiver Operating Characteristic (ROC) curve of a combination of 18 lipid ratios and individual lipids used in combination (FIG. 8A). (FIG. 8B) is the list of the lipids and lipid ratios included in the ROC analysis presented with their individual AUC.

FIG. 9 is a Receiver Operating Characteristic (ROC) curve of a combination of 13 lipid ratios and individual lipids used in combination (FIG. 9A). (FIG. 9B) is the list of the lipids and lipid ratios included in the ROC analysis presented with their individual AUC.

FIG. 10 is a Receiver Operating Characteristic (ROC) curve of a combination of 9 lipid ratios and individual lipids used in combination (FIG. 10A). (FIG. 10B) is the list of the lipids and lipid ratios included in the ROC analysis presented with their individual AUC.

FIG. 11 is a Receiver Operating Characteristic (ROC) curve of a combination of 6 lipid ratios and individual lipids used in combination (FIG. 11A). (FIG. 11B) is the list of the lipids and lipid ratios included in the ROC analysis presented with their individual AUC.

FIGS. 12-107 are individual lipids that had significant differences (p<0.05) in concentration between pregnant and non-pregnant dairy bovine females based on a statistical t-test. Presented are the box plots displaying the raw concentration (A) and normalized concertation (B).

FIG. 12 are the boxplots (FIG. 12A, Original concentration; FIG. 12B, Normalized concentration) displaying the significant difference (p<0.05) in the concentration (and normalized concentration) of the lipid Cer(d14:1/24:0(20H)) indicating a significantly higher concentration in the non-pregnant females.

FIG. 13 are the boxplots (FIG. 13A, Original concentration; FIG. 13B, Normalized concentration) displaying the significant difference (p<0.05) in the concentration (and normalized concentration) of the lipid TAG(50:2)_FA 16:0 indicating a significantly higher concentration in the non-pregnant females.

FIG. 14 are the boxplots (FIG. 14A, Original concentration; FIG. 14B, Normalized concentration) displaying the significant difference (p<0.05) in the concentration (and normalized concentration) of the lipid Cer(d18:1/16:0) indicating a significantly higher concentration in the pregnant females.

FIG. 15 are the boxplots (FIG. 15A, Original concentration; FIG. 15B, Normalized concentration) displaying the significant difference (p<0.05) in the concentration (and normalized concentration) of the lipid TAG(50:1)_FA 18:1 indicating a significantly higher concentration in the non-pregnant females.

FIG. 16 are the boxplots (FIG. 16A, Original concentration; FIG. 16B, Normalized concentration) displaying the significant difference (p<0.05) in the concentration (and normalized concentration) of the lipid TAG(52:1)_FA 18:1 indicating a significantly higher concentration in the non-pregnant females.

FIG. 17 are the boxplots (FIG. 17A, Original concentration; FIG. 17B, Normalized concentration) displaying the significant difference (p<0.05) in the concentration (and normalized concentration) of the lipid Lyso PC(19:2) indicating a significantly higher concentration in the non-pregnant females.

FIG. 18 are the boxplots (FIG. 18A, Original concentration; FIG. 18B, Normalized concentration) displaying the significant difference (p<0.05) in the concentration (and normalized concentration) of the lipid Lyso PC(17:2) indicating a significantly higher concentration in the non-pregnant females.

FIG. 19 are the boxplots (FIG. 19A, Original concentration; FIG. 19B, Normalized concentration) displaying the significant difference (p<0.05) in the concentration (and normalized concentration) of the lipid Lyso PC(17:1) indicating a significantly higher concentration in the non-pregnant females.

FIG. 20 are the boxplots (FIG. 20A, Original concentration; FIG. 20B, Normalized concentration) displaying the significant difference (p<0.05) in the concentration (and normalized concentration) of the lipid Lyso PC(17:0) indicating a significantly higher concentration in the non-pregnant females.

FIG. 21 are the boxplots (FIG. 21A, Original concentration; FIG. 21B, Normalized concentration) displaying the significant difference (p<0.05) in the concentration (and normalized concentration) of the lipid Lyso PC(16:1) indicating a significantly higher concentration in the non-pregnant females.

FIG. 22 are the boxplots (FIG. 22A, Original concentration; FIG. 22B, Normalized concentration) displaying the significant difference (p<0.05) in the concentration (and normalized concentration) of the lipid SM(d18:2/20:1) indicating a significantly higher concentration in the non-pregnant females.

FIG. 23 are the boxplots (FIG. 23A, Original concentration; FIG. 23B, Normalized concentration) displaying the significant difference (p<0.05) in the concentration (and normalized concentration) of the lipid SM(d18:0/26:0) indicating a significantly higher concentration in the non-pregnant females.

FIG. 24 are the boxplots (FIG. 24A, Original concentration; FIG. 24B, Normalized concentration) displaying the significant difference (p<0.05) in the concentration (and normalized concentration) of the lipid SM(d16:1/24:0) indicating a significantly higher concentration in the pregnant females.

FIG. 25 are the boxplots (FIG. 25A, Original concentration; FIG. 25B, Normalized concentration) displaying the significant difference (p<0.05) in the concentration (and normalized concentration) of the lipid SM(d18:1/26:0) indicating a significantly higher concentration in the non-pregnant females.

FIG. 26 are the boxplots (FIG. 26A, Original concentration; FIG. 26B, Normalized concentration) displaying the significant difference (p<0.05) in the concentration (and normalized concentration) of the lipid SM(d18:0/14:0) indicating a significantly higher concentration in the non-pregnant females.

FIG. 27 are the boxplots (FIG. 27A, Original concentration; FIG. 27B, Normalized concentration) displaying the significant difference (p<0.05) in the concentration (and normalized concentration) of the lipid SM(d18:1/16:0) indicating a significantly higher concentration in the pregnant females.

FIG. 28 are the boxplots (FIG. 28A, Original concentration; FIG. 28B, Normalized concentration) displaying the significant difference (p<0.05) in the concentration (and normalized concentration) of the lipid PEp(36:5) indicating a significantly higher concentration in the non-pregnant females.

FIG. 29 are the boxplots (FIG. 29A, Original concentration; FIG. 29B, Normalized concentration) displaying the significant difference (p<0.05) in the concentration (and normalized concentration) of the lipid PEo(40:4) indicating a significantly higher concentration in the non-pregnant females.

FIG. 30 are the boxplots (FIG. 30A, Original concentration; FIG. 30B, Normalized concentration) displaying the significant difference (p<0.05) in the concentration (and normalized concentration) of the lipid PEo(38:4) indicating a significantly higher concentration in the non-pregnant females.

FIG. 31 are the boxplots (FIG. 31A, Original concentration; FIG. 31B, Normalized concentration) displaying the significant difference (p<0.05) in the concentration (and normalized concentration) of the lipid PEp(32:1) indicating a significantly higher concentration in the non-pregnant females.

FIG. 32 are the boxplots (FIG. 32A, Original concentration; FIG. 32B, Normalized concentration) displaying the significant difference (p<0.05) in the concentration (and normalized concentration) of the lipid PEo(40:6) indicating a significantly higher concentration in the non-pregnant females.

FIG. 33 are the boxplots (FIG. 33A, Original concentration; FIG. 33B, Normalized concentration) displaying the significant difference (p<0.05) in the concentration (and normalized concentration) of the lipid PEo(38:6) indicating a significantly higher concentration in the non-pregnant females.

FIG. 34 are the boxplots (FIG. 34A, Original concentration; FIG. 34B, Normalized concentration) displaying the significant difference (p<0.05) in the concentration (and normalized concentration) of the lipid PEo(36:5) indicating a significantly higher concentration in the non-pregnant females.

FIG. 35 are the boxplots (FIG. 35A, Original concentration; FIG. 35B, Normalized concentration) displaying the significant difference (p<0.05) in the concentration (and normalized concentration) of the lipid PEo(32:5) indicating a significantly higher concentration in the non-pregnant females.

FIG. 36 are the boxplots (FIG. 36A, Original concentration; FIG. 36B, Normalized concentration) displaying the significant difference (p<0.05) in the concentration (and normalized concentration) of the lipid PEo(34:0) indicating a significantly higher concentration in the non-pregnant females.

FIG. 37 are the boxplots (FIG. 37A, Original concentration; FIG. 37B, Normalized concentration) displaying the significant difference (p<0.05) in the concentration (and normalized concentration) of the lipid PEo(34:3) indicating a significantly higher concentration in the non-pregnant females.

FIG. 38 are the boxplots (FIG. 38A, Original concentration; FIG. 38B, Normalized concentration) displaying the significant difference (p<0.05) in the concentration (and normalized concentration) of the lipid PEo(32:1) indicating a significantly higher concentration in the non-pregnant females.

FIG. 39 are the boxplots (FIG. 39A, Original concentration; FIG. 39B, Normalized concentration) displaying the significant difference (p<0.05) in the concentration (and normalized concentration) of the lipid PEo(28:0) indicating a significantly higher concentration in the non-pregnant females.

FIG. 40 are the boxplots (FIG. 40A, Original concentration; FIG. 40B, Normalized concentration) displaying the significant difference (p<0.05) in the concentration (and normalized concentration) of the lipid PE(42:10), PEp(42:2) indicating a significantly higher concentration in the non-pregnant females.

FIG. 41 are the boxplots (FIG. 41A, Original concentration; FIG. 41B, Normalized concentration) displaying the significant difference (p<0.05) in the concentration (and normalized concentration) of the lipid PE(42:4) indicating a significantly higher concentration in the non-pregnant females.

FIG. 42 are the boxplots (FIG. 42A, Original concentration; FIG. 42B, Normalized concentration) displaying the significant difference (p<0.05) in the concentration (and normalized concentration) of the lipid PE(40:9), PEo(40:2) indicating a significantly higher concentration in the non-pregnant females.

FIG. 43 are the boxplots (FIG. 43A, Original concentration; FIG. 43B, Normalized concentration) displaying the significant difference (p<0.05) in the concentration (and normalized concentration) of the lipid PE(42:6) indicating a significantly higher concentration in the non-pregnant females.

FIG. 44 are the boxplots (FIG. 44A, Original concentration; FIG. 44B, Normalized concentration) displaying the significant difference (p<0.05) in the concentration (and normalized concentration) of the lipid PE(42:2) indicating a significantly higher concentration in the non-pregnant females.

FIG. 45 are the boxplots (FIG. 45A, Original concentration; FIG. 45B, Normalized concentration) displaying the significant difference (p<0.05) in the concentration (and normalized concentration) of the lipid PE(42:3) indicating a significantly higher concentration in the non-pregnant females.

FIG. 46 are the boxplots (FIG. 46A, Original concentration; FIG. 46B, Normalized concentration) displaying the significant difference (p<0.05) in the concentration (and normalized concentration) of the lipid PE(40:8), PEo(40:1) indicating a significantly higher concentration in the non-pregnant females.

FIG. 47 are the boxplots (FIG. 47A, Original concentration; FIG. 47B, Normalized concentration) displaying the significant difference (p<0.05) in the concentration (and normalized concentration) of the lipid PE(38:8), PEo(38:1) indicating a significantly higher concentration in the non-pregnant females.

FIG. 48 are the boxplots (FIG. 48A, Original concentration; FIG. 48B, Normalized concentration) displaying the significant difference (p<0.05) in the concentration (and normalized concentration) of the lipid PE(40:7), PEo(40:0) indicating a significantly higher concentration in the non-pregnant females.

FIG. 49 are the boxplots (FIG. 49A, Original concentration; FIG. 49B, Normalized concentration) displaying the significant difference (p<0.05) in the concentration (and normalized concentration) of the lipid PE(38:7), PEo(38:0) indicating a significantly higher concentration in the non-pregnant females.

FIG. 50 are the boxplots (FIG. 50A, Original concentration; FIG. 50B, Normalized concentration) displaying the significant difference (p<0.05) in the concentration (and normalized concentration) of the lipid PE(40:1) indicating a significantly higher concentration in the non-pregnant females.

FIG. 51 are the boxplots (FIG. 51A, Original concentration; FIG. 51B, Normalized concentration) displaying the significant difference (p<0.05) in the concentration (and normalized concentration) of the lipid PE(40:6) indicating a significantly higher concentration in the non-pregnant females.

FIG. 52 are the boxplots (FIG. 52A, Original concentration; FIG. 52B, Normalized concentration) displaying the significant difference (p<0.05) in the concentration (and normalized concentration) of the lipid PE(38:0), PE(40:6) indicating a significantly higher concentration in the non-pregnant females.

FIG. 53 are the boxplots (FIG. 53A, Original concentration; FIG. 53B, Normalized concentration) displaying the significant difference (p<0.05) in the concentration (and normalized concentration) of the lipid PE(34:3) indicating a significantly higher concentration in the non-pregnant females.

FIG. 54 are the boxplots (FIG. 54A, Original concentration; FIG. 54B, Normalized concentration) displaying the significant difference (p<0.05) in the concentration (and normalized concentration) of the lipid PE(36:4) indicating a significantly higher concentration in the non-pregnant females.

FIG. 55 are the boxplots (FIG. 55A, Original concentration; FIG. 55B, Normalized concentration) displaying the significant difference (p<0.05) in the concentration (and normalized concentration) of the lipid PE(36:7), PEo(36:0) indicating a significantly higher concentration in the non-pregnant females.

FIG. 56 are the boxplots (FIG. 56A, Original concentration; FIG. 56B, Normalized concentration) displaying the significant difference (p<0.05) in the concentration (and normalized concentration) of the lipid PE(34:4) indicating a significantly higher concentration in the non-pregnant females.

FIG. 57 are the boxplots (FIG. 57A, Original concentration; FIG. 57B, Normalized concentration) displaying the significant difference (p<0.05) in the concentration (and normalized concentration) of the lipid PE(36:1) indicating a significantly higher concentration in the pregnant females.

FIG. 58 are the boxplots (FIG. 58A, Original concentration; FIG. 58B, Normalized concentration) displaying the significant difference (p<0.05) in the concentration (and normalized concentration) of the lipid PE(32:2) indicating a significantly higher concentration in the non-pregnant females.

FIG. 59 are the boxplots (FIG. 59A, Original concentration; FIG. 59B, Normalized concentration) displaying the significant difference (p<0.05) in the concentration (and normalized concentration) of the lipid PE(18:0), Lyso PE(18:0) indicating a significantly higher concentration in the non-pregnant females.

FIG. 60 are the boxplots (FIG. 60A, Original concentration; FIG. 60B, Normalized concentration) displaying the significant difference (p<0.05) in the concentration (and normalized concentration) of the lipid PE(32:0) indicating a significantly higher concentration in the non-pregnant females.

FIG. 61 are the boxplots (FIG. 61A, Original concentration; FIG. 61B, Normalized concentration) displaying the significant difference (p<0.05) in the concentration (and normalized concentration) of the lipid PE(32:1) indicating a significantly higher concentration in the non-pregnant females.

FIG. 62 are the boxplots (FIG. 62A, Original concentration; FIG. 62B, Normalized concentration) displaying the significant difference (p<0.05) in the concentration (and normalized concentration) of the lipid PE(22:2), Lyso PE(22:2) indicating a significantly higher concentration in the non-pregnant females.

FIG. 63 are the boxplots (FIG. 63A, Original concentration; FIG. 63B, Normalized concentration) displaying the significant difference (p<0.05) in the concentration (and normalized concentration) of the lipid PE(30:1) indicating a significantly higher concentration in the non-pregnant females.

FIG. 64 are the boxplots (FIG. 64A, Original concentration; FIG. 64B, Normalized concentration) displaying the significant difference (p<0.05) in the concentration (and normalized concentration) of the lipid PCp(42:4) indicating a significantly higher concentration in the non-pregnant females.

FIG. 65 are the boxplots (FIG. 65A, Original concentration; FIG. 65B, Normalized concentration) displaying the significant difference (p<0.05) in the concentration (and normalized concentration) of the lipid PCo(42:6) indicating a significantly higher concentration in the non-pregnant females.

FIG. [65]66 are the boxplots (FIG. 66A, Original concentration; FIG. 66B, Normalized concentration) displaying the significant difference (p<0.05) in the concentration (and normalized concentration) of the lipid PCo(40:6) indicating a significantly higher concentration in the non-pregnant females.

FIG. [66]67 are the boxplots (FIG. 67A, Original concentration; FIG. 67B, Normalized concentration) displaying the significant difference (p<0.05) in the concentration (and normalized concentration) of the lipid PCo(40:4) indicating a significantly higher concentration in the non-pregnant females.

FIG. [67]68 are the boxplots (FIG. 68A, Original concentration; FIG. 68B, Normalized concentration) displaying the significant difference (p<0.05) in the concentration (and normalized concentration) of the lipid PCo(40:5) indicating a significantly higher concentration in the non-pregnant females.

FIG. [68]69 are the boxplots (FIG. 69A, Original concentration; FIG. 69B, Normalized concentration) displaying the significant difference (p<0.05) in the concentration (and normalized concentration) of the lipid PCo(38:4) indicating a significantly higher concentration in the non-pregnant females.

FIG. [69]70 are the boxplots (FIG. 70A, Original concentration; FIG. 70B, Normalized concentration) displaying the significant difference (p<0.05) in the concentration (and normalized concentration) of the lipid PCo(38:3) indicating a significantly higher concentration in the non-pregnant females.

FIG. [70]71 are the boxplots (FIG. 71A, Original concentration; FIG. 71B, Normalized concentration) displaying the significant difference (p<0.05) in the concentration (and normalized concentration) of the lipid PCo(32:3) indicating a significantly higher concentration in the non-pregnant females.

FIG. [71]72 are the boxplots (FIG. 72A, Original concentration; FIG. 72B, Normalized concentration) displaying the significant difference (p<0.05) in the concentration (and normalized concentration) of the lipid PCo(32:0) indicating a significantly higher concentration in the non-pregnant females.

FIG. [72]73 are the boxplots (FIG. 73A, Original concentration; FIG. 73B, Normalized concentration) displaying the significant difference (p<0.05) in the concentration (and normalized concentration) of the lipid PC(40:10), PCo(40:3) indicating a significantly higher concentration in the pregnant females.

FIG. [73]74 are the boxplots (FIG. 74A, Original concentration; FIG. 74B, Normalized concentration) displaying the significant difference (p<0.05) in the concentration (and normalized concentration) of the lipid PC(42:10), PCo(42:3) indicating a significantly higher concentration in the non-pregnant females.

FIG. [7]75 are the boxplots (FIG. 75A, Original concentration; FIG. 75B, Normalized concentration) displaying the significant difference (p<0.05) in the concentration (and normalized concentration) of the lipid PC(44:10), PCo(44:3) indicating a significantly higher concentration in the non-pregnant females.

FIG. [75]76 are the boxplots (FIG. 76A, Original concentration; FIG. 76B, Normalized concentration) displaying the significant difference (p<0.05) in the concentration (and normalized concentration) of the lipid PC(44:5) indicating a significantly higher concentration in the non-pregnant females.

FIG. [76]77 are the boxplots (FIG. 77A, Original concentration; FIG. 77B, Normalized concentration) displaying the significant difference (p<0.05) in the concentration (and normalized concentration) of the lipid PC(42:8), PCo(42:1) indicating a significantly higher concentration in the non-pregnant females.

FIG. [77]78 are the boxplots (FIG. 78A, Original concentration; FIG. 78B, Normalized concentration) displaying the significant difference (p<0.05) in the concentration (and normalized concentration) of the lipid PC(44:2) indicating a significantly higher concentration in the non-pregnant females.

FIG. [78]79 are the boxplots (FIG. 79A, Original concentration; FIG. 79B, Normalized concentration) displaying the significant difference (p<0.05) in the concentration (and normalized concentration) of the lipid PC(44:3) indicating a significantly higher concentration in the non-pregnant females.

FIG. [79]80 are the boxplots (FIG. 80A, Original concentration; FIG. 80B, Normalized concentration) displaying the significant difference (p<0.05) in the concentration (and normalized concentration) of the lipid PC(42:9), PCo(42:2 indicating a significantly higher concentration in the non-pregnant females.

FIG. [80]81 are the boxplots (FIG. 81A, Original concentration; FIG. 81B, Normalized concentration) displaying the significant difference (p<0.05) in the concentration (and normalized concentration) of the lipid PC(42:6) indicating a significantly higher concentration in the non-pregnant females.

FIG. [81]82 are the boxplots (FIG. 82A, Original concentration; FIG. 82B, Normalized concentration) displaying the significant difference (p<0.05) in the concentration (and normalized concentration) of the lipid PC(42:5) indicating a significantly higher concentration in the non-pregnant females.

FIG. [82]83 are the boxplots (FIG. 83A, Original concentration; FIG. 83B, Normalized concentration) displaying the significant difference (p<0.05) in the concentration (and normalized concentration) of the lipid PC(42:2) indicating a significantly higher concentration in the non-pregnant females.

FIG. [83]84 are the boxplots (FIG. 84A, Original concentration; FIG. 84B, Normalized concentration) displaying the significant difference (p<0.05) in the concentration (and normalized concentration) of the lipid PC(42:1) indicating a significantly higher concentration in the non-pregnant females.

FIG. [84]85 are the boxplots (FIG. 85A, Original concentration; FIG. 85B, Normalized concentration) displaying the significant difference (p<0.05) in the concentration (and normalized concentration) of the lipid PC(42:3) indicating a significantly higher concentration in the non-pregnant females.

FIG. [85]86 are the boxplots (FIG. 86A, Original concentration; FIG. 86B, Normalized concentration) displaying the significant difference (p<0.05) in the concentration (and normalized concentration) of the lipid PC(40:7), PCo(40:0) indicating a significantly higher concentration in the non-pregnant females.

FIG. [86]87 are the boxplots (FIG. 87A, Original concentration; FIG. 87B, Normalized concentration) displaying the significant difference (p<0.05) in the concentration (and normalized concentration) of the lipid PC(40:6) indicating a significantly higher concentration in the non-pregnant females.

FIG. [87]88 are the boxplots (FIG. 88A, Original concentration; FIG. 88B, Normalized concentration) displaying the significant difference (p<0.05) in the concentration (and normalized concentration) of the lipid PC(38:2) indicating a significantly higher concentration in the non-pregnant females.

FIG. [88]89 are the boxplots (FIG. 89A, Original concentration; FIG. 89B, Normalized concentration) displaying the significant difference (p<0.05) in the concentration (and normalized concentration) of the lipid PC(40:0), PCo(42:6) indicating a significantly higher concentration in the pregnant females.

FIG. [89]90 are the boxplots (FIG. 90A, Original concentration; FIG. 90B, Normalized concentration) displaying the significant difference (p<0.05) in the concentration (and normalized concentration) of the lipid PC(40:1) indicating a significantly higher concentration in the non-pregnant females.

FIG. [90]91 are the boxplots (FIG. 91A, Original concentration; FIG. 91B, Normalized concentration) displaying the significant difference (p<0.05) in the concentration (and normalized concentration) of the lipid PC(38:7), PCo(38:0) indicating a significantly higher concentration in the non-pregnant females.

FIG. [91]92 are the boxplots (FIG. 92A, Original concentration; FIG. 92B, Normalized concentration) displaying the significant difference (p<0.05) in the concentration (and normalized concentration) of the lipid PC(38:5) indicating a significantly higher concentration in the non-pregnant females.

FIG. [92]93 are the boxplots (FIG. 93A, Original concentration; FIG. 93B, Normalized concentration) displaying the significant difference (p<0.05) in the concentration (and normalized concentration) of the lipid PC(36:5) indicating a significantly higher concentration in the non-pregnant females.

FIG. [93]94 are the boxplots (FIG. 94A, Original concentration; FIG. 94B, Normalized concentration) displaying the significant difference (p<0.05) in the concentration (and normalized concentration) of the lipid PC(34:3) indicating a significantly higher concentration in the non-pregnant females.

FIG. [94]95 are the boxplots (FIG. 95A, Original concentration; FIG. 95B, Normalized concentration) displaying the significant difference (p<0.05) in the concentration (and normalized concentration) of the lipid PC(32:2) indicating a significantly higher concentration in the non-pregnant females.

FIG. [95]96 are the boxplots (FIG. 96A, Original concentration; FIG. 96B, Normalized concentration) displaying the significant difference (p<0.05) in the concentration (and normalized concentration) of the lipid Lyso PC(20:4) indicating a significantly higher concentration in the non-pregnant females.

FIG. [96]97 are the boxplots (FIG. 97A, Original concentration; FIG. 97B, Normalized concentration) displaying the significant difference (p<0.05) in the concentration (and normalized concentration) of the lipid Lyso PC(22:2) indicating a significantly higher concentration in the non-pregnant females.

FIG. [97]98 are the boxplots (FIG. 98A, Original concentration; FIG. 98B, Normalized concentration) displaying the significant difference (p<0.05) in the concentration (and normalized concentration) of the lipid Lyso PC(30:2) indicating a significantly higher concentration in the non-pregnant females.

FIG. [98]99 are the boxplots (FIG. 99A, Original concentration; FIG. 99B, Normalized concentration) displaying the significant difference (p<0.05) in the concentration (and normalized concentration) of the lipid PSp(16:0) indicating a significantly higher concentration in the non-pregnant females.

FIG. [99]100 are the boxplots (FIG. 100A, Original concentration; FIG. 100B, Normalized concentration) displaying the significant difference (p<0.05) in the concentration (and normalized concentration) of the lipid PS(32:2) indicating a significantly higher concentration in the non-pregnant females.

FIG. [100]101 are the boxplots (FIG. 101A, Original concentration; FIG. 101B, Normalized concentration) displaying the significant difference (p<0.05) in the concentration (and normalized concentration) of the lipid Plo(20:5) indicating a significantly higher concentration in the non-pregnant females.

FIG. [101]102 are the boxplots (FIG. 102A, Original concentration; FIG. 102B, Normalized concentration) displaying the significant difference (p<0.05) in the concentration (and normalized concentration) of the lipid Plo(36:2), Plp(36:1) indicating a significantly higher concentration in the non-pregnant females.

FIG. [102]103 are the boxplots (FIG. 103A, Original concentration; FIG. 103B, Normalized concentration) displaying the significant difference (p<0.05) in the concentration (and normalized concentration) of the lipid PI(36:8), Plo(36:1), Plp(36:0) indicating a significantly higher concentration in the non-pregnant females.

FIG. [103]104 are the boxplots (FIG. 104A, Original concentration; FIG. 104B, Normalized concentration) displaying the significant difference (p<0.05) in the concentration (and normalized concentration) of the lipid PI(36:4) indicating a significantly higher concentration in the non-pregnant females.

FIG. [104]105 are the boxplots (FIG. 105A, Original concentration; FIG. 105B, Normalized concentration) displaying the significant difference (p<0.05) in the concentration (and normalized concentration) of the lipid 20:5 Cholesteryl ester indicating a significantly higher concentration in the non-pregnant females.

FIG. [105]106 are the boxplots (FIG. 106A, Original concentration; FIG. 106B, Normalized concentration) displaying the significant difference (p<0.05) in the concentration (and normalized concentration) of the lipid 18:2 Cholesteryl ester indicating a significantly higher concentration in the non-pregnant females.

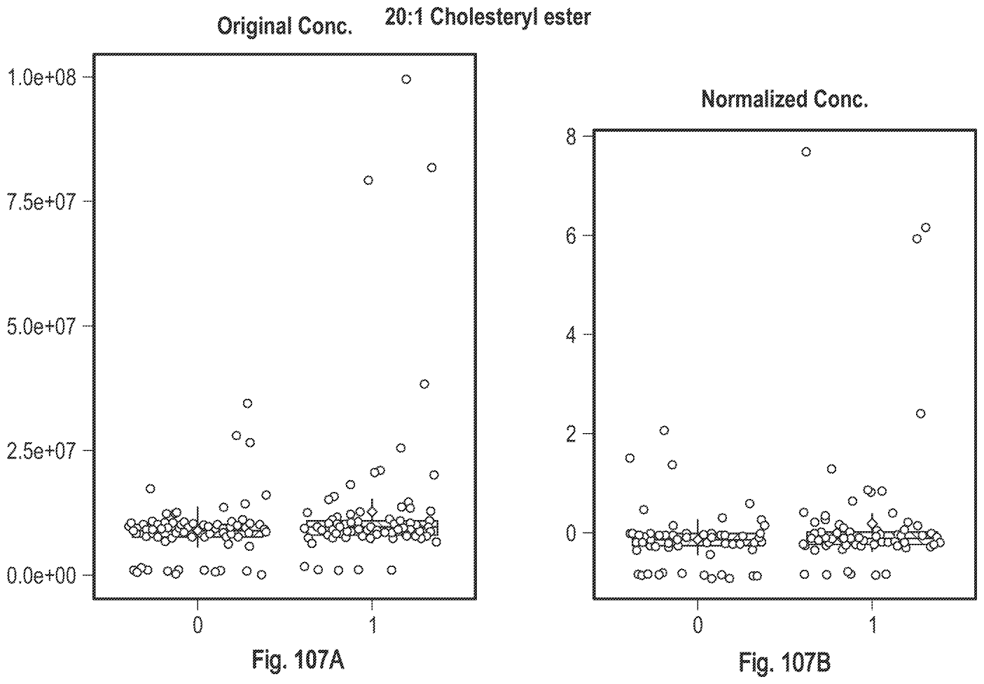

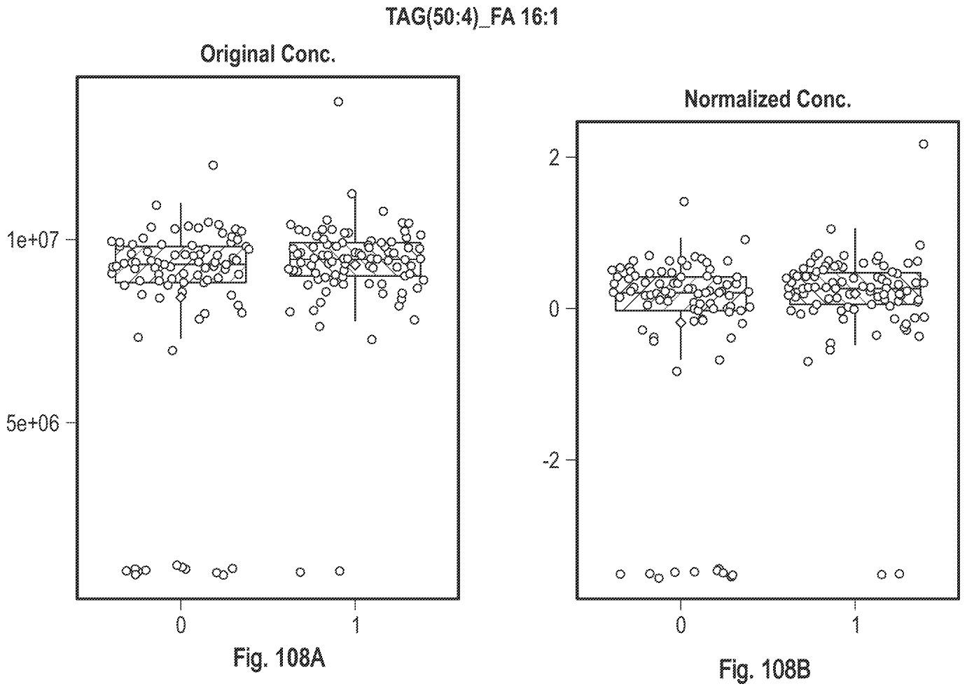

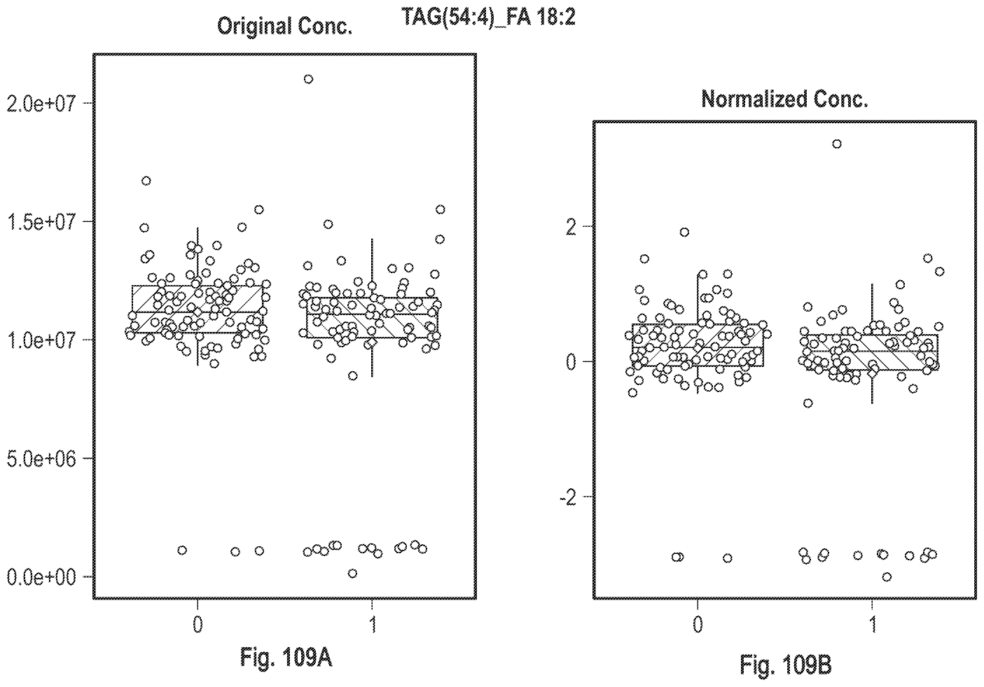

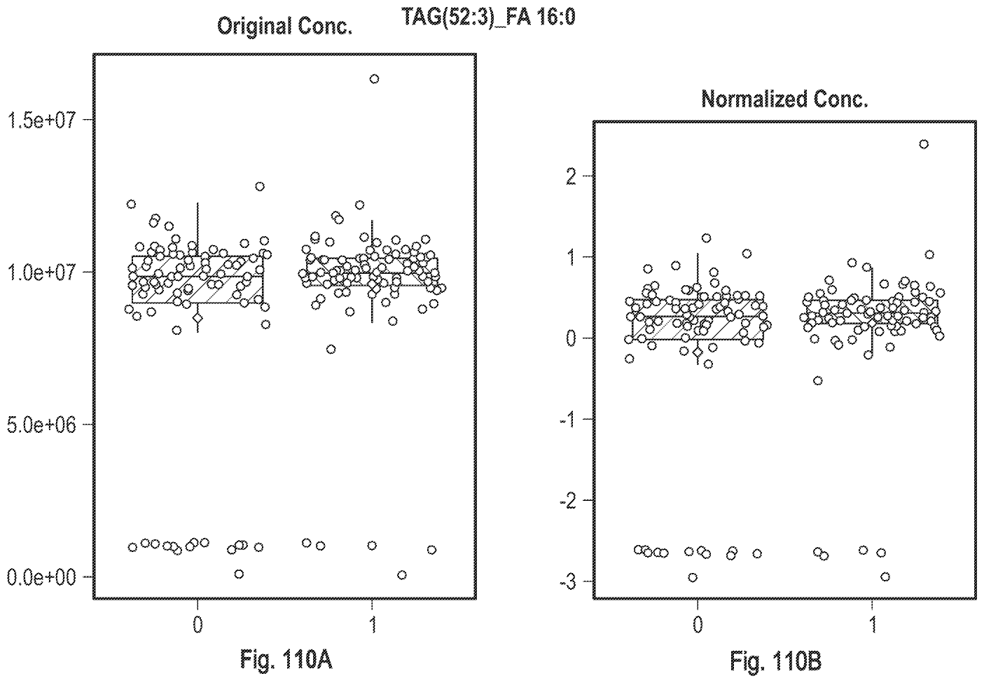

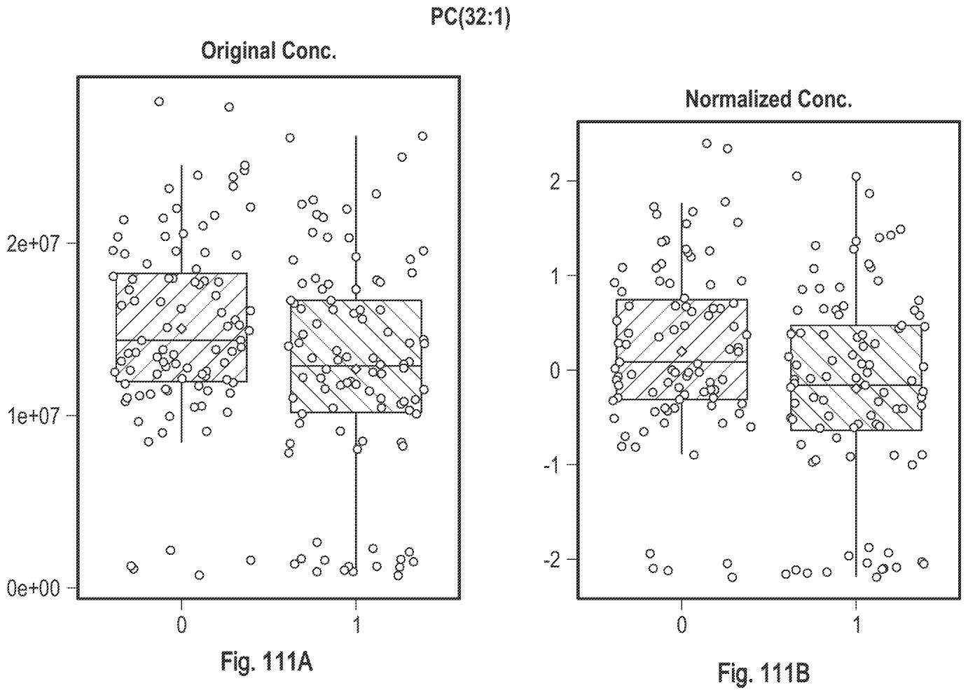

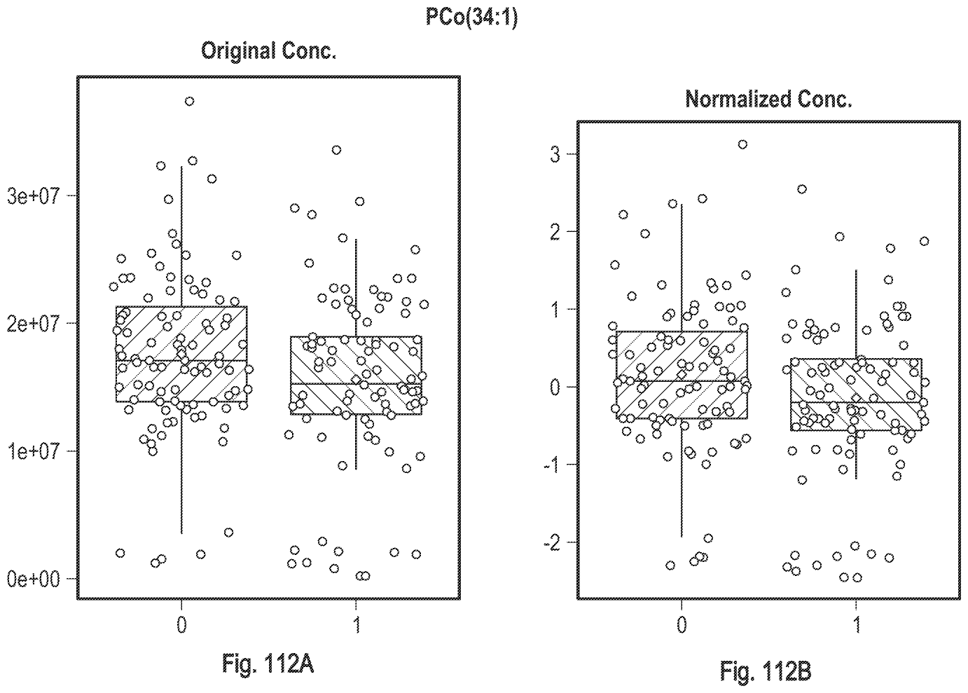

FIG. [106]107 are the boxplots (FIG. 107A, Original concentration; FIG. 107B, Normalized concentration) displaying the significant difference (p<0.05) in the concentration (and normalized concentration) of the lipid 20:1 Cholesteryl ester indicating a significantly higher concentration in the pregnant females.

FIGS. 108-112 are individual lipids that had significant differences (p<0.05) in concentration between pregnant and non-pregnant beef bovine females based on a statistical t-test. Presented are the box plots displaying the raw concentration and normalized concertation.

FIG. 108 are the boxplots (FIG. 108A, Original concentration; FIG. 108B, Normalized concentration) displaying the significant difference (p<0.05) in the concentration (and normalized concentration) of the lipid TAG(50:4)_FA 16:1 indicating a significantly higher concentration in the pregnant females.

FIG. 109 are the boxplots (FIG. 109A, Original concentration; FIG. 109B, Normalized concentration) displaying the significant difference (p<0.05) in the concentration (and normalized concentration) of the lipid TAG(54:4)_FA 18:2 indicating a significantly higher concentration in the non-pregnant females.

FIG. 110 are the boxplots (FIG. 110A, Original concentration; FIG. 110B, Normalized concentration) displaying the significant difference (p<0.05) in the concentration (and normalized concentration) of the lipid TAG(52:3)_FA 16:0 indicating a significantly higher concentration in the pregnant females.

FIG. 111 are the boxplots (FIG. 111A, Original concentration; FIG. 111B, Normalized concentration) displaying the significant difference (p<0.05) in the concentration (and normalized concentration) of the lipid PC(32:1) indicating a significantly higher concentration in the non-pregnant females.

FIG. 112 are the boxplots (FIG. 112A, Original concentration; FIG. 112B, Normalized concentration) displaying the significant difference (p<0.05) in the concentration (and normalized concentration) of the lipid PCo(34:1) indicating a significantly higher concentration in the non-pregnant females.

### DETAILED DESCRIPTION

The present disclosure provides methods and diagnostic test kits for (1) the identification of bovine animals that exhibit a low probability of successful embryo implantation and maintaining pregnancy and, thus, are unsuitable candidates to act as surrogate dams for embryo transfer and (2) the selection of bovine animals that exhibit a high probability of embryo implantation and maintaining pregnancy and, thus, are suitable candidates to act as surrogate dams for embryo transfer. According to some aspects, the present disclosure provides methods utilizing a database of lipidome profiles from vaginal fluids for the selection of bovine surrogate dams and, thereby, to improve IVF efficiency by increasing the likelihood of embryo implantation and maintenance of successful pregnancies.

These and other aspects of the present disclosure can be better understood by reference to the following non-limiting definitions.

### Definitions

As used herein, the terms “in vitro fertilization” and “IVF” are used interchangeably to refer to IVF is a type of assisted reproductive technology wherein oocytes collected from a donor cow are fertilized in vitro, and the resulting embryos are implanted (single embryo implant) into the uterus of a recipient (surrogate) dam that maintains a pregnancy through to birth.

As used herein, the term “biomarker” refers to a measured characteristic that is used as an indicator of a biological state or condition. In the present disclosure, “biomarkers” include “lipid biomarkers,” and the more specific phrase “phospholipid biomarkers,” which are used diagnostically in the methodology disclosed herein for predicting IVF embryo transfer efficiency, including assessing the suitability of a bovine surrogate dam for embryo implantation as well as for maintaining a pregnancy to full term. The diagnostic methodology for quantifying lipid biomarker levels and ratios in the lipidome profile of vaginal and uterine fluids from candidate recipient bovine surrogate dams, which is disclosed herein, exhibit both good sensitivity (thereby correctly identify high fertility surrogate dams) as well as good specificity (thereby correctly identify low fertility surrogate dams).

The various lipid biomarkers that are quantified in bovine vaginal fluids include, without limitation, lipids listed in Tables 1 and 2, which provide both abbreviated, shorthand nomenclature as used throughout the present disclosure as well as the full nomenclature as used in the scientific literature and as described in Liebisch, et al., J. Lipid Research 54:1523-1530 (2013) and LipidMaps database referenced therein and present lipid ratios that are predictive of successful embryo implantation and maintenance of pregnancy.

TABLE 1
Lipids of importance (independent or in combination) in the
Lipidome from Bovine Vaginal Fluid in predicting pregnancy

| Abbreviation | Category/Class—Common Name |
|---|---|
| PC(30:1) | Phosphatidylcholines |
| PC(30:2) | |
| PC(32:2) | |
| PC(34:3) | |
| PC(34:5) | |
| PC(36:1) | |
| PC(36:3) | |
| PC(36:5) | |
| PC(36:7) | |
| PC(38:2) | |
| PC(38:5) | |
| PC(38:7) | |
| PC(40:0) | |
| PC(40:1) | |
| PC(40:10) | |
| PC(40:3) | |
| PC(40:5) | |
| PC(40:6) | |
| PC(40:7) | |
| PC(42:1) | |
| PC(42:10) | |
| PC(42:2) | |
| PC(42:3) | |
| PC(42:5) | |
| PC(42:6) | |
| PC(42:7) | |
| PC(42:8) | |
| PC(42:9) | |
| PC(44:10) | |
| PC(44:2) | |
| PC(44:3) | |
| PC(44:5) | |
| PC(44:6) | |
| Lyso PC(16:1) | Lysophosphatidylcholine |
| Lyso PC(17:0) | |
| Lyso PC(17:1) | |
| Lyso PC(17:2) | |
| Lyso PC(18:2) | |
| Lyso PC(19:2) | |
| Lyso PC(20:4) | |
| Lyso PC(22:2) | |
| Lyso PE(18:0) | Lysophosphatidylethanolamine |
| Lyso PE(22:2) | |
| PCo(32:0) | Choline plasmalogen |
| PCo(32:1) | |
| PCo(32:2) | |
| PCo(32:3) | |
| Pco(34:3) | |
| PCo(35:0) | |
| PCo(36:0) | |
| PCo(38:0) | |
| PCo(38:2) | |
| PCo(38:3) | |
| PCo(38:4) | |
| PCo(38:5) | |
| PCo(40:0) | |
| PCo(40:2) | |
| PCo(40:3) | |
| PCo(40:4) | |
| PCo(40:5) | |
| PCo(40:6) | |
| PCo(42:0) | |
| PCo(42:1) | |
| PCo(42:2) | |
| PCo(42:3) | |
| PCo(42:6) | |
| PCo(44:3) | |
| PCo(44:6) | |
| PE(18:0) | Phosphatidylethanolamines |
| PE(22:2) | |
| PE(30:1) | |
| PE(32:0) | |
| PE(32:1) | |
| PE(32:2) | |
| PE(34:2) | |
| PE(34:3) | |
| PE(34:4) | |
| PE(36:1) | |
| PE(36:3) | |
| PE(36:4) | |
| PE(36:7) | |
| PE(38:0) | |
| PE(38:6) | |
| PE(38:7) | |
| PE(38:8) | |
| PE(40:1) | |
| PE(40:4) | |
| PE(40:6) | |
| PE(40:7) | |
| PE(40:8) | |
| PE(40:9) | |
| PE(42:10) | |
| PE(42:2) | |
| PE(42:3) | |
| PE(42:4) | |
| PE(42:6) | |
| PEo(28:0) | Ethanolamine plasmalogen |
| PEo(32:1) | |
| PEo(32:5) | |
| PEo(34:0) | |
| PEo(34:3) | |
| PEo(36:0) | |
| PEo(36:5) | |
| PEo(38:0) | |
| PEo(38:1) | |
| PEo(38:4) | |
| PEo(38:6) | |
| PEo(40:0) | |
| PEo(40:1) | |
| PEo(40:2) | |
| PEo(40:4) | |
| PEo(40:6) | |
| PEp(32:1) | Phosphatidylethanolamine |
| PEp(35:5) | (PE)-plasmalogen |
| PEp(42:2) | |
| SM(d16:1/24:0) | Sphingomyelins |
| SM(d16:1/18:1) | |
| SM(d18:0/14:0) | |
| SM(d18:0/16:0) | |
| SM(d18:0/18:0) | |
| SM(d18:0/20:0) | |
| SM(d18:0/22:0) | |
| SM(d18:0/26:0) | |
| SM(d18:1/14:0) | |
| SM(d18:1/16:0) | |
| SM(d18:1/18:0) | |
| SM(d18:1/26:0) | |
| SM(d18:2/20:1) | |
| Cer(d14:1/24:0(2OH)) | Ceramides |
| Cer(d14:1/20:1(2OH)) | |
| Cer(t18:0/24:0(2OH)) | |
| Cer(d18:1/16:0) | |
| TAG(48:1)_FA 16:1 | Triacylglycerol |
| TAG(48:1)_FA 18:1 | |
| TAG(50:1)_FA 18:1 | |
| TAG(50:2)_FA 16:0 | |
| TAG(50:3)_FA 18:2 | |
| TAG(50:4)_FA 18:2 | |
| TAG(52:0)_FA 16:0 | |
| TAG(52:0)_FA 18:0 | |
| TAG(52:1) | |
| TAG(52:1)_FA 18:1 | |
| TAG(52:2)_FA 16:0 | |
| TAG(52:3)_FA 18:1 | |
| TAG(52:3)_FA 16:0 | |
| TAG(54:2)_FA 18:1 | |
| TAG(54:3)_FA 16:1 | |
| TAG(54:3)_FA 18:1 | |
| TAG(58:0)_FA 18:0 | |
| PS(20:3) | Phosphatidylserine |
| PS(22:6) | |
| PS(32:2) | |
| PS(36:3) | |
| PS(38:1) | |
| PS(40:1) | |
| PS(40:2) | |
| PS(36:0) | |
| PS(38:1) | |
| PS(38:2) | |
| PS(38:9) | |
| PI(34:2) | Phosphatidylinositol |
| PI(36:3) | |
| PI(36:8) | |
| PI(38:1) | |
| PI(38:3) | |
| PI(38:9) | |
| PI(40:2) | |
| PI(40:4) | |
| PG(34:0) | Free fatty acids and |
| | phosphatidylglycerol |
| Plo(36:1) | Plasmanylphosphatidylcholine |
| Plo(36:2) | |
| Plo(38:2) | |
| Plp(36:0) | Plasmenylphosphatidylcholine |
| Plp(36:1) | |
| Plp(38:3) | |
| Plp(38:1) | |
| Plp(38:2) | |
| 16:1 Cholesteryl ester | Cholesteryl ester |
| 18:0 Cholesteryl ester | |
| 20:0 Cholesteryl ester | |
| 20:1 Cholesteryl ester | |
| 20:3 Cholesteryl ester | |
| 22:2 Cholesteryl ester | |
| PSp(38:6) | Plasmenylserine |
| PSo(38:2) | Plasmanylserine |

Unless defined otherwise, all technical and scientific terms used above have the same meaning as commonly understood by one of ordinary skill in the art to which embodiments of the present invention pertain.

According to some aspects, the present disclosure provides a method for identifying a recipient bovine surrogate dam for embryo transfer comprising obtaining samples containing the vagina or uterus lipidome. In some embodiments, the method further comprises identifying a lipidome profile for the vaginal or uterine fluid samples from the recipient bovine surrogate dam. In some embodiments, the lipidome profiles are generated by quantifying lipid levels in fluid samples.

According to some embodiments, the lipidome profiles obtained from the recipient bovine surrogate dam are compared to a database of lipidome profiles. In some embodiments, if the lipidome profile of the recipient bovine surrogate dam is associated with a high rate of initiation of pregnancy and high maintenance of pregnancy to term, then one or more oocytes or ovum are obtained from a donor subject, fertilized by in vitro fertilization, and implanted into the recipient subject. In some embodiments, the vaginal and uterine lipidome profiles of the recipient bovine surrogate dam are indicative of high success of pregnancy after embryo transferred to the female recipient subject.

In some embodiments, the lipidome profile of the recipient bovine surrogate dam indicates a probability of embryo implantation and/or maintenance of pregnancy of greater than 50%. In some embodiments, the lipidome profile of the recipient bovine surrogate dam indicates a probability of embryo implantation and/or maintenance of pregnancy of greater than 55%. In some embodiments, the lipidome profile of the recipient bovine surrogate dam indicates a probability of embryo implantation and/or maintenance of pregnancy of greater than 60%. In some embodiments, the lipidome profile of the recipient bovine surrogate dam indicates a probability of embryo implantation and/or maintenance of pregnancy of greater than 65%. In some embodiments, the lipidome profile of the recipient bovine surrogate dam indicates a probability of embryo implantation and/or maintenance of pregnancy of greater than 70%. In some embodiments, the lipidome profile of the recipient bovine surrogate dam indicates a probability of embryo implantation and/or maintenance of pregnancy of greater than 75%. In some embodiments, the lipidome profile of the recipient bovine surrogate dam indicates a probability of embryo implantation and/or maintenance of pregnancy of greater than 80%. In some embodiments, the lipidome profile of the recipient bovine surrogate dam indicates a probability of embryo implantation and/or maintenance of pregnancy of greater than 85%. In some embodiments, the lipidome profile of the recipient bovine surrogate dam indicates a probability of embryo implantation and/or maintenance of pregnancy of greater than 90%. In some embodiments, the lipidome profile of the recipient bovine surrogate dam indicates a probability of embryo implantation and/or maintenance of pregnancy of greater than 95%. In some embodiments, the lipidome profile of the recipient bovine surrogate dam indicates a probability of embryo implantation and/or maintenance of pregnancy of greater than 99%.

In other embodiments, the lipidome profile of the recipient bovine surrogate dam indicates a probability of embryo implantation and/or maintenance of pregnancy of less than 50%. In some embodiments, the lipidome profile of the recipient bovine surrogate dam indicates a probability of embryo implantation and/or maintenance of pregnancy of less than 45%. In some embodiments, the lipidome profile of the recipient bovine surrogate dam indicates a probability of embryo implantation and/or maintenance of pregnancy of less than 40%. In some embodiments, the lipidome profile of the recipient bovine surrogate dam indicates a probability of embryo implantation and/or maintenance of pregnancy of less than 35%. In some embodiments, the lipidome profile of the recipient bovine surrogate dam indicates a probability of embryo implantation and/or maintenance of pregnancy of less than 30%. In some embodiments, the lipidome profile of the recipient bovine surrogate dam indicates a probability of embryo implantation and/or maintenance of pregnancy of less than 25%. In some embodiments, the lipidome profile of the recipient bovine surrogate dam indicates a probability of embryo implantation and/or maintenance of pregnancy of less than 20%. In some embodiments, the lipidome profile of the recipient bovine surrogate dam indicates a probability of embryo implantation and/or maintenance of pregnancy of less than 15%. In some embodiments, the lipidome profile of the recipient bovine surrogate dam indicates a probability of embryo implantation and/or maintenance of pregnancy of less than 10%. In some embodiments, the lipidome profile of the recipient bovine surrogate dam indicates a probability of embryo implantation and/or maintenance of pregnancy of less than 5%. In some embodiments, the lipidome profile of the recipient bovine surrogate dam indicates a probability of embryo implantation and/or maintenance of pregnancy of less than 1%.

In certain embodiments, the present disclosure provides methods for generating a lipidome database for identifying a recipient bovine surrogate dam to receive an embryo created via in vitro fertilization. In some aspects of these embodiments, the database is a collection of lipid information (for example, phospholipid information) that is organized so that it can be easily accessed, updated, and compared to lipids, such as a computer database. In other aspects, the lipid information in the database is obtained by collecting fluid samples from a population of recipient bovine surrogate dams, wherein the lipid samples are obtained from one or more of the vaginal and/or uterine fluid. In further aspects, the lipid information is associated with phospholipids from the fluid samples and is organized into a phospholipidome profile.

Methods for generating a lipidome database for identifying a recipient bovine surrogate dam may further comprise identifying a lipidome profile for one or more of the vaginal and/or uterine fluid samples from each subject of a population of recipient bovine surrogate dams.

Methods for generating a lipidome database for identifying a recipient bovine surrogate dam may, optionally, further comprise fertilizing one or more oocytes or ovum by in vitro fertilization and implanting the fertilized embryo(s) into one or more members of the population of recipient bovine surrogate dams and then identifying an association of the lipidome profiles with a high success rate of in vitro fertilization or a low success rate of in vitro fertilization.

In certain aspects of these embodiments that are disclosed herein, vaginal or uterine lipidome profiles of a recipient bovine surrogate dam are indicative of high success of embryo implantation following transfer via in vitro fertilization and suggest a high likelihood that a successful pregnancy will be initiated and/or maintained within the recipient bovine surrogate dam. In other aspects, vaginal or uterine lipidome profiles of a recipient bovine surrogate dam are indicative of low success of embryo implantation following transfer via in vitro fertilization and suggest a low likelihood that a successful pregnancy will be initiated and/or maintained within the recipient bovine surrogate dam. In further aspects, the lipidome profile for one or more of the vaginal or uterine phospholipid samples that is identified for recipient bovine surrogate dams may be stratified into one or more age ranges.

Within other embodiments, the present disclosure provides methods for improving the success rate of embryo implantation following in vitro fertilization. In certain aspects of these embodiments, the methods comprise obtaining vaginal or uterine fluids comprising the lipidome of a candidate recipient bovine surrogate dam. In other aspects, a lipidome profile for one or more of the vaginal or uterine fluids from the recipient bovine surrogate dam indicates that administering an intervention to the recipient bovine surrogate dam would enhance the success rate of embryo implantation and/or maintenance of pregnancy. Such interventions are effective in establishing a recipient bovine surrogate dam a lipidome profile wherein one or more lipid ratio and/or level is associated with a high success rate of in vitro fertilization, embryo implantation, and maintenance of pregnancy.

In certain aspects of the various embodiments disclosed herein, the rate of successful in vitro fertilization is measured by the incidence of embryo implantation. In other aspects, the rate of successful in vitro fertilization is measured by the initiation and/or maintenance of pregnancy. For example, in some aspects, the incidence of embryo implantation and/or initiation of pregnancy is greater than 50%. In other aspects, the incidence of embryo implantation and/or initiation of pregnancy is greater than 60%. In other aspects, the incidence of embryo implantation and/or initiation of pregnancy is greater than 70%. In other aspects, the incidence of embryo implantation and/or initiation of pregnancy is greater than 80%. In other aspects, the incidence of embryo implantation and/or initiation of pregnancy is greater than 90%. In other aspects, the incidence of embryo implantation and/or initiation of pregnancy is greater than 95%.

Interventions that may be used advantageously to increase the success rate of embryo transfer include one or more of a lipid supplementation either vaginally or orally. In certain aspects, the intervention may be administered directly to one or more of the vagina or uterus of the recipient bovine surrogate dam. In other aspects, the intervention that is administered to the recipient bovine surrogate dam is effective to provide a lipidome profile of the vaginal and/or uterine fluid wherein one or more lipid ratio and/or level is associated with high success of pregnancy after embryo transfer.

In related embodiments, the present disclosure provides diagnostic test kits for identifying recipient bovine surrogate dams for embryo transfer. In aspects of these embodiments, the diagnostic test kit comprises one or more analytical tools for determining a lipidome profile of one or more of a vaginal or uterine fluid from a recipient bovine surrogate dam. In other aspects, the one or more analytical tools for determining a lipidome profile comprise an apparatus and/or reagents for measuring lipid levels. In further aspects, the one or more analytical tools comprise a portable real-time device for quantifying lipids and the reagents for use.

Diagnostic test kits according to these embodiments may further comprise a transmitter to communicate/connect a database of one or more lipidome profiles to a device for comparing the lipidome profile of the recipient bovine surrogate dam with the database of one or more lipidome profiles to identify which recipient bovine surrogate dams have lipidome profiles indicative of a high success rate of pregnancy following embryo transfer and/or which recipient bovine surrogate dams have lipidome profiles indicative of a low success rate of pregnancy following embryo transfer.

Diagnostic test kits according to these embodiments may further comprise a database with a collection of lipid information (for example, phospholipid information) that is organized so that it can be easily accessed, updated, and compared to other lipids, such as a computer database. In some embodiments, the lipid information in the database is obtained by collecting lipid samples from a population of recipient bovine surrogate dams, wherein the lipid samples are obtained from one or more of the vaginal or uterine fluid. In aspects of these embodiments, the lipid information is associated with the ratios and levels of lipids in the samples and is organized into a lipidome profile. In other aspects, the transmitter to communicate/connect with a database is wired or wireless. In further aspects, the diagnostic test kit may further comprise instructions for use.

According to some embodiments, the present disclosure provides methods for selecting a recipient bovine surrogate dam for embryo transfer, which methods comprise (1) obtaining vaginal fluids containing a vaginal lipidome from the recipient bovine surrogate dam; (2) identifying a lipidome profile of vaginal lipids from the vaginal fluids of the recipient bovine surrogate dam; and (3) obtaining one or more embryos for embryo transfer into bovine surrogate dams in which one or more lipid ratio and/or level indicates that the recipient bovine surrogate dam exhibits a high likelihood of a successful embryo implantation and/or maintaining a pregnancy.

According to other embodiments, the present disclosure provides methods for identifying a candidate recipient bovine surrogate dam that is unsuitable for embryo transfer, which methods comprise (1) obtaining vaginal fluids containing a vaginal lipidome from the candidate recipient bovine surrogate dam; (2) identifying a lipidome profile of vaginal lipids from the vaginal fluids of the candidate recipient bovine surrogate dam; and (3) preventing the candidate recipient bovine surrogate dam from obtaining one or more embryos for embryo transfer, wherein the candidate recipient bovine surrogate dam exhibits a lipid ratio and/or level that indicates that the candidate recipient bovine surrogate dam exhibits a low likelihood of a successful embryo implantation and/or maintaining a pregnancy.

In some embodiments, the vaginal or uterine lipidome profile that is indicative of successful embryo implantation and maintenance of pregnancy in the recipient bovine surrogate dam comprises one or more elevated lipid ratios or levels, in particular one or more elevated lipid ratios or levels, including one or more increased or decreased lipid ratios or levels that are indicative of successful embryo implantation and maintenance of pregnancy in the recipient bovine surrogate dam.

Exemplified herein are methods for identifying a candidate bovine surrogate dam that exhibits an elevated lipid ratio and/or level that is predictive of successful embryo implantation following in vitro fertilization, initiation of pregnancy and, in some aspects, maintenance of a successful pregnancy up to and including full term. Also exemplified herein are methods for identifying a candidate bovine surrogate dam that exhibits an elevated lipid ratio and/or level that is predictive of unsuccessful embryo implantation following in vitro fertilization, a low likelihood of initiation of pregnancy and/or a low likelihood of maintenance of a successful pregnancy up to and including full term.

In some aspects, vaginal lipid profiles comprise a vaginal lipid ratio for a first vaginal lipid and a second vaginal lipid. In other aspects, vaginal lipid profiles comprise a vaginal lipid ratio for a first vaginal phospholipid and, in combination, a second and third lipid. In additional aspects, the vaginal lipid profiles comprise a vaginal lipid ratio for 4, 5, 6, 7, 8, 9, 10, 11, 12, 13, 14, 15, 16, 17, 18, 19 or 20 lipids. In other aspects, vaginal lipid profiles comprise a vaginal lipid ratio for more than 20 lipids. In further aspects, vaginal lipid profiles comprise a level of a single vaginal lipid.

In some embodiments of these methods, a first vaginal phospholipid is a sphingolipid, such as a sphingomyelin. In certain aspects in which the first vaginal lipid is a phospholipid, sphingomyelins include SM(d18:1/26:0) or SM(d18:1/180). In related embodiments of these methods, a second (and/or third) vaginal phospholipid is a phosphatidylethanolamine, a phosphatidylcholine, or a sphingolipid.

In some aspects, second (and/or third) vaginal phospholipids are phosphatidylethanolamines selected from a PE, a PEo, a PEp, and a Lyso PE, including PE, PEo, PEp, and Lyso PE that comprises carbon chains that are from 22 to 42 carbons in length. Specifically exemplified herein are phosphatidylethanolamines selected from PE(22:0), Lyso PE(22:2), PEo(28:0), PE(30:1), PE(32:2), PE(36:5), PEo(36:5). PE(36:5), PEo(36:5), PEp(36:5), PE(36:7), PEo(36:0), PEo(38:6), PE(38:7), PEo(38:0), PE(38:8), PEo(38:1), PE(38:0), PE(40:6), PE(40:1), PEo(40:4), PEo(40:6), PEo(40:3), PE(40:10), PE(40:8), PEo(40:1), PE(40:9), PEo(40:2), PE(42:2), PE(42:3). PE(42:4), and PE(42:5).

In other aspects, second (and/or third) vaginal phospholipids are phosphatidylcholines selected from PC, PCo, PCp, and Lyso PC, including PC, PCo, PCp, and Lyso PC that comprise carbon chains that are from 17 to 44 carbons in length. Specifically exemplified herein are phosphatidylcholines selected from Lyso PC(17:0), Lyso PC (17:1). Lyso PC (20:1), Lyso PC(22:2), PC(30:0), PC(30:2), PC(32:3), PC(38:5), PCo(32:2), PCo(32:3), PC(32:5), PC(34:5), PC(34:6), PC(36:6), PC(38:4), PC(40:5), PC(40:1), PC(40:3), PC(40:8), PCo(40:1), PC(40:0), PCp(42:6), PC(42:1), PC(42:2), PC(42:4), PCo(42:6), PC(42:10), PCo(42:0), PC(42:8), PCo(42:1), PC(44:2), PC(44:3), PC(44:4), PC(44:5), PC(44:10), PCo(44:3).

In further aspects, second (and/or third) vaginal phospholipids are sphingolipids selected from sphingomyelin, a ceramide-1-phosphate, and a sphingoid base-1-phosphate. Sphingomyelins include, for example, SM(d18:1/26:0) and SM(d18:1/18:0).

According to some embodiments, the present disclosure provides a method of generating a lipidome database for selecting a recipient bovine surrogate dam for embryo transfer comprising (1) collecting vaginal lipid samples from a population of recipient bovine surrogate dams; (2) identifying a lipidome profile for the vaginal lipid samples from each subject of the population of recipient bovine surrogate dams; and (3) identifying an association of the lipidome profile for the vaginal lipid samples with productive embryo implantation and maintenance of pregnancy. In some aspects, the lipidome profile for the vaginal lipid samples with productive embryo implantation and maintenance of pregnancy comprise an increased lipid level.

In other embodiments, the present disclosure provides a method of improving success rate of in vitro fertilization pregnancy comprising (1) obtaining vaginal lipid samples from a recipient bovine surrogate dam; (2) identifying a lipidome profile for the vaginal lipid samples; and (3) administering an intervention to the recipient bovine surrogate dam, wherein the intervention is effective to provide the recipient bovine surrogate dam with a lipidome profile associated with a high level of embryo implantation and maintenance of pregnancy. In some aspects, the intervention that is effective to provide the recipient bovine surrogate dam with a lipidome profile associated with a high level of embryo implantation and maintenance of pregnancy comprises elevating the vaginal and/or uterine levels of Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

In some embodiments, a lipid ratio that is predictive of unsuccessful embryo implantation and maintenance of pregnancy in a recipient bovine surrogate dam comprises an elevated ratio of a first phospholipid, such as a Sphingomyelin, including SM(d18:1/14:0) and SM(d18:1/6:0) a second phospholipid, such as a phosphatidylethanolamine, including PE(22:0), Lyso PE(22:2), PEo(28:0), PE(30:1), PE(32:2), PE(36:5), PEo(36:5). PE(36:5), PEo(36:5), PEp(36:5), PE(36:7), PEo(36:0), PEo(38:6), PE(38:7), PEo(38:0), PE(38:8), PEo(38:1), PE(38:0), PE(40:6), PE(40:1), PEo(40:4), PEo(40:6), PEo(40:3), PE(40:10), PE(40:8), PEo(40:1), PE(40:9), PEo(40:2), PE(42:2), PE(42:3). PE(42:4), and PE(42:5), or a phosphatidylcholine, including Lyso PC(17:0), Lyso PC (17:1). Lyso PC (20:1), Lyso PC(22:2), PC(30:2), PC(32:3), PCo(32:3), PC(32:5), PC(34:5), PC(34:6), PC(36:6), PC(40:1), PC(40:8), PCo(40:1), PC(40:0), PCp(42:6), PC(42:1), PC(42:2), PC(42:4), PCo(42:6), PC(42:10), PCo(42:0), PC(42:8), PCo(42:1), PC(44:2), PC(44:3), PC(44:4), PC(44:5), PC(44:10), PCo(44:3), or a sphingomyelin including SM(d18:1/14:0) and SM(d18:1/6:0).

According other embodiments, the present disclosure provides diagnostic test kits for selecting recipient bovine surrogate dam for embryo transfer comprising: (1) one or more analytical tools for determining a lipidome profile of a vaginal lipid sample from the recipient bovine surrogate dam; (2) a transmitter to communicate/connect a database of one or more lipidome profiles of vaginal lipid samples; (3) a device for comparing the lipidome profile of the vaginal lipid sample from the recipient bovine surrogate dam and with the database of one or more lipidome profiles to identify a recipient bovine surrogate dam has a lipidome profile including a lipid ratio and/or level that is indicative of a high or a low level of embryo implantation and maintenance of pregnancy; and optionally (4) and instructions for use.

In some aspects of these embodiments, the vaginal lipidome profile of the recipient bovine surrogate dam is associated with an elevated ratio of two or more vaginal lipids that are indicative of a low level of embryo implantation, including the ratio of a first vaginal lipid selected from Sphingomyelin, including SM(d18:1/14:0) and SM(d18:1/6:0), and a second phospholipid, such as a phosphatidylethanolamine, including PE(22:0), Lyso PE(22:2), PEo(28:0), PE(30:1), PE(32:2), PE(36:5), PEo(36:5). PE(36:5), PEo(36:5), PEp(36:5), PE(36:7), PEo(36:0), PEo(38:6), PE(38:7), PEo(38:0), PE(38:8), PEo(38:1), PE(38:0), PE(40:6), PE(40:1), PEo(40:4), PEo(40:6), PEo(40:3), PE(40:10), PE(40:8), PEo(40:1), PE(40:9), PEo(40:2), PE(42:2), PE(42:3). PE(42:4), and PE(42:5), or a phosphatidylcholine, including Lyso PC(17:0), Lyso PC (17:1). Lyso PC (20:1), Lyso PC(22:2), PC(30:2), PC(32:3), PCo(32:3), PC(32:5), PC(34:5), PC(34:6), PC(36:6), PC(40:1), PC(40:8), PCo(40:1), PC(40:0), PCp(42:6), PC(42:1), PC(42:2), PC(42:4), PCo(42:6), PC(42:10), PCo(42:0), PC(42:8), PCo(42:1), PC(44:2), PC(44:3), PC(44:4), PC(44:5), PC(44:10), PCo(44:3), or a sphingomyelin including SM(d18:1/14:0) and SM(d18:1/6:0).

In other aspects of these embodiments, the vaginal lipidome profile of the recipient bovine surrogate dam is associated with an increased or decreased level of one or more vaginal lipids that are indicative of a high level of embryo implantation, including, for example, an increase in Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

### Embodiments

The following numbered embodiments form part of the present disclosure.

1. A method for predicting in a candidate bovine surrogate dam an ability to establish a successful embryo implantation and pregnancy following embryo transfer, the method comprising: (a) obtaining vaginal fluids from a bovine surrogate dam, wherein the vaginal fluids comprises a vaginal lipidome; and (b) performing lipidome analysis on the vaginal fluids to obtain a vaginal lipid profile comprising vaginal lipid ratios, wherein an increase or decreased vaginal lipid ratio and/or individual lipid concentration in the candidate bovine surrogate dam animal is predictive of a candidate bovine surrogate dam possessing an environment that is suitable for achieving a successful embryo implantation and pregnancy or that is unsuitable for achieving a successful embryo implantation and pregnancy.

2. The method of embodiment 1 wherein the vaginal lipid profile comprises a vaginal lipid profile comprising a vaginal lipid ratio for a first vaginal lipid and a second vaginal lipid that is predictive of a candidate bovine surrogate dam possessing an environment that is suitable or unsuitable for achieving a successful embryo implantation and pregnancy.

3. The method of embodiment 2 wherein the first vaginal lipid is a sphingolipid.

4. The method of embodiment 3 wherein the sphingolipid is a sphingomyelin.

5. The method of embodiment 4 wherein the sphingomyelin is selected from the group consisting of SM(d18:1/26:0) and SM(d18:1/18:0).

6. The method of embodiment 2 wherein the second vaginal lipid is selected from the group consisting of a Hosphatidylcholine, Lysophosphatidylcholine, Lysophosphatidylethanolamine, Choline plasmalogen, Phosphatidylethanolamines, Ethanolamine plasmalogen, Phosphatidylethanolamine (PE)-plasmalogen, Sphingomyelin, Ceramides, Triacylglycerol, Phosphatidylserine, Phosphatidylinositol, Free fatty acids and phosphatidylglycerol, Plasmanylphosphatidylcholine, Plasmenylphosphatidylcholine, Cholesteryl ester, Plasmenylserine, and a Plasmanylserine.

7. The method of embodiment 6 wherein the second vaginal lipid is a phosphatidylcholine.

8. The method of embodiment 7 wherein the phosphatidylethanolamine is selected from the group consisting of SM(d18:1/26:0)/SM(d18:1/18:0), SM(d18:0/24:0)/SM(d18:1/18:0), PCo (32:2)/PC(38:5), SM(d16:1/18:1)/SM(d18:1/18:0), PC(38:5)/PC(30:0), PC(40:5)/SM(d18:1/18.0), PCo(32:2)/PC(40.5), SM(d18:1/18:0)/PC(38:2), PC(38:5)/SM(d18:1/18:0), PC(34:5)/PE(34.4), PCo(32.2)/PC(38.4), PCo(32:2)/PC(38.4), SM(d18:1/18:0)/PC(38.5), and PC(40.3)/SM(d18:1/18:0).

9. The method of embodiment 1 wherein the vaginal lipid profile comprises comprises multiple vaginal lipid ratios for at least two vaginal lipid ratios.

10. The method of embodiment 1 wherein the vaginal lipid profile comprises a vaginal lipid profile comprising a vaginal lipid level that is predictive of a candidate bovine surrogate dam possessing an environment that is suitable for achieving a successful embryo implantation and pregnancy.

11. The method of embodiment 10 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

12. The method of embodiment 1 wherein the vaginal lipid profile comprises a vaginal lipid profile comprising a vaginal lipid level that is predictive of a candidate bovine surrogate dam possessing an environment that is not suitable for achieving a successful embryo implantation and pregnancy.

13. The method of embodiment 1 wherein the lipidome analysis comprises mass spectroscopy.

14. The method of embodiment 13 wherein vaginal fluid sample data is delivered to a mass spectrometer from a handheld device.

15. The method of embodiment 13 wherein the mass spectrometry is conducted through a portable device.

16. A method for generating a lipidome database for identifying a bovine surrogate dam for embryo transfer, the method comprising: (a) collecting vaginal fluids individually from a population of bovine animals, wherein the vaginal fluids comprise a lipidome of each of the bovine animals; (b) performing lipidome analysis on the vaginal fluids to obtain a vaginal lipid profile, wherein an increased or decreased concentration in the vaginal lipid profile is predictive of a bovine surrogate dam possessing a suitable or unsuitable environment for achieving a successful embryo implantation and pregnancy; (c) delivering an embryo to at least one bovine animal; and (d) identifying an association of vaginal lipids in the vaginal lipidome profile with an increased probability of a successful embryo implantation and pregnancy following embryo delivery.

17. The method of embodiment 16 wherein the lipidome analysis comprises mass spectroscopy.

18. The method of embodiment 17 wherein vaginal fluid sample data is delivered to a mass spectrometer from a handheld device.

19. The method of embodiment 17 wherein the mass spectrometry is conducted through a portable device.

20. The method of embodiment 16 wherein the vaginal lipid profile comprises a vaginal phospholipid profile comprising a vaginal lipid level that is predictive of a candidate bovine surrogate dam possessing an environment that is suitable for achieving a successful embryo implantation and pregnancy.

21. The method of embodiment 16 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

22. A method for improving success rate of embryo transfer after in vitro fertilization in one or more bovine animals, the method comprising: (a) collecting vaginal fluid from each of the bovine animals, wherein the vaginal fluid comprises a lipidome of each the bovine animal; (b) performing lipidome analysis on the vaginal fluids to obtain a vaginal lipid profile, wherein an increase or decrease in the vaginal lipid profile is predictive of a bovine surrogate dam possessing a suitable environment or an unsuitable environment for achieving a successful embryo implantation and pregnancy following in vitro fertilization; and (c) administering an intervention to one or more of the bovine surrogate dams, wherein the intervention is effective to provide a recipient bovine surrogate dam with a lipidome profile associated with a high success rate of embryo transfer.

23. The method of embodiment 22 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

24. The method of embodiment 22 wherein the lipidome analysis comprises mass spectroscopy.

25. The method of embodiment 24 wherein vaginal fluid sample data is delivered to a mass spectrometer from a handheld device.

26. The method of embodiment 24 wherein the mass spectrometry is conducted through a portable device.

27. The method of embodiment 22, wherein the intervention that is administered to the bovine surrogate dam is effective to provide a lipidome profile of the vaginal fluid exhibiting one or more lipid ratios and/or levels that are associated with high success of pregnancy after embryo transfer, including increased Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

28. A diagnostic test kit for identifying a bovine surrogate dam having a high probability or a low probability of establishing a successful embryo implantation and pregnancy following embryo transfer via in vitro fertilization, the diagnostic test comprising: (a) one or more analytical tools for determining a vaginal lipid profile comprising vaginal lipid ratios and/or levels from the bovine animal; (b) a transmitter to communicate with a database of one or more vaginal lipid levels and/or ratios of the vaginal fluid; and (c) a device for comparing the vaginal lipid ratios and/or levels of the vaginal fluid from the surrogate dam with a database of vaginal lipid ratios identify a surrogate dam having a vaginal lipid ratio that is predictive of a high pregnancy success rate or a low pregnancy success rate after receiving an in vitro fertilization derived embryo.

29. The diagnostic test kit of embodiment 28 wherein the vaginal lipid profile comprises a vaginal lipid ratio for a first vaginal lipid and a second vaginal lipid.

30. The diagnostic test of embodiment 28 wherein the lipidome analysis comprises mass spectroscopy.

31. The diagnostic test kit of embodiment 30 wherein vaginal fluid sample data is delivered to a mass spectrometer from a handheld device.

32. The diagnostic test kit of embodiment 30 wherein the mass spectrometry is conducted through a portable device.

33. The method of embodiment 1, 16 or 22, wherein the bovine surrogate dam is a beef female.

34. The method of embodiment 1, 16 or 22, wherein the bovine surrogate dam is a dairy female.

35. The diagnostic test kit of embodiment 28, wherein the bovine surrogate dam is a beef female.

36. The diagnostic test kit of embodiment 28, wherein the bovine surrogate dam is a dairy female.

While various embodiments have been disclosed herein, other embodiments will be apparent to those skilled in the art. The various embodiments disclosed herein are for purposes of illustration and are not intended to be limiting, with the true scope and spirit being indicated by the claims. Moreover, the embodiments described in this disclosure can be combined in various ways. Any aspect or feature that is described for one embodiment can be incorporated into any other embodiment mentioned in this disclosure. While various aspects of presently disclosed embodiments are described herein, it will be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation. The disclosures of all patent and scientific literature cited herein are expressly incorporated by reference in their entirety.

### EXAMPLES

The present disclosure is further described with reference to the following examples, which are provided to illustrate certain embodiments and are not intended to limit the scope of the present disclosure or the subject matter claimed.

### Example 1

### Analysis of Lipid Levels and Ratios that are Associated with a High or Low Rate of Embryo Implantation and Maintenance of Pregnancy in Vaginal Fluid of Candidate Cows

This Example demonstrates that certain lipid levels and ratios in the vaginal fluid of a candidate cow is predictive of a high or low rate of embryo implantation following in vitro fertilization and high or low probability, respectively, that a pregnancy will be maintained to full term. Specific vaginal lipid profiles were identified that are reflective or predictive of the ability of a surrogate dam to establish a successful pregnancy following embryo transfer.

A diagnostic test using mass spectrometry allows field or onsite collection of vaginal fluids and subsequent laboratory analysis to establish lipid profiles and ratios that convey the ability or inability of a recipient bovine animal to become pregnant via an embryo transfer and thus are desirable candidate animals for serving as surrogate dams for in vitro fertilization derived embryos.

Specific vaginal lipid profiles have been identified that reflect/predict the ability or inability of a surrogate dam to recognize and maintain an embryo pregnancy. The specific lipid profiles are able to categorize a surrogate dam as being likely or not likely to become pregnant with approximately an 80% accuracy. Currently the selection method available to identify if a surrogate dam is a candidate for embryo transfer is the presence of a Corpus Luteum at time of transfer. The ability to identify the lipid profile of a surrogate dam at or prior to embryo transfer can increase the selection accuracy of candidate surrogate dams resulting in a higher overall pregnancy rate.

A diagnostic test using mass spectrometry instrumentation and techniques allows field or onsite collection of vaginal fluids and subsequent lipidome analysis to establish lipid profiles and ratios that convey a symbiotic environment between the surrogate dam and embryo which can lead to maternal recognition of pregnancy and intrauterine embryo implantation.

The use of the vaginal lipid profiles to identify superior and inferior surrogate dams with a high accuracy (80%) will increase pregnancy rates and will increase the likelihood of high genetic merit embryos implanting and eventually calving.

Area under the curve (AUC) analysis is used to assess the degree to which a lipid ratio stratifies given a phenotype (pregnancy status in this incidence). Ratios with an AUC of >0.7 would be considered accurate and those ratios were selected for to evaluate their classification accuracy. Box plots display the significant difference (p<0.05) in the concentration (and normalized concentration) of the lipids Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and TAG(52:3)_FA 16:0; indicating a significantly higher concentration in the pregnant females, where “0” on the X axis indicates lack of pregnancy, and “1” indicates successful pregnancy. On the Y axis of the boxplots, the concentration is milligrams of lipid per deciliter, and the axis on the right indicates the normalized concentration value (See, Table 2).

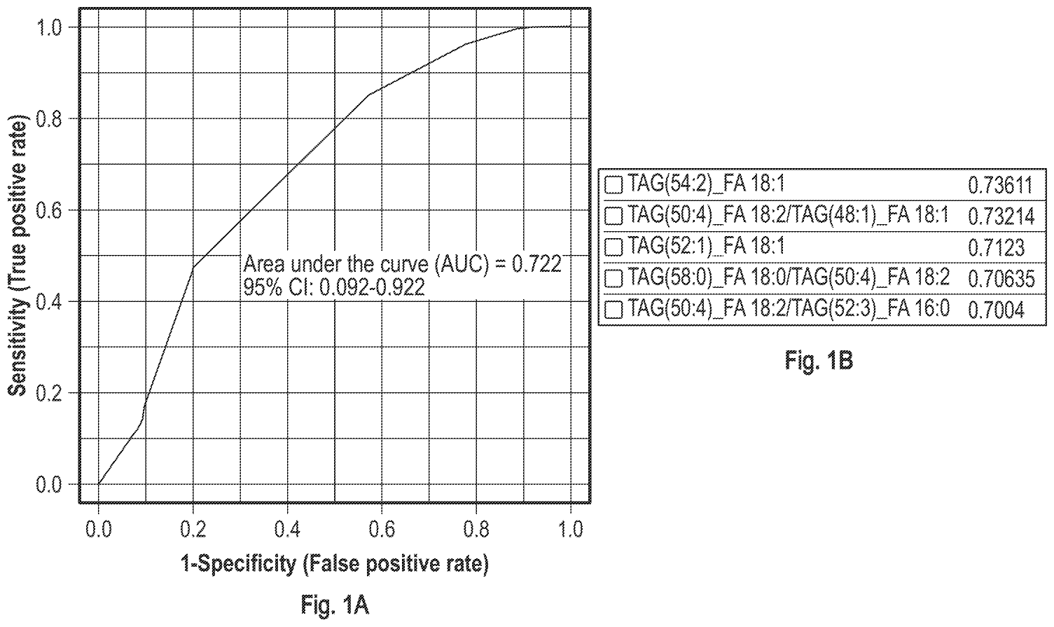

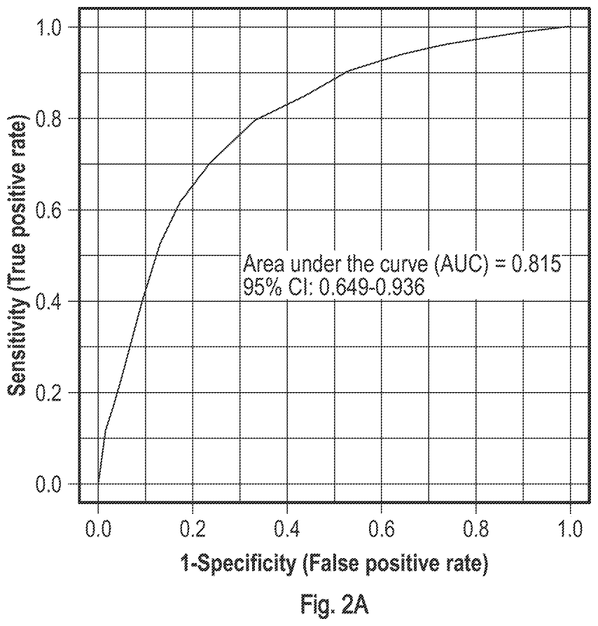

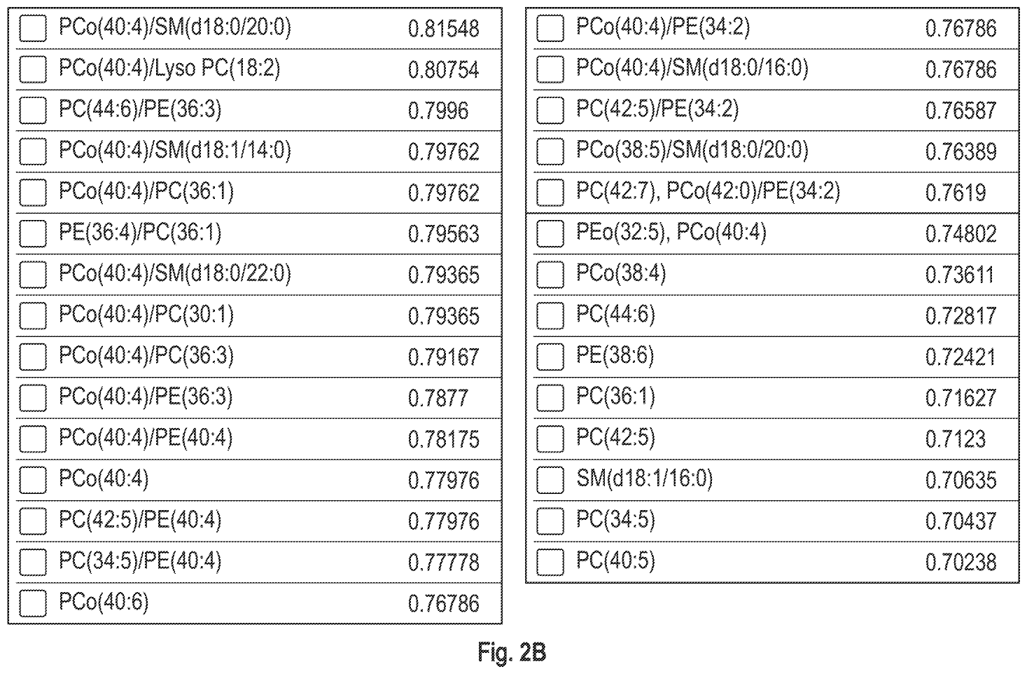

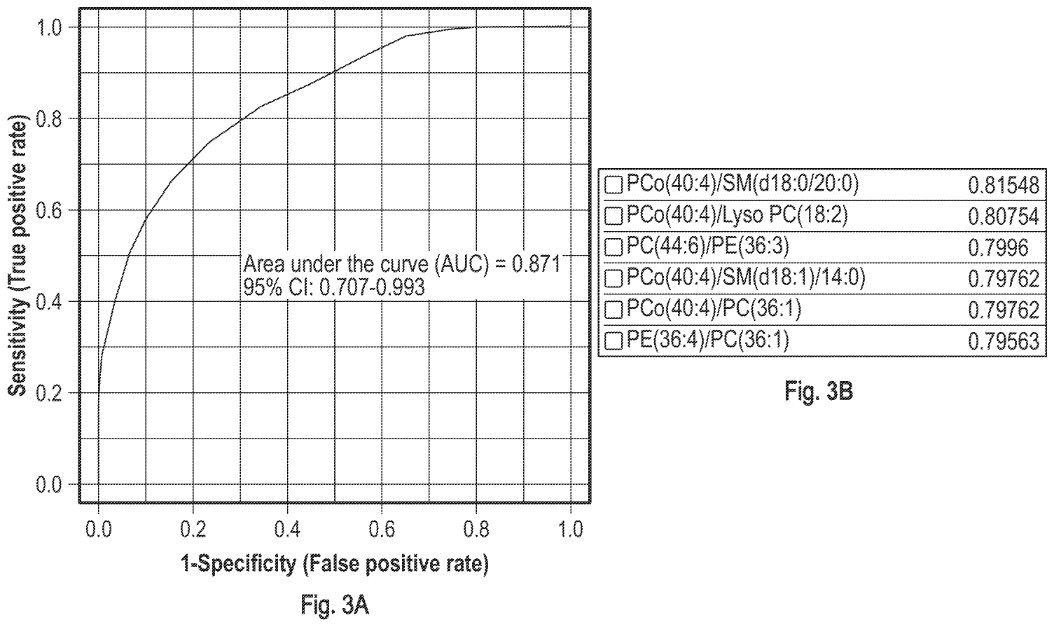

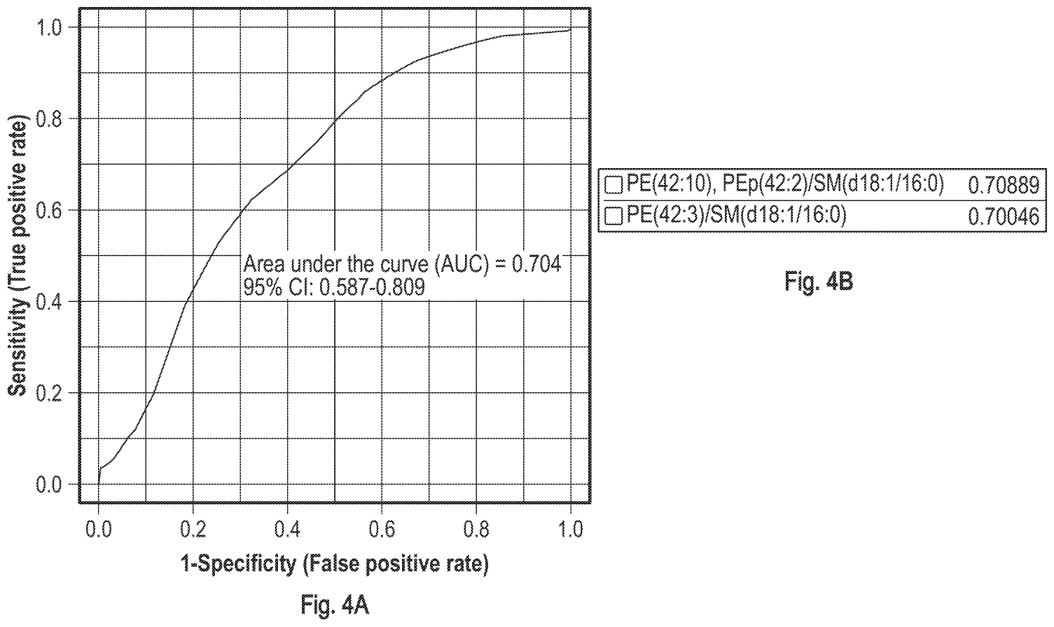

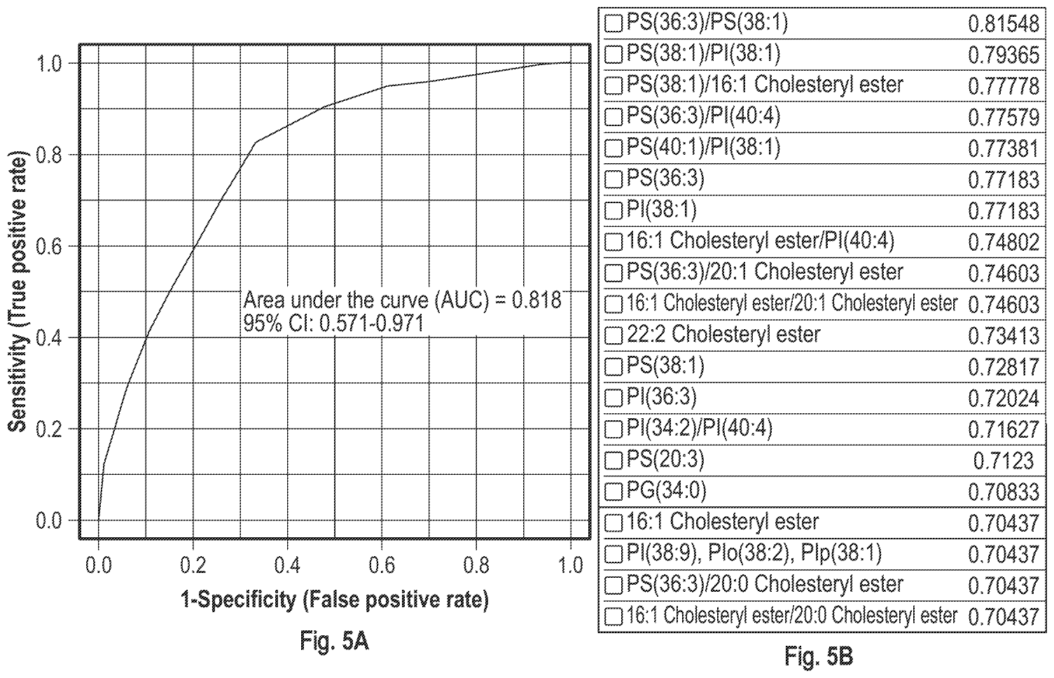

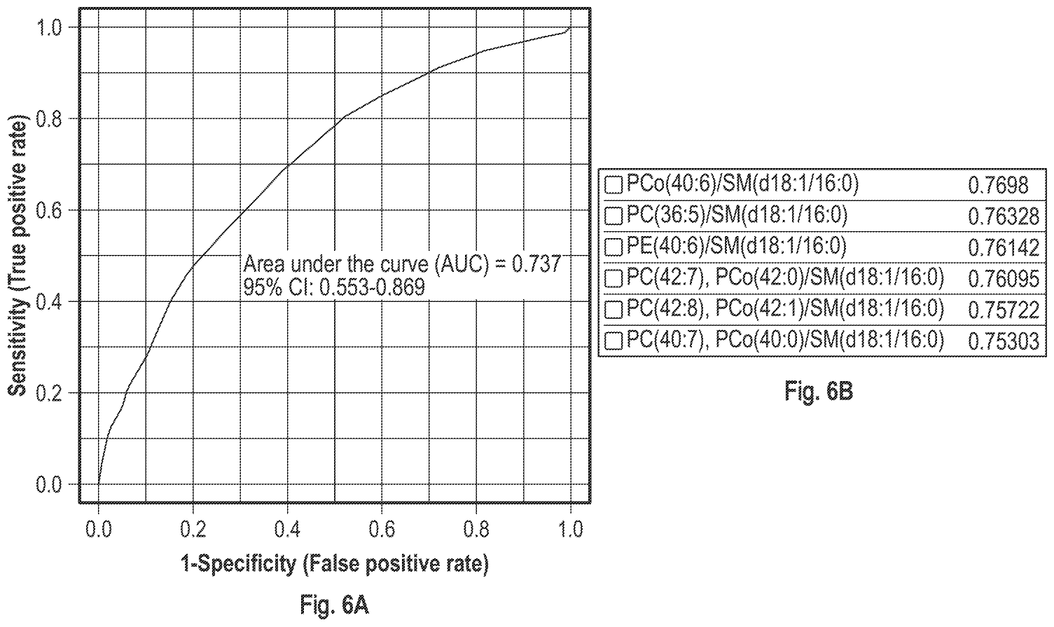

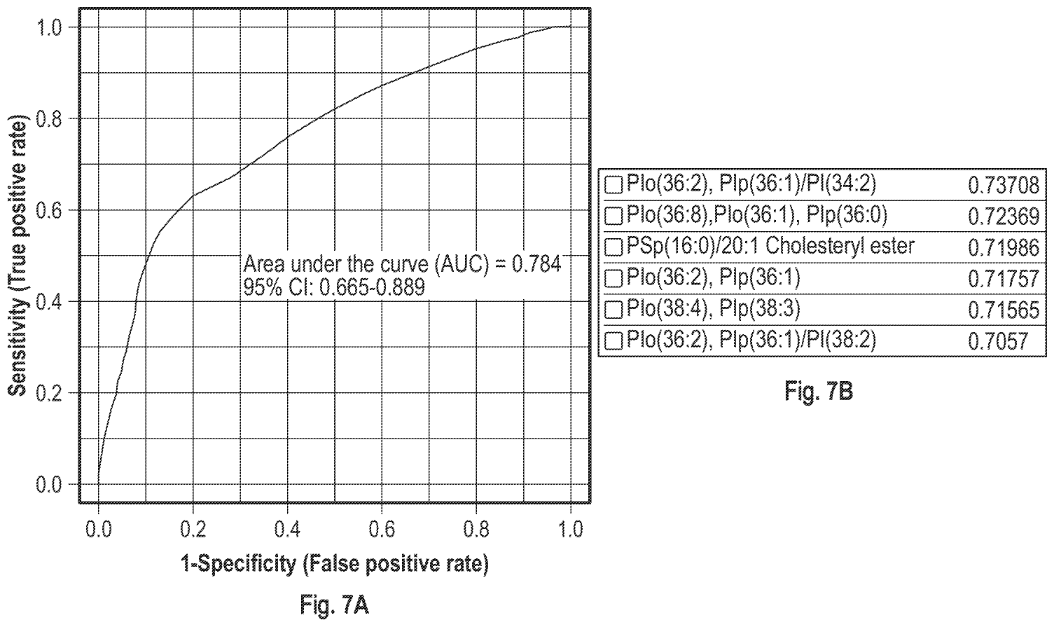

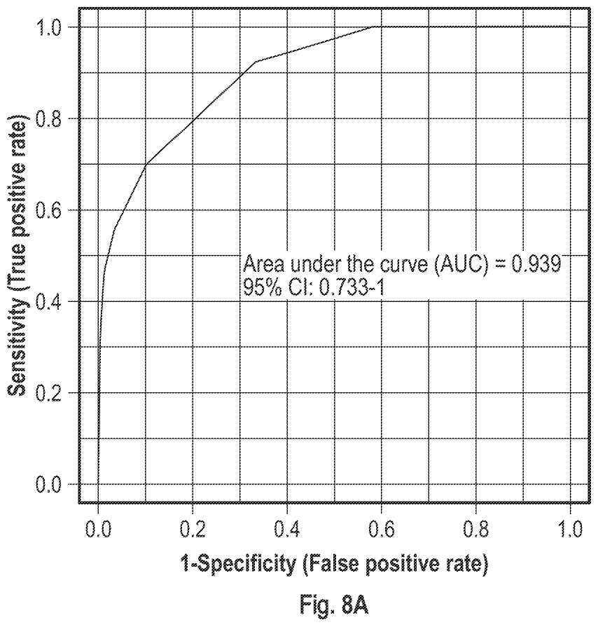

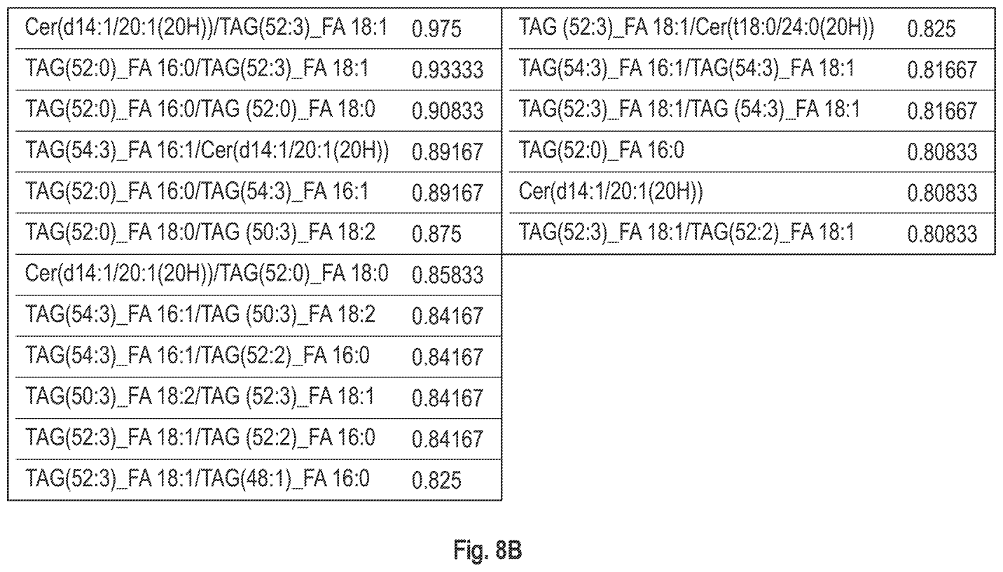

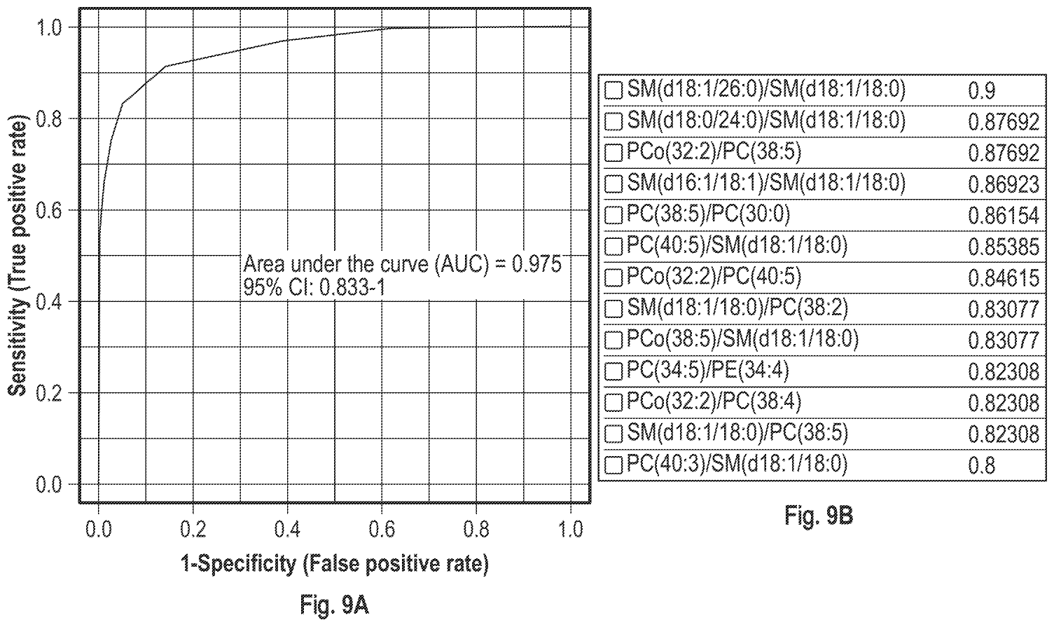

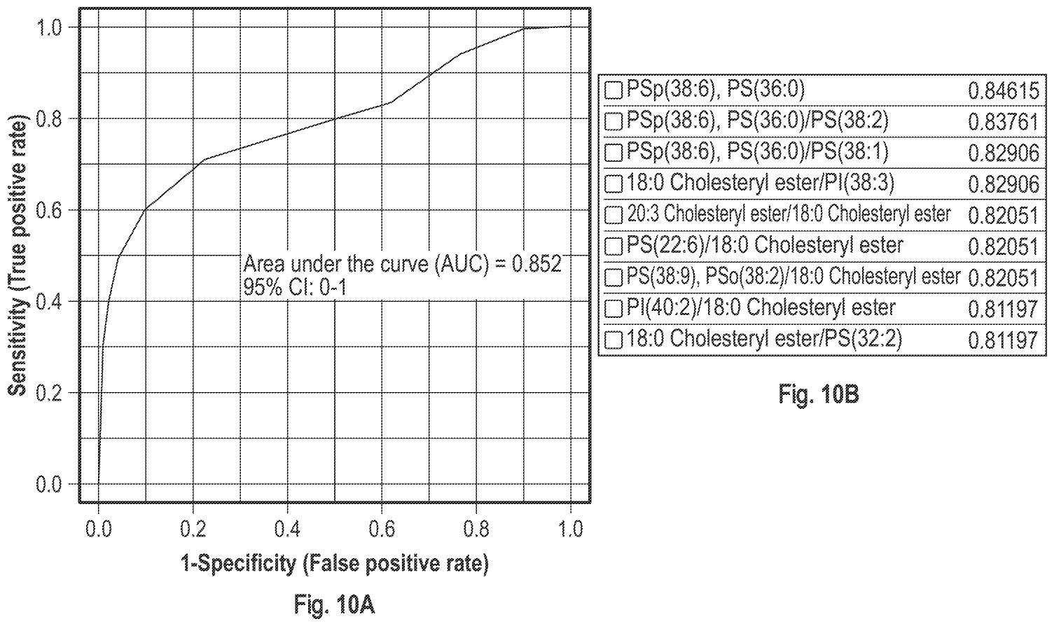

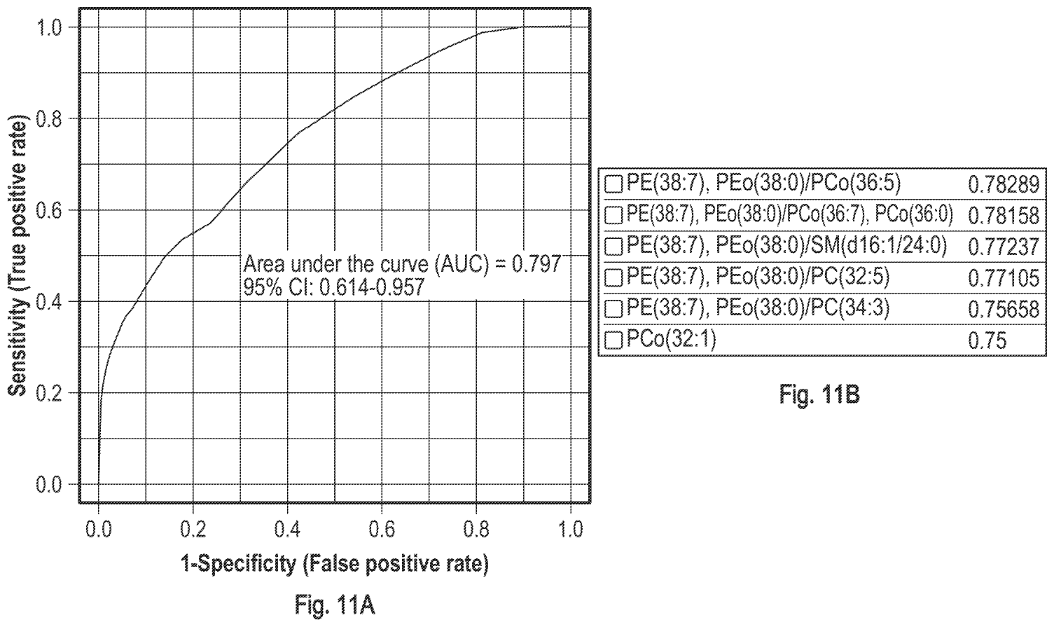

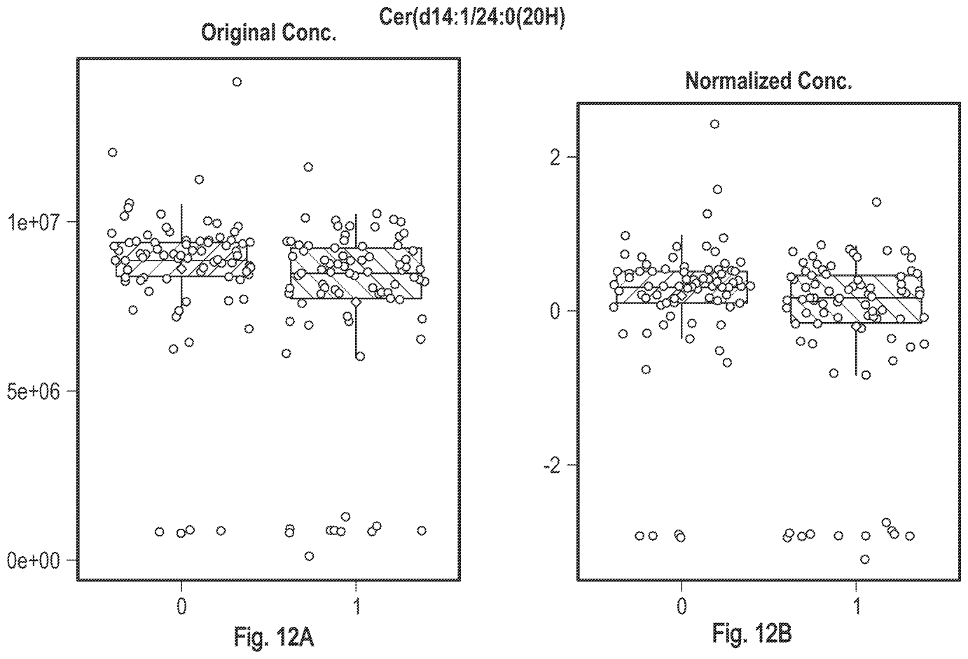

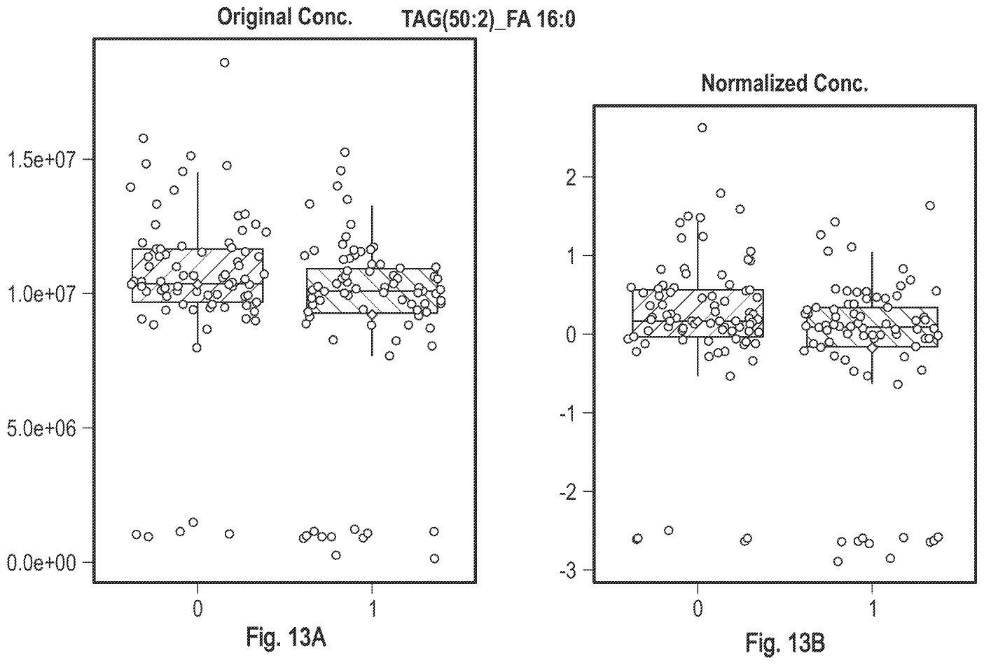

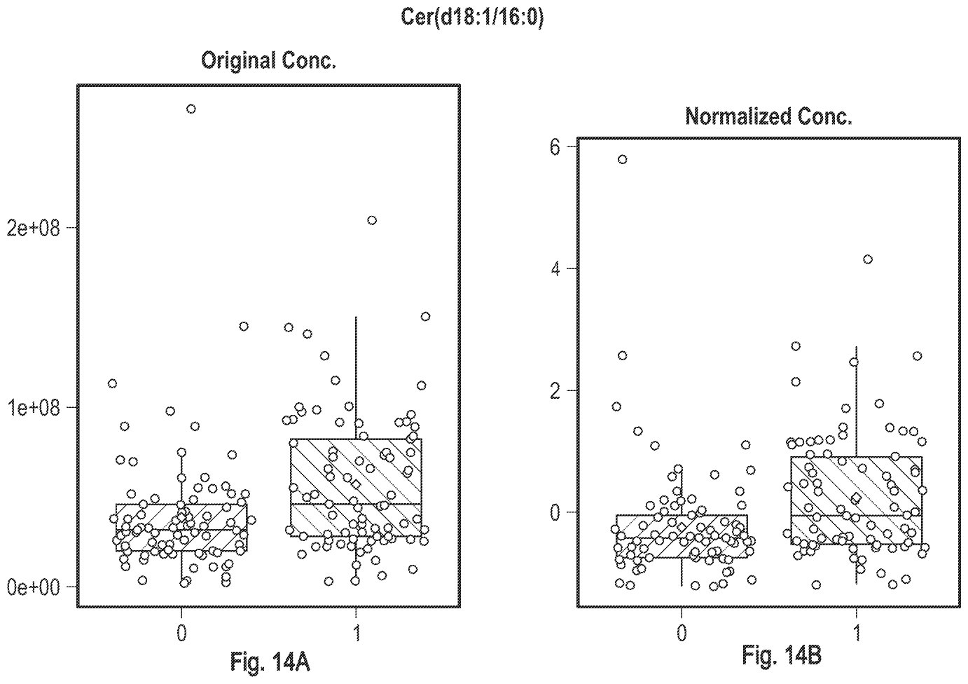

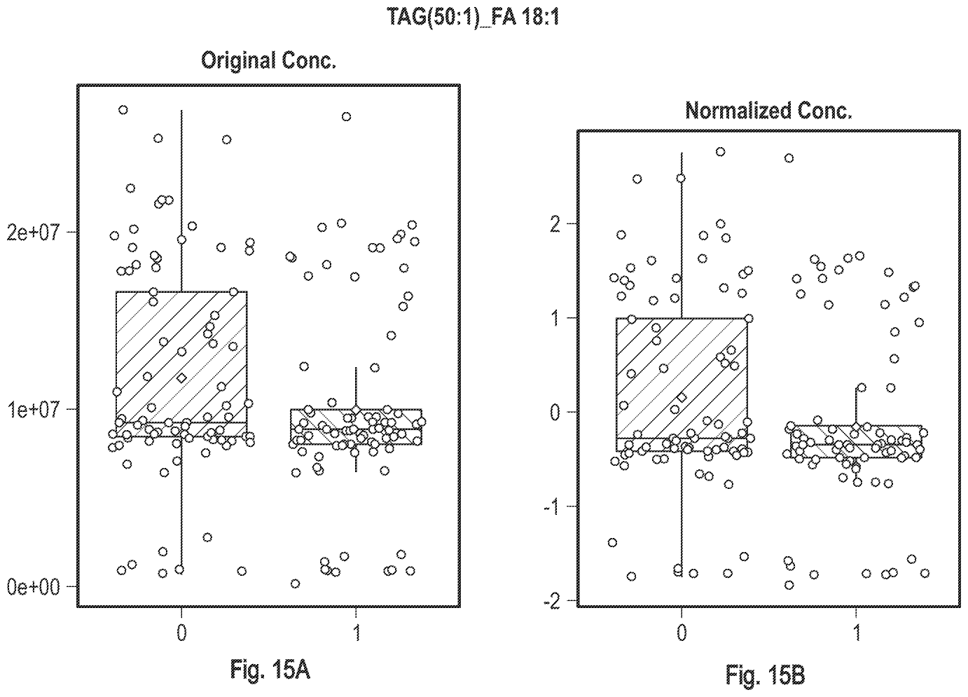

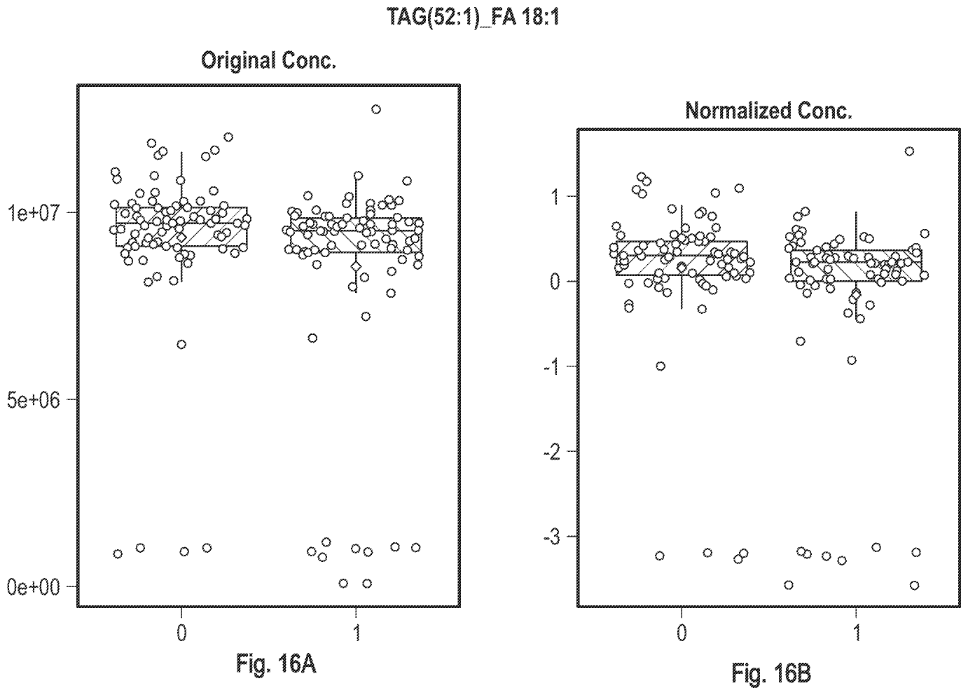

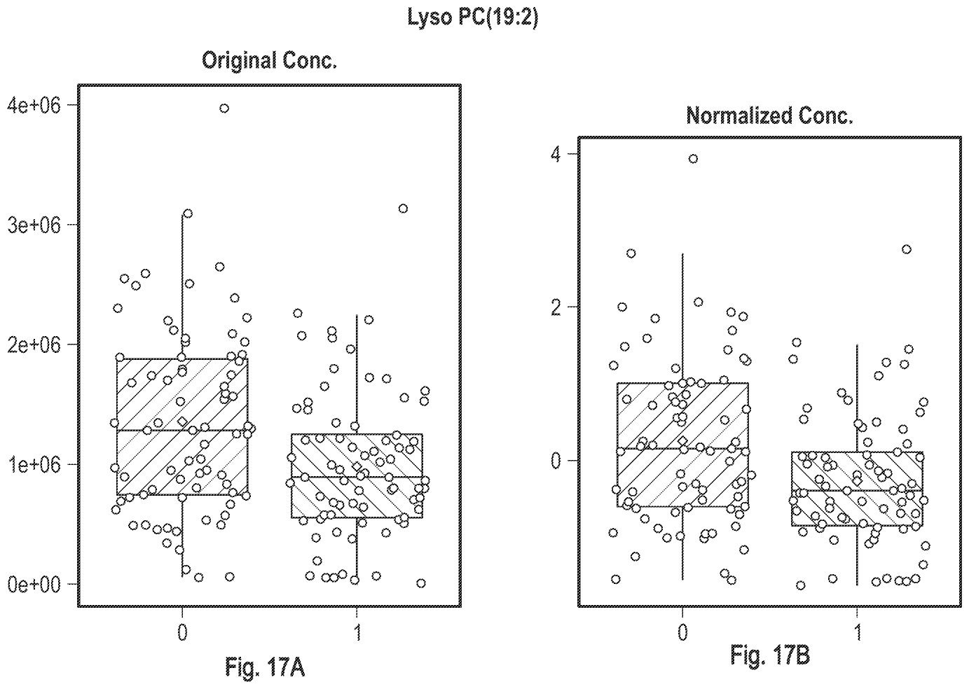

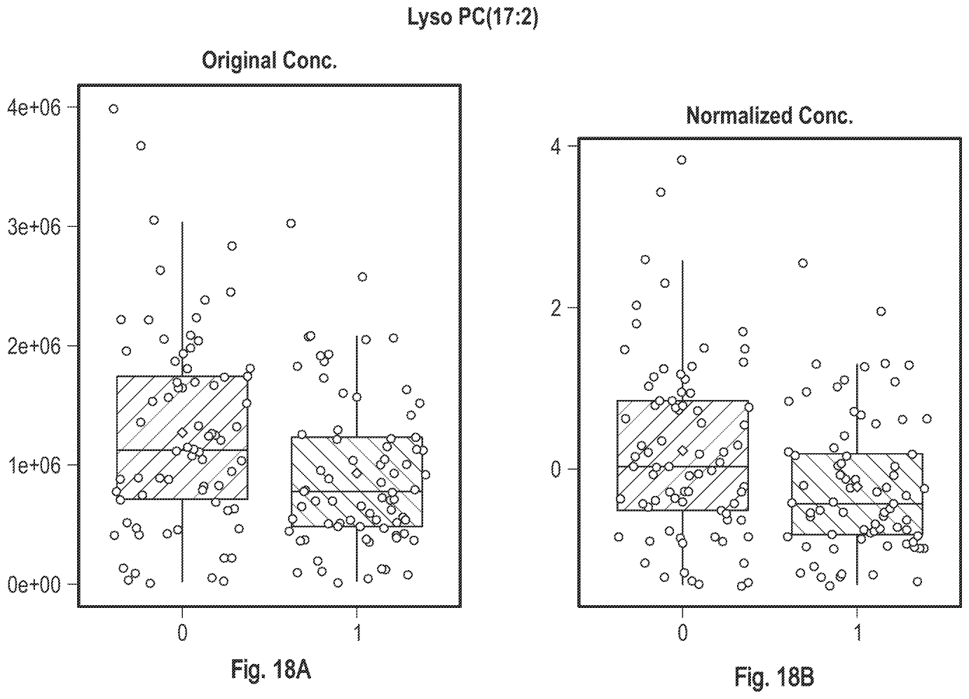

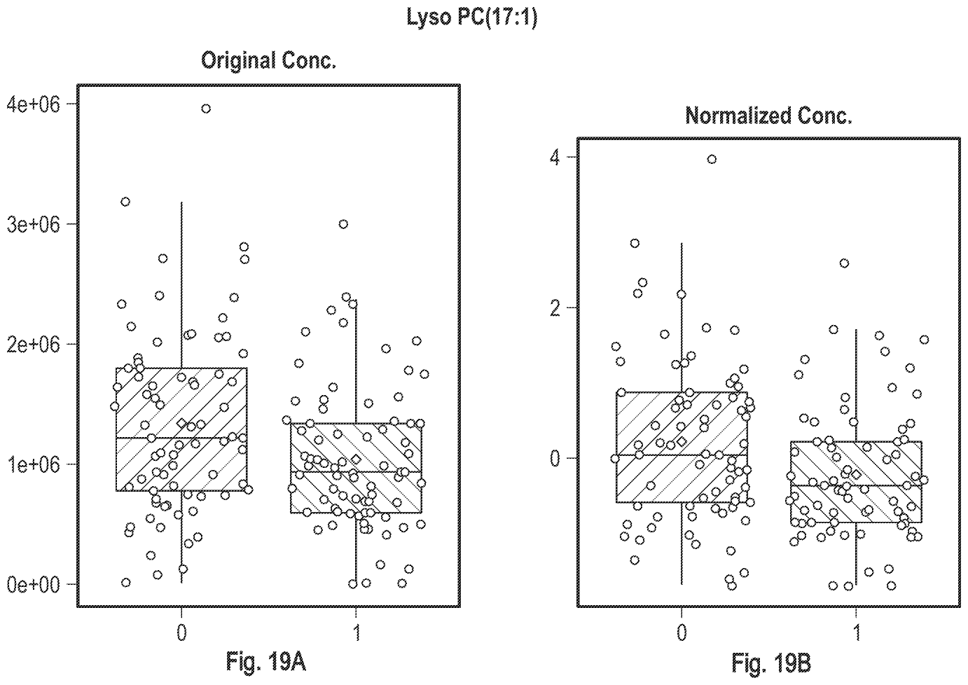

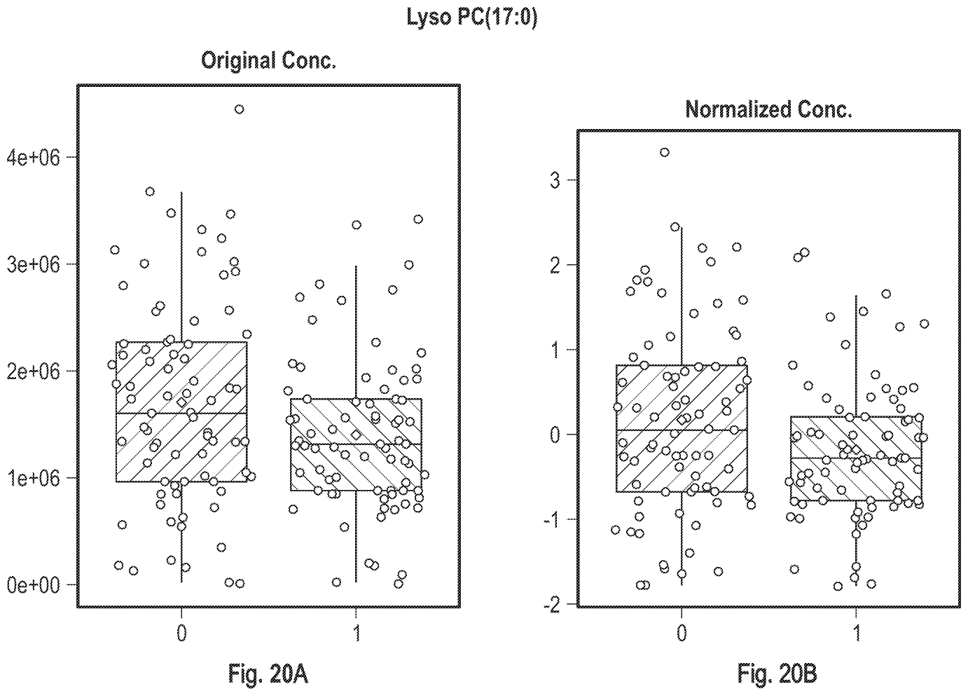

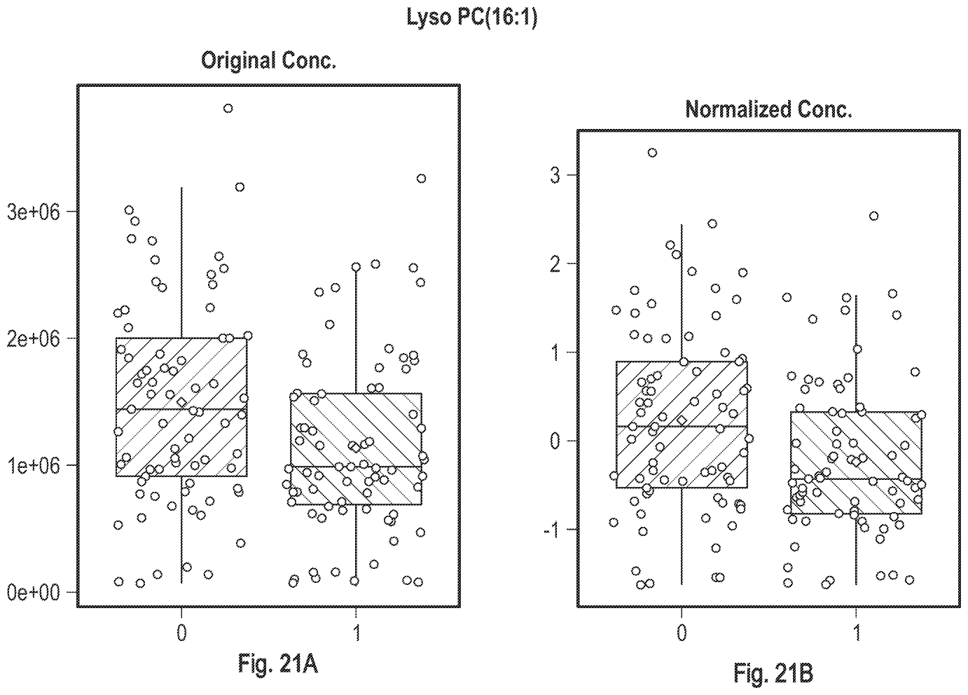

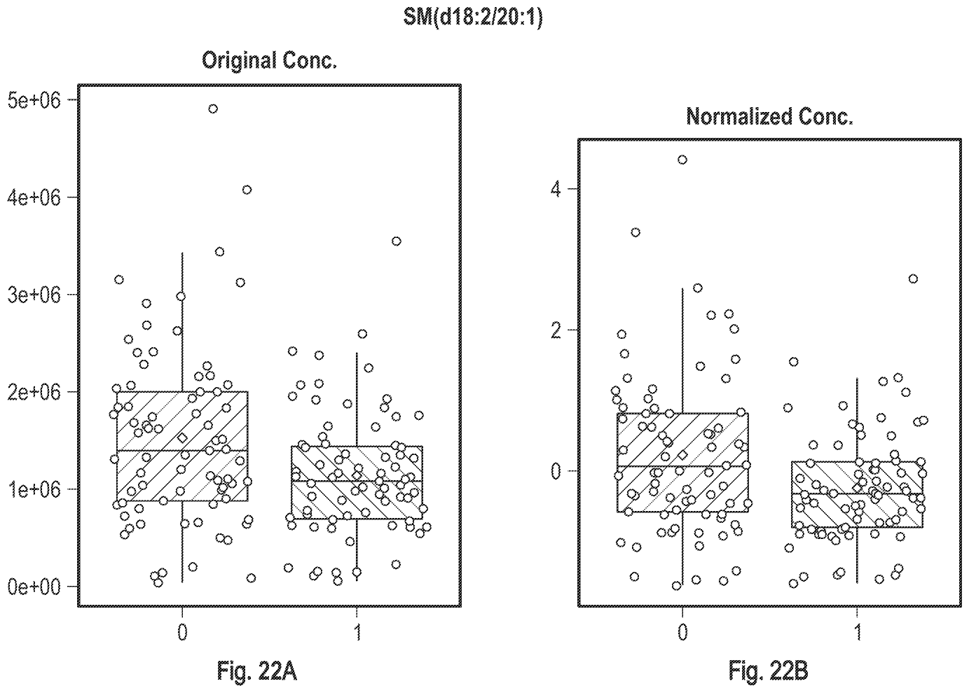

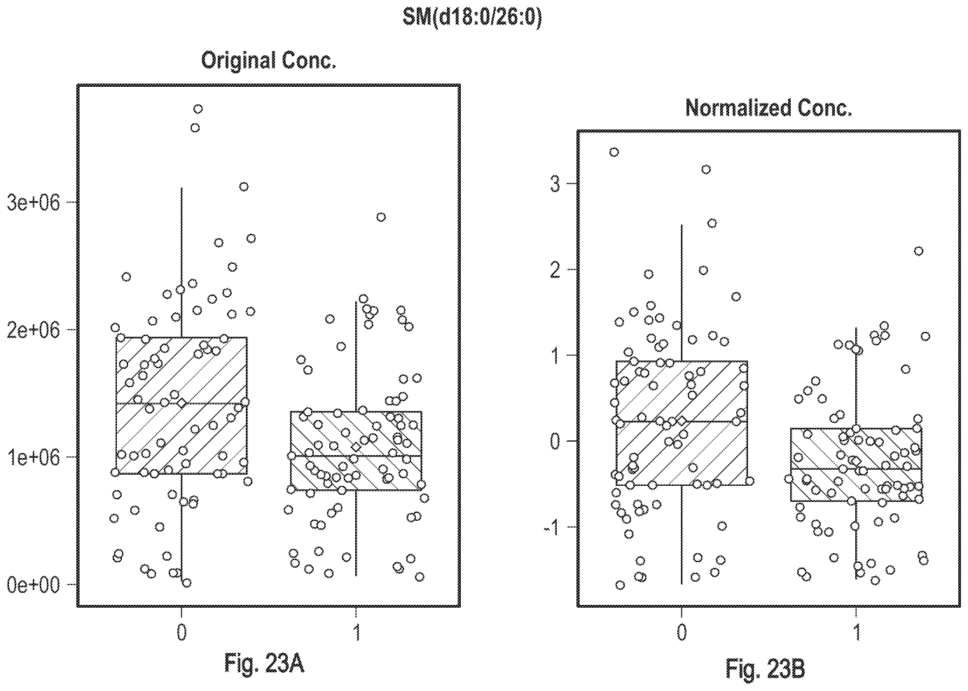

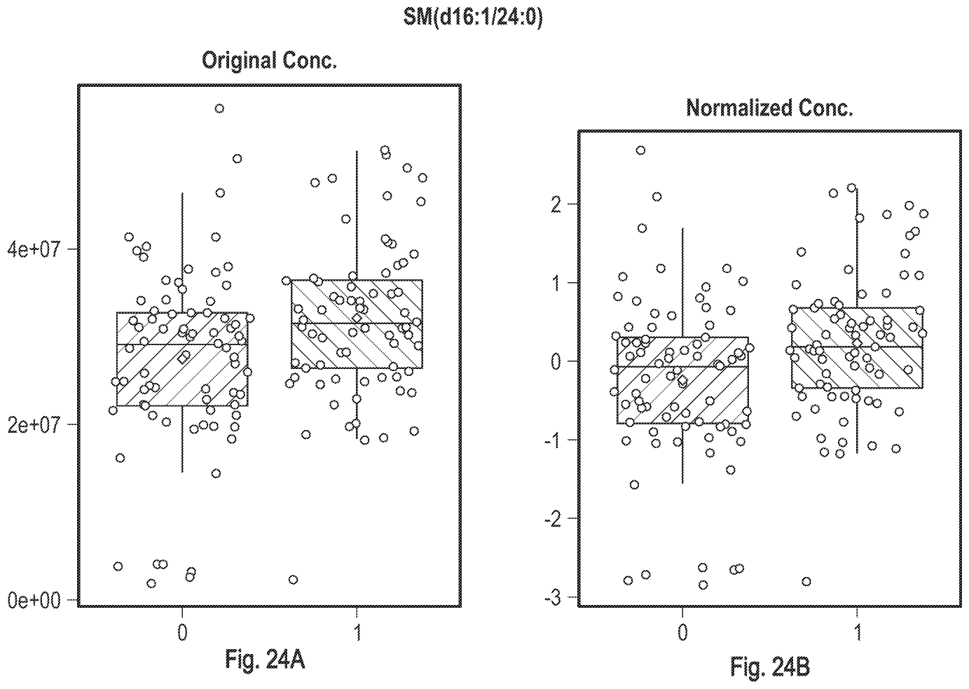

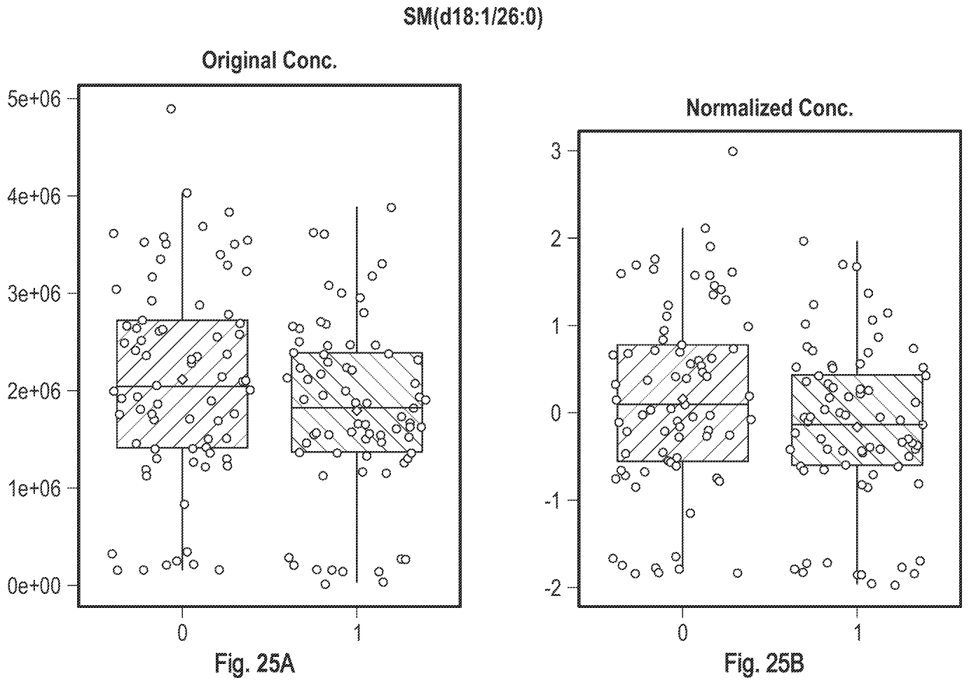

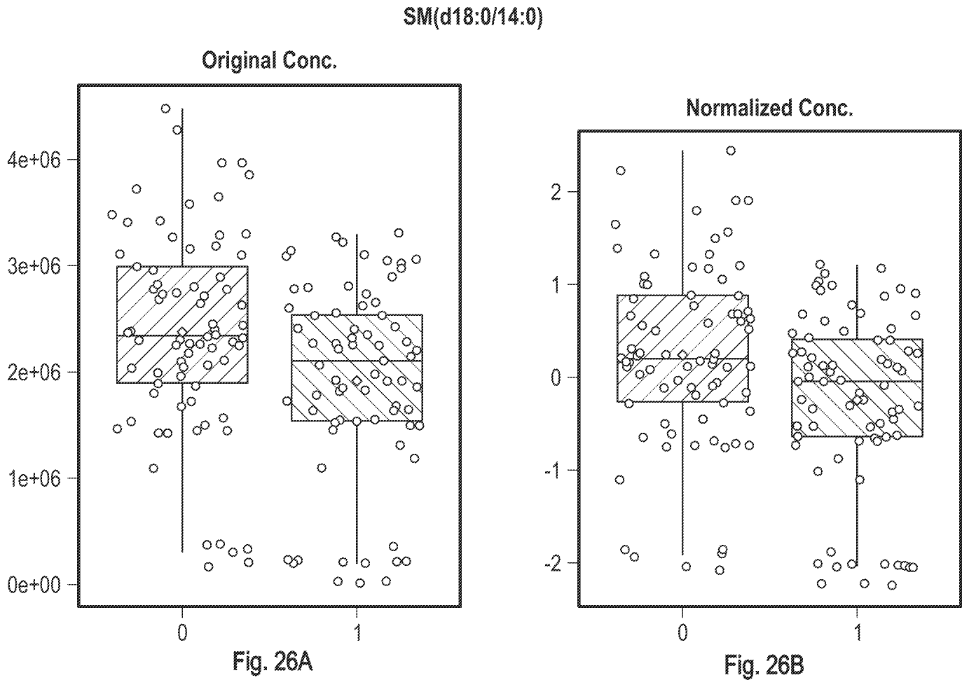

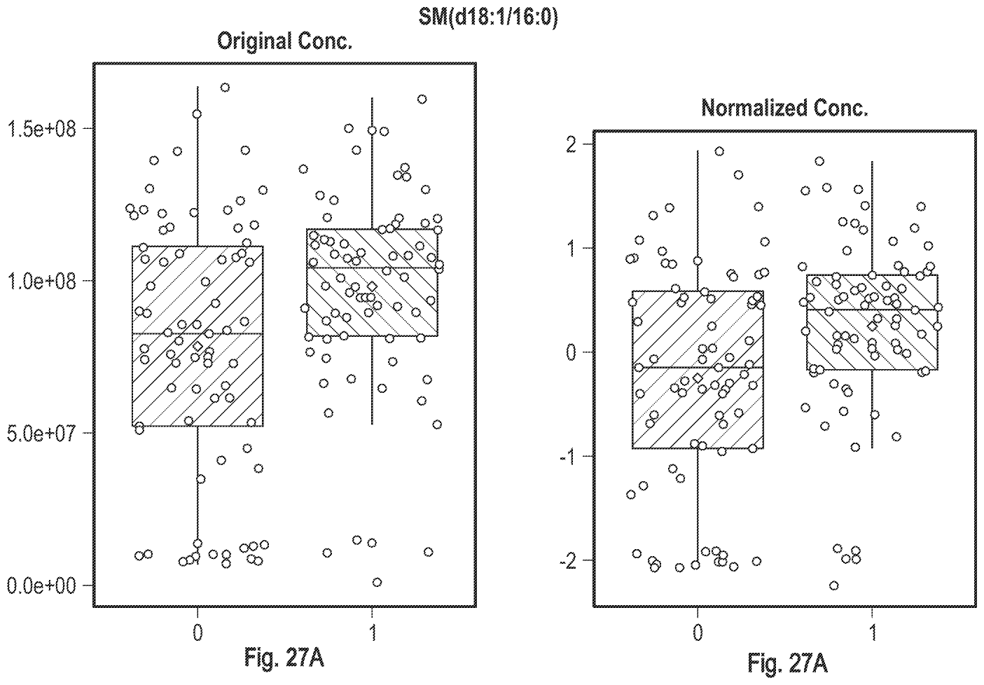

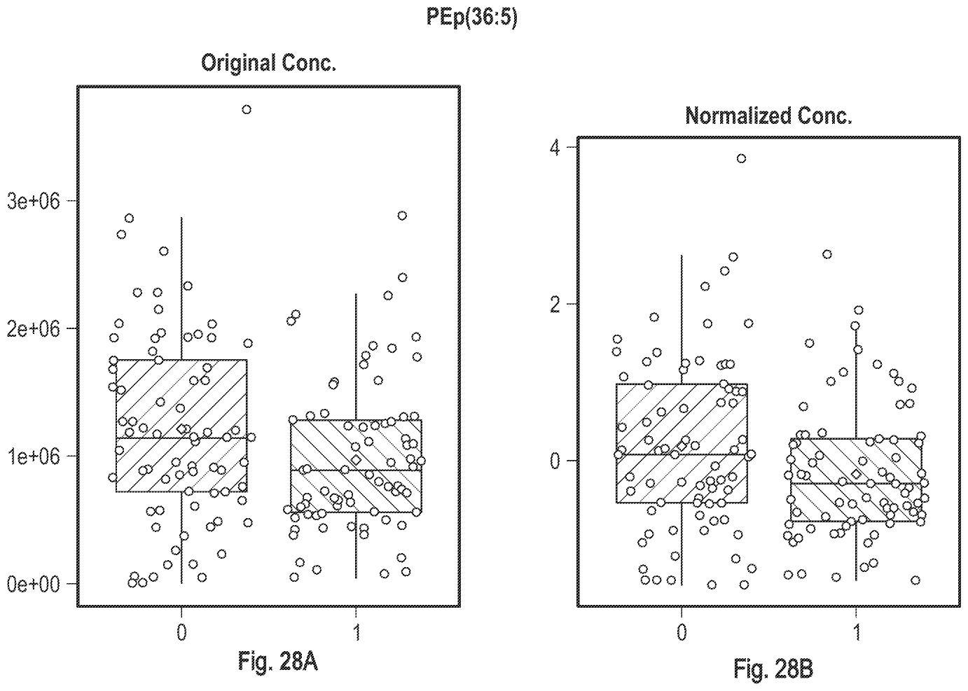

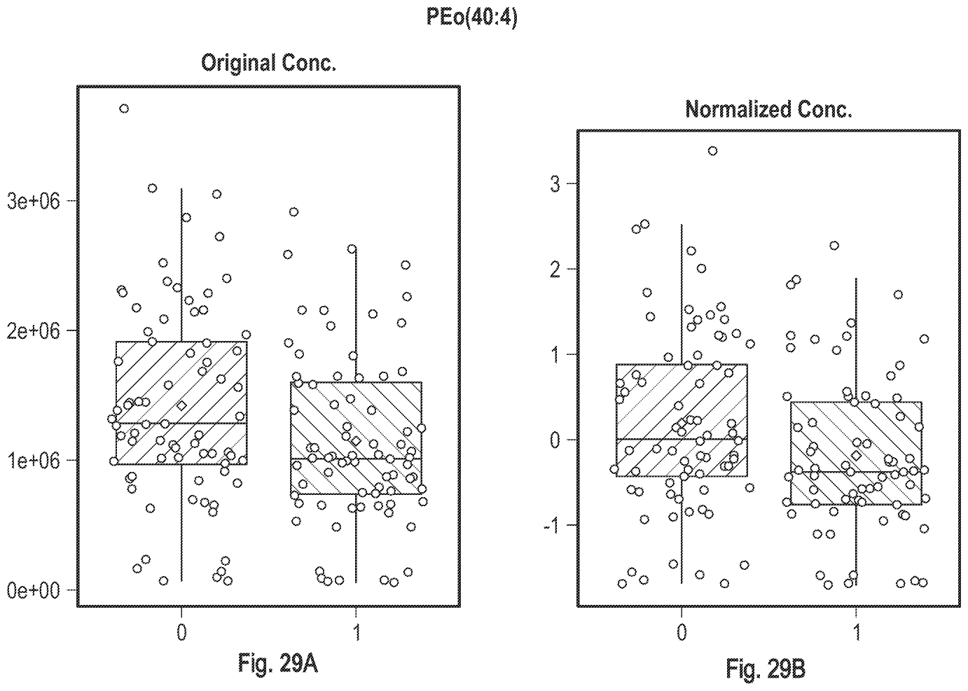

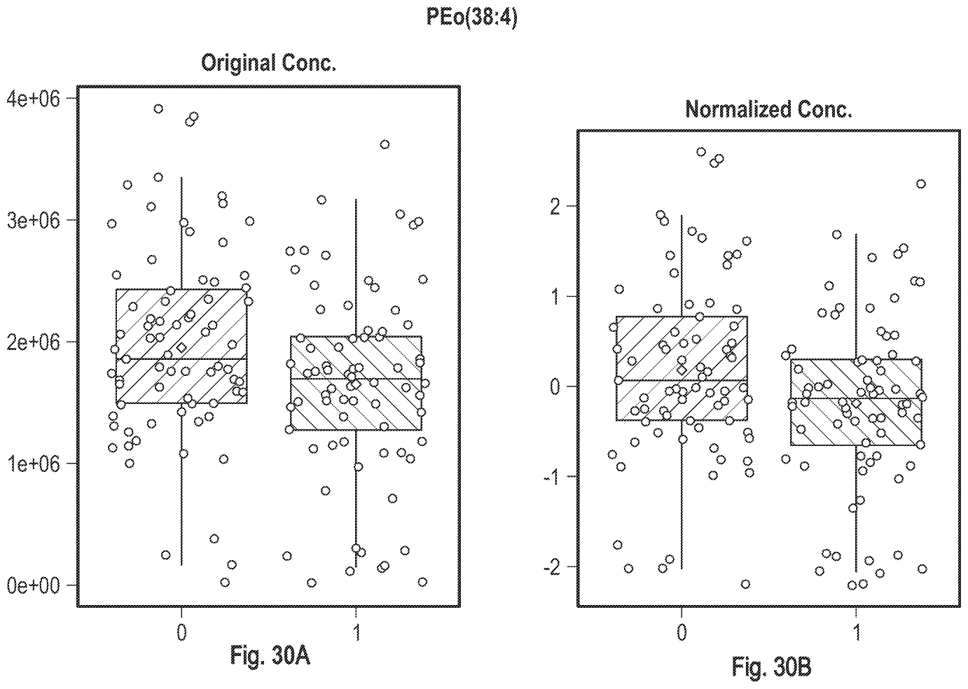

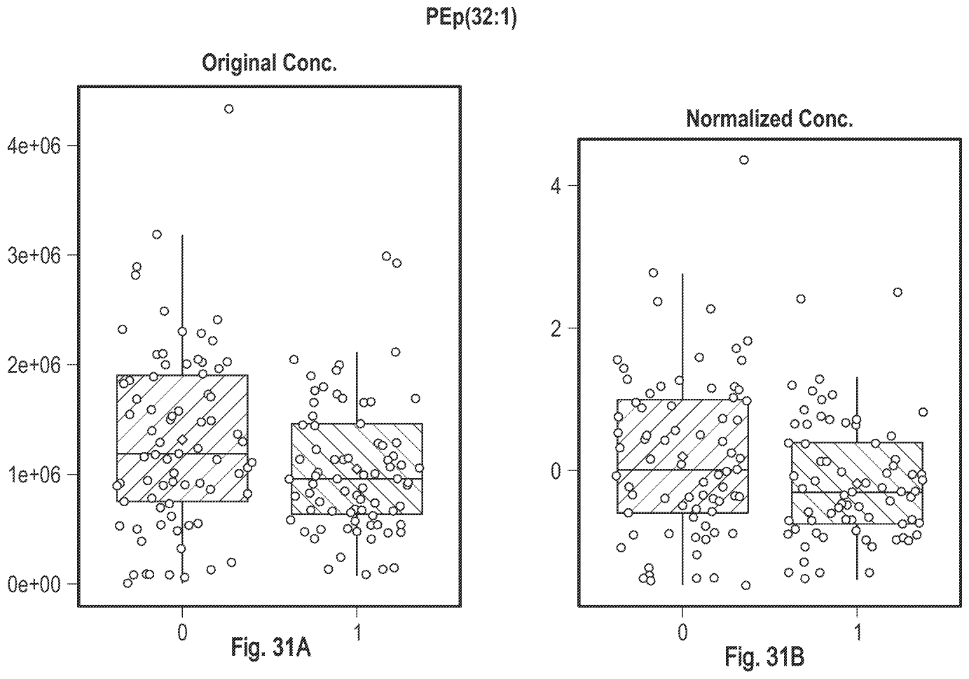

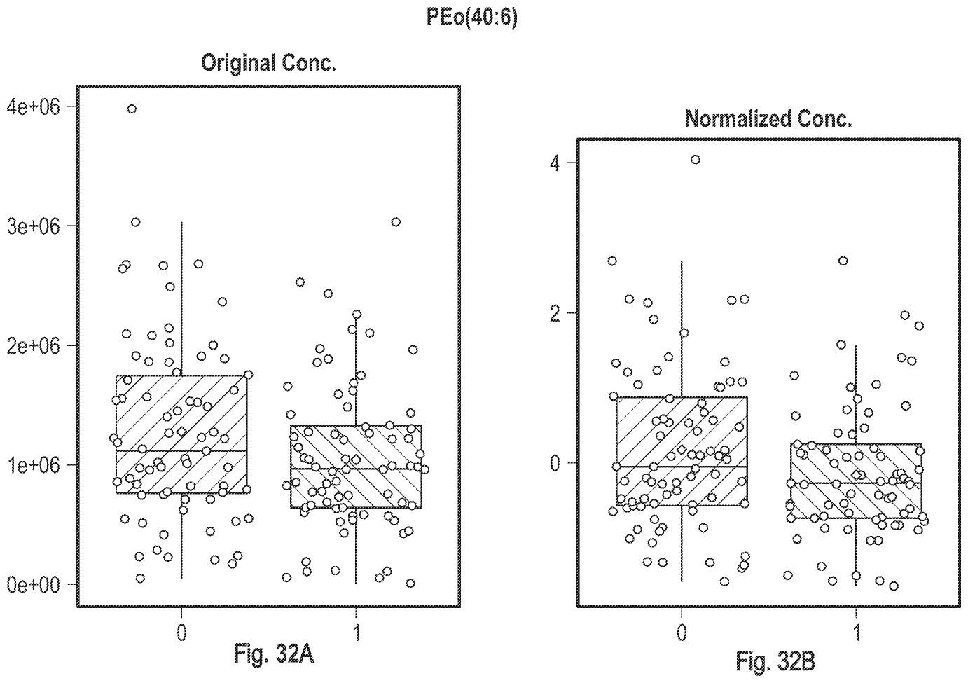

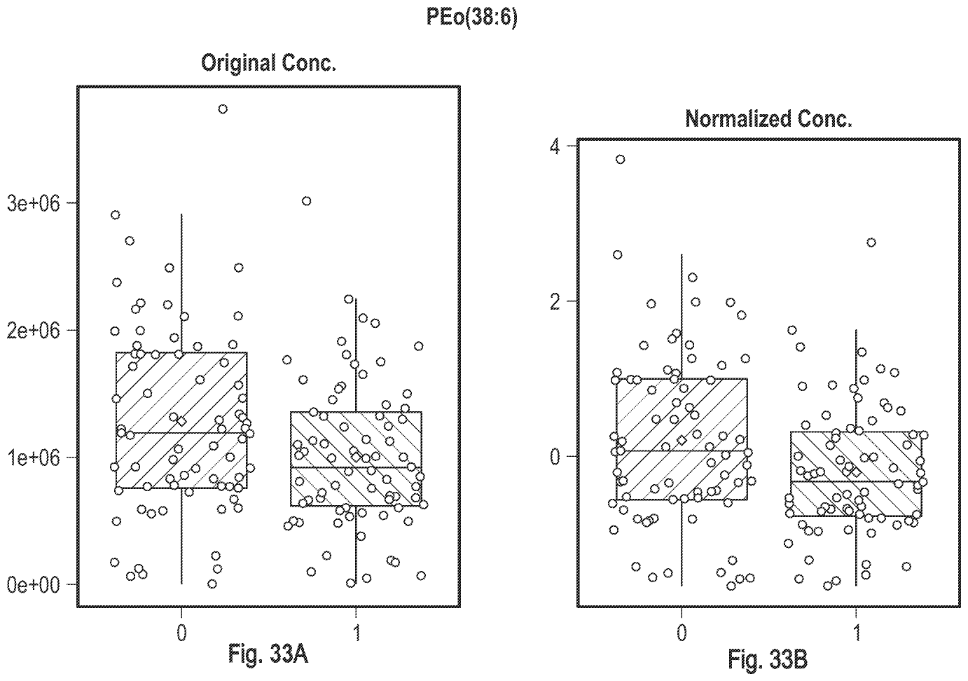

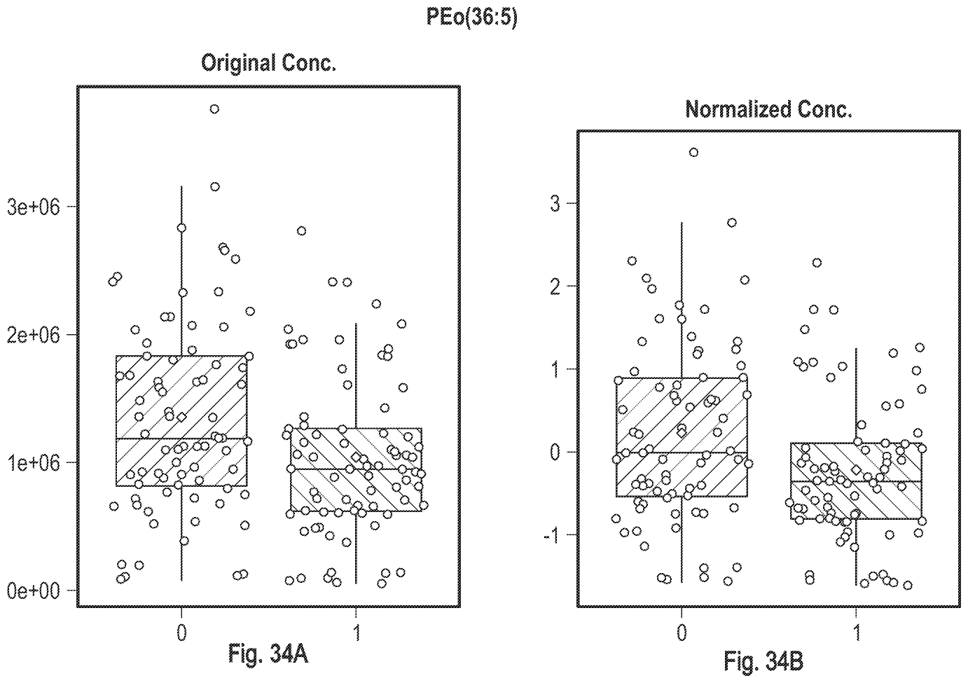

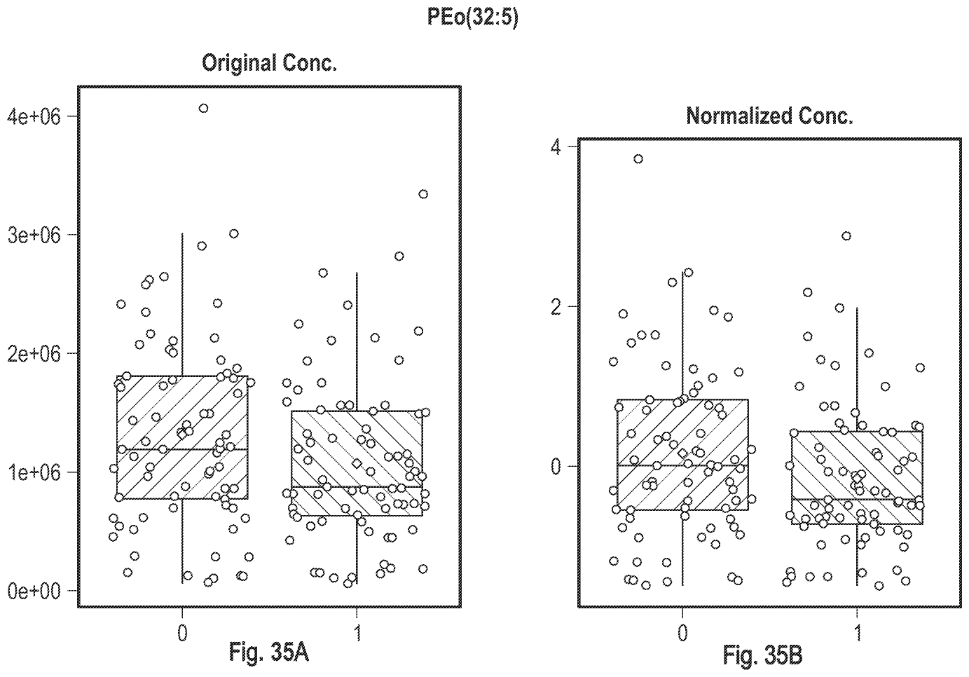

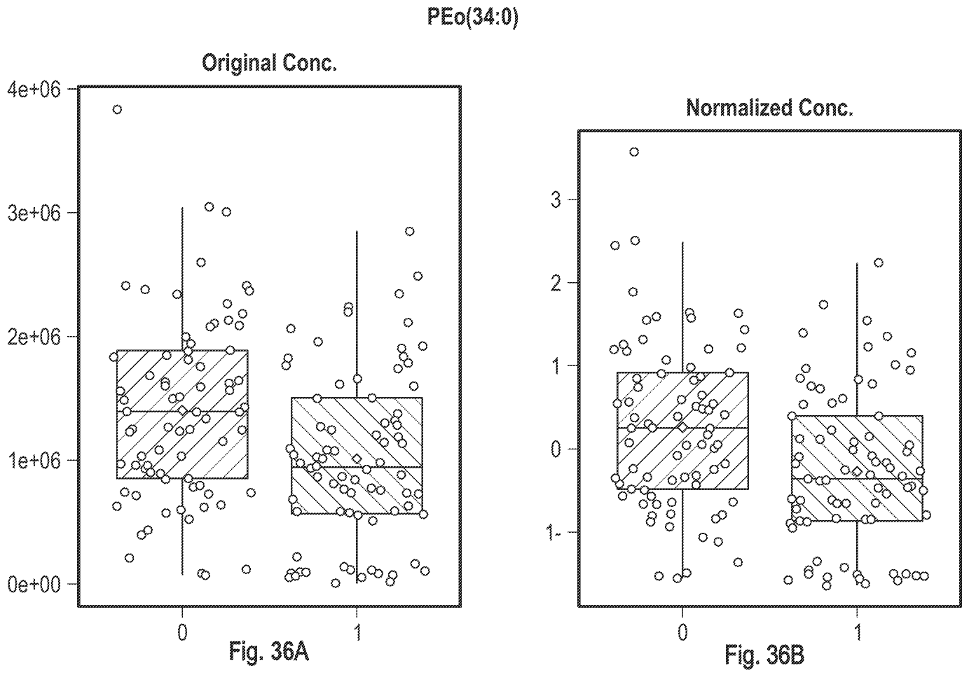

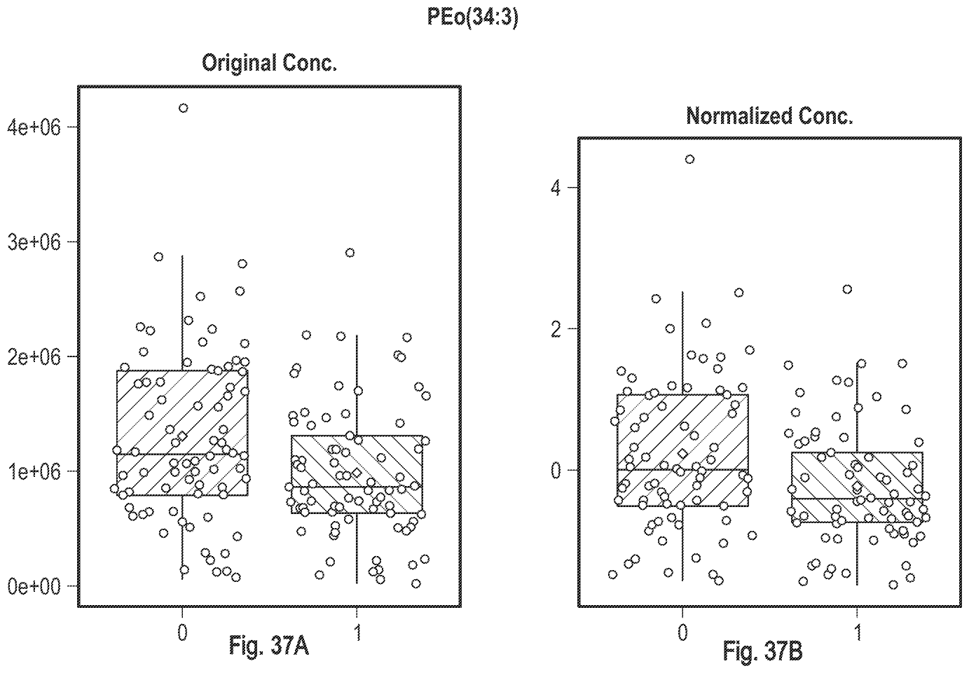

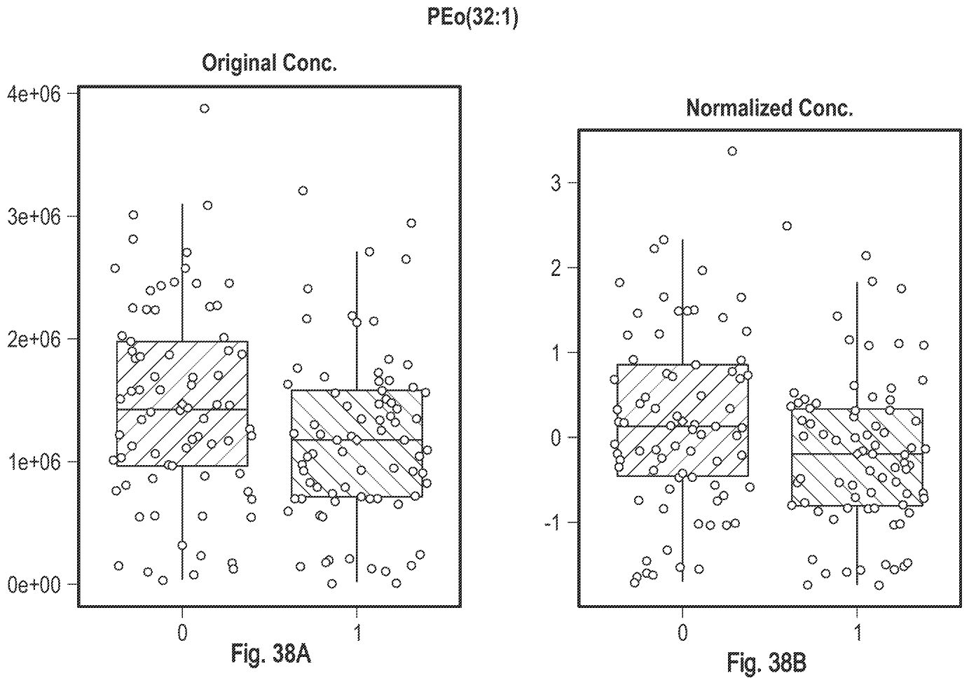

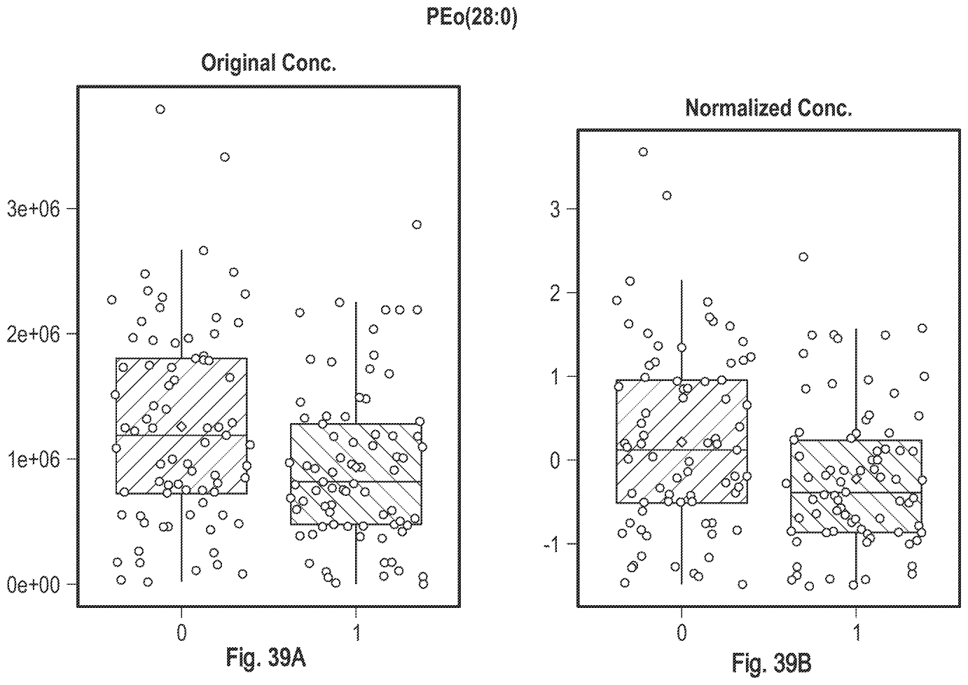

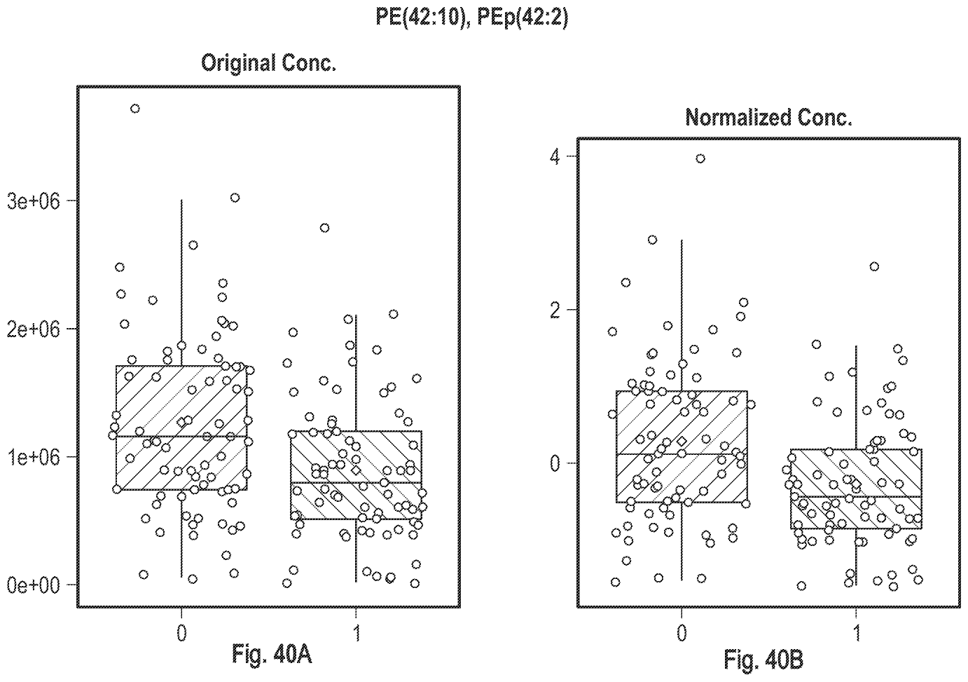

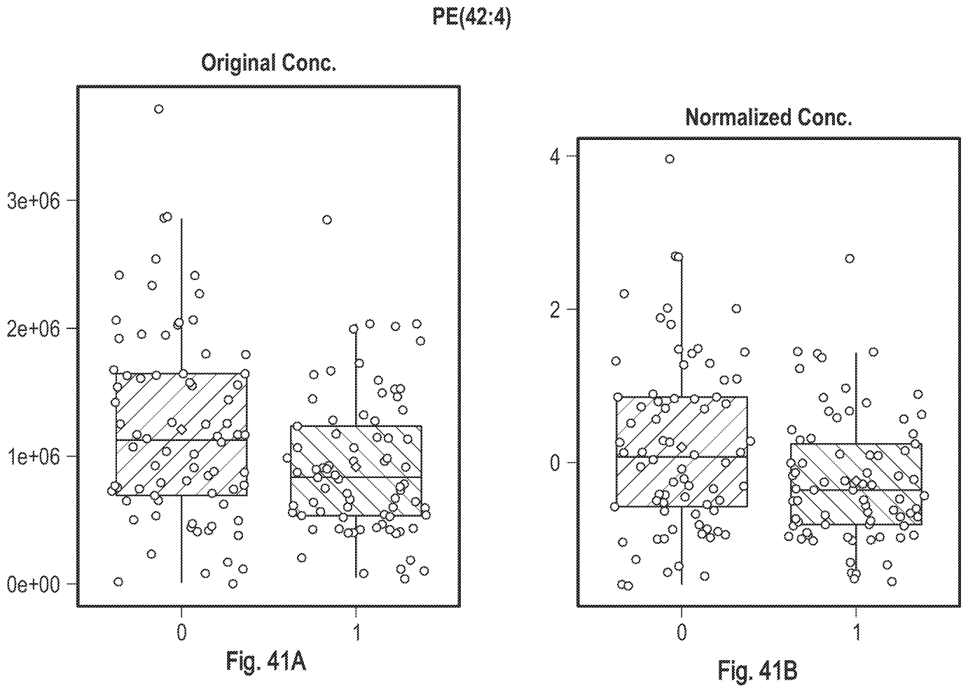

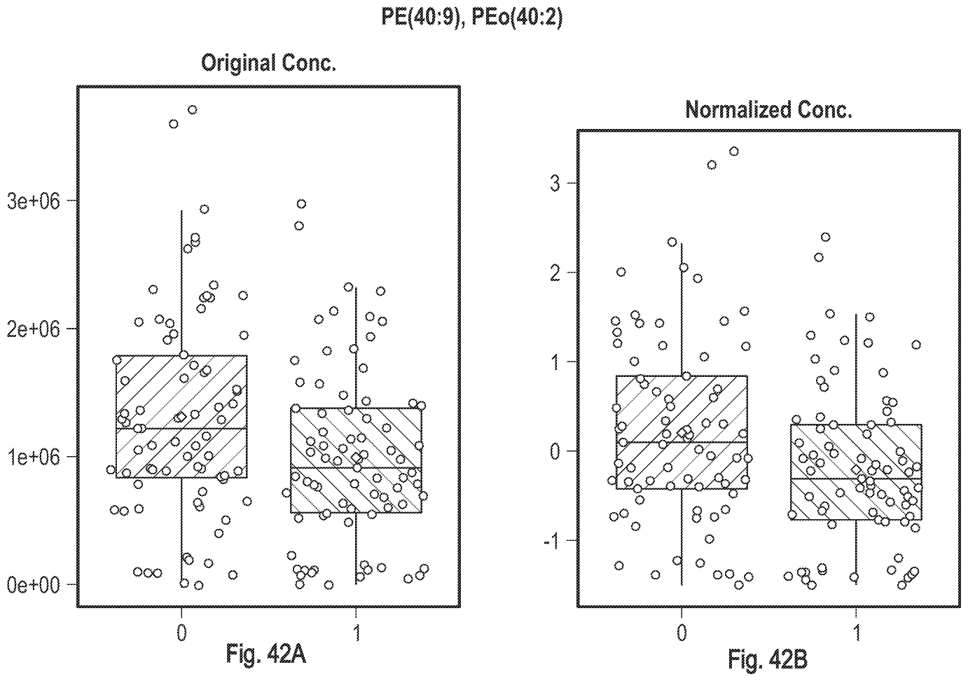

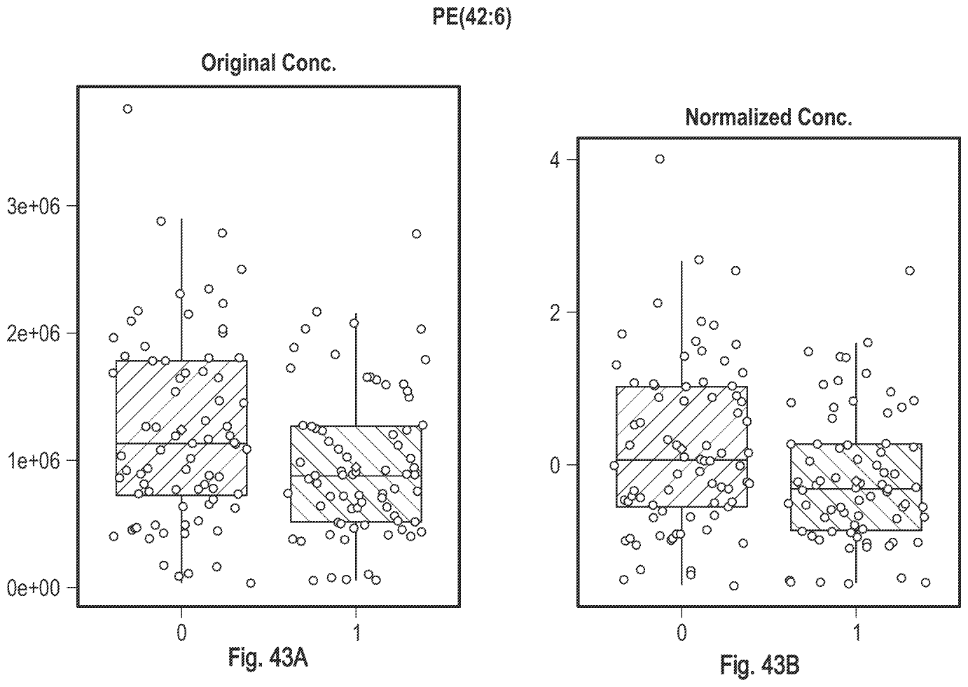

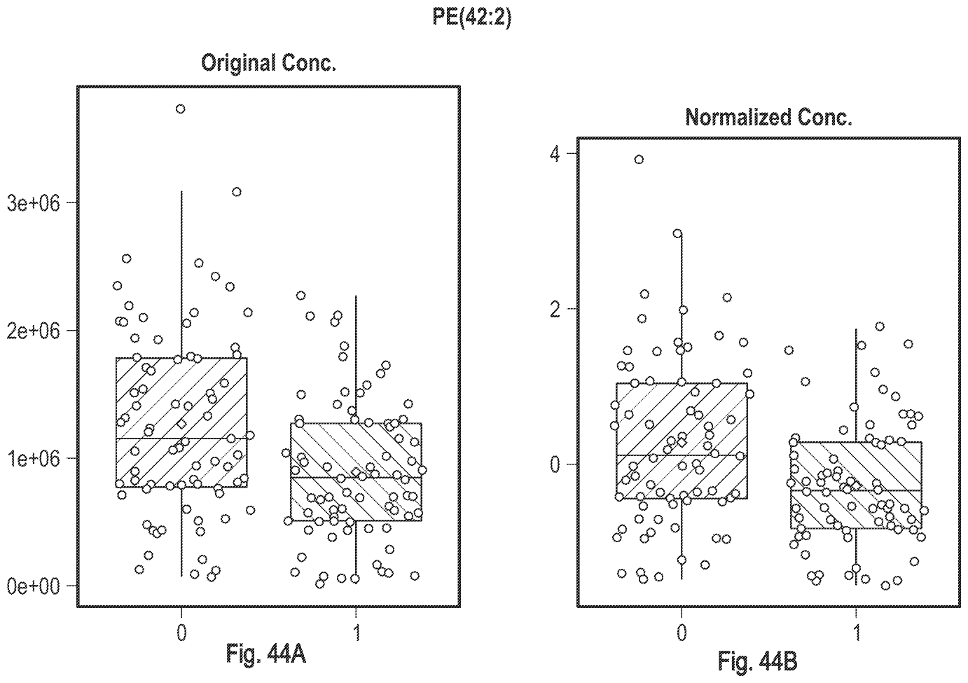

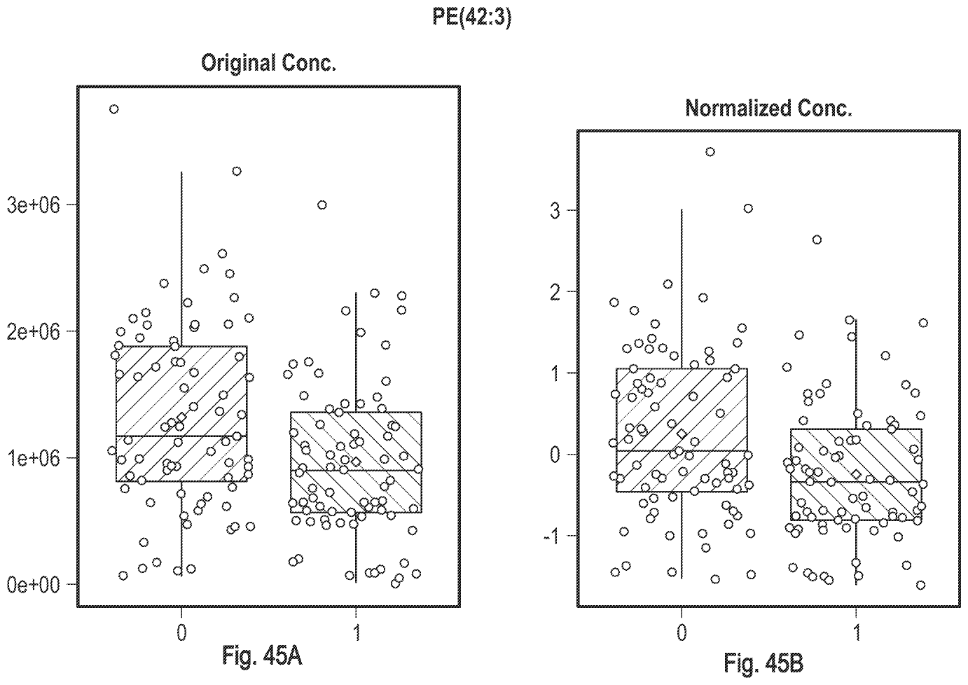

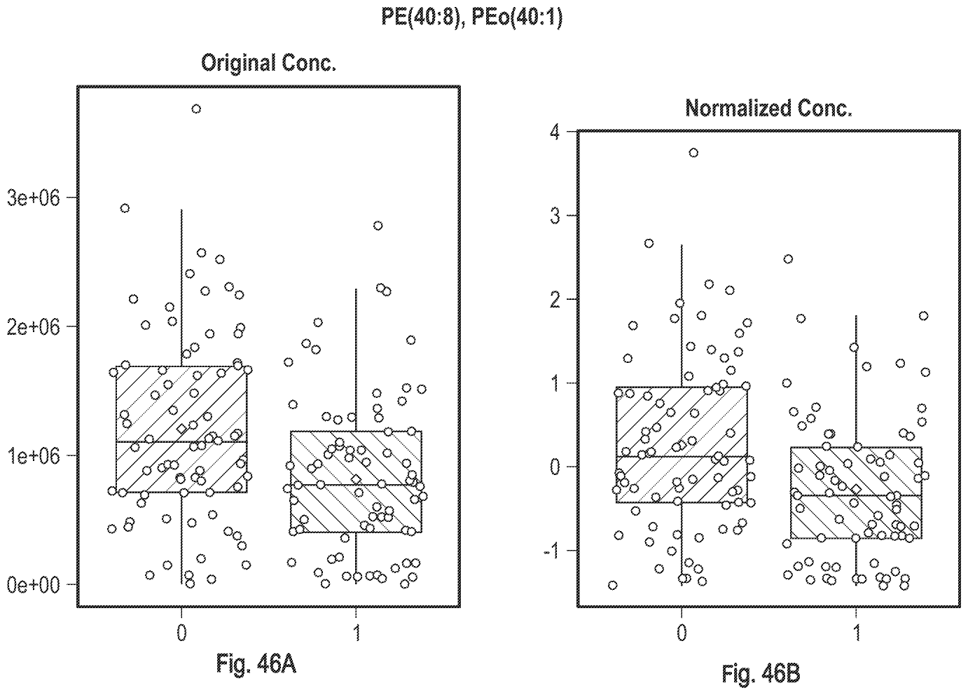

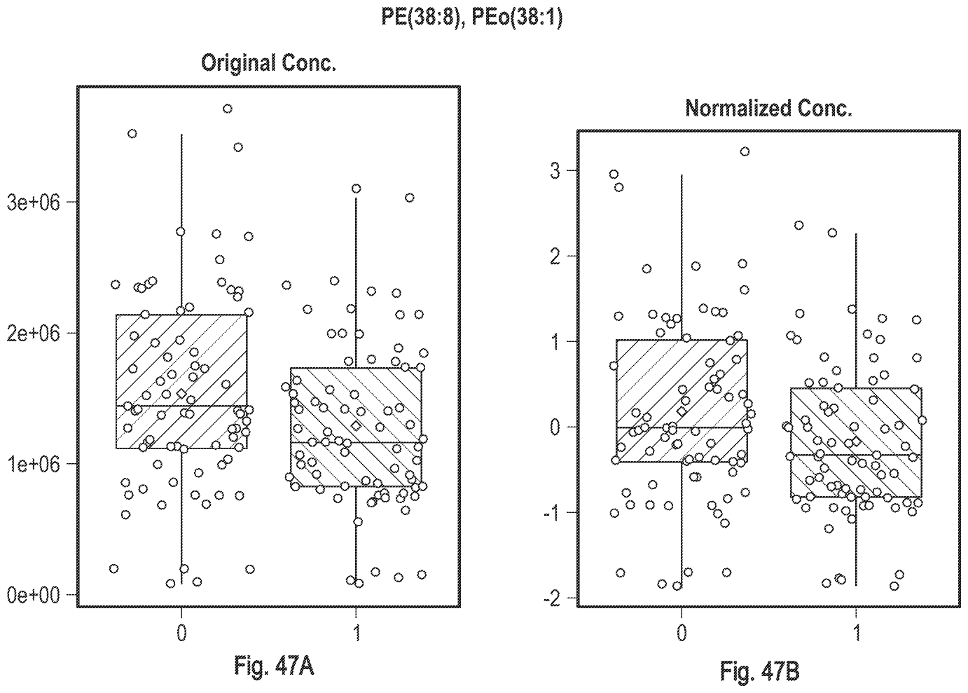

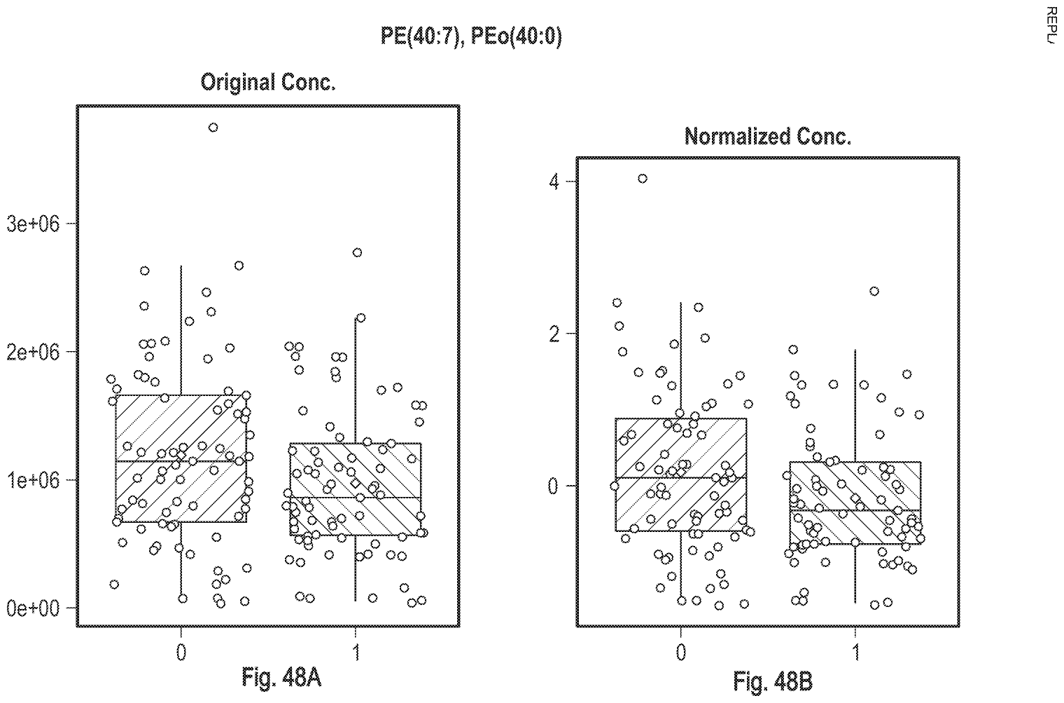

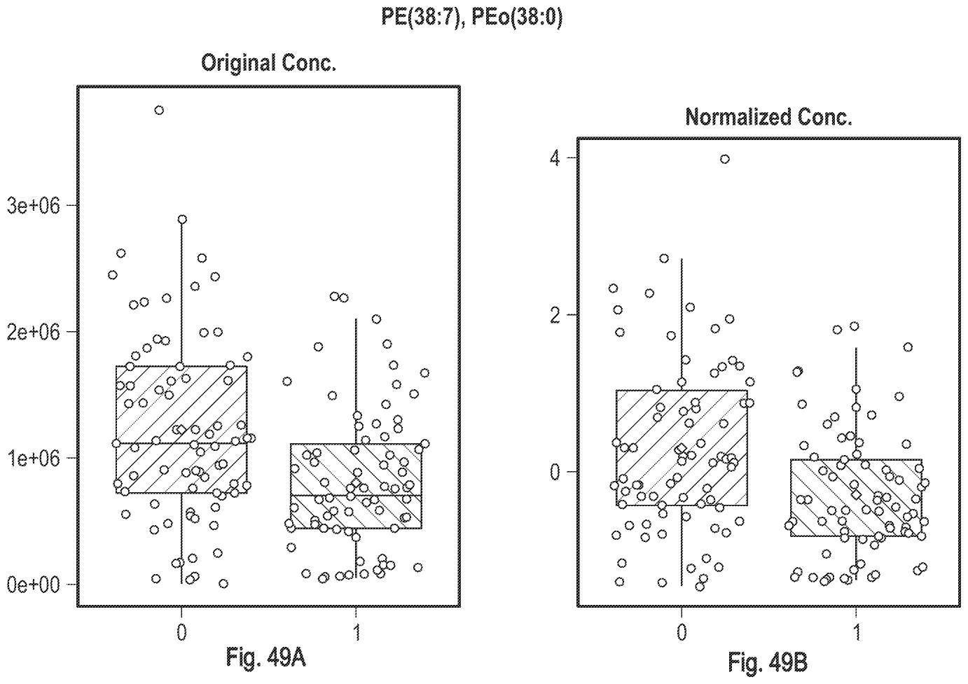

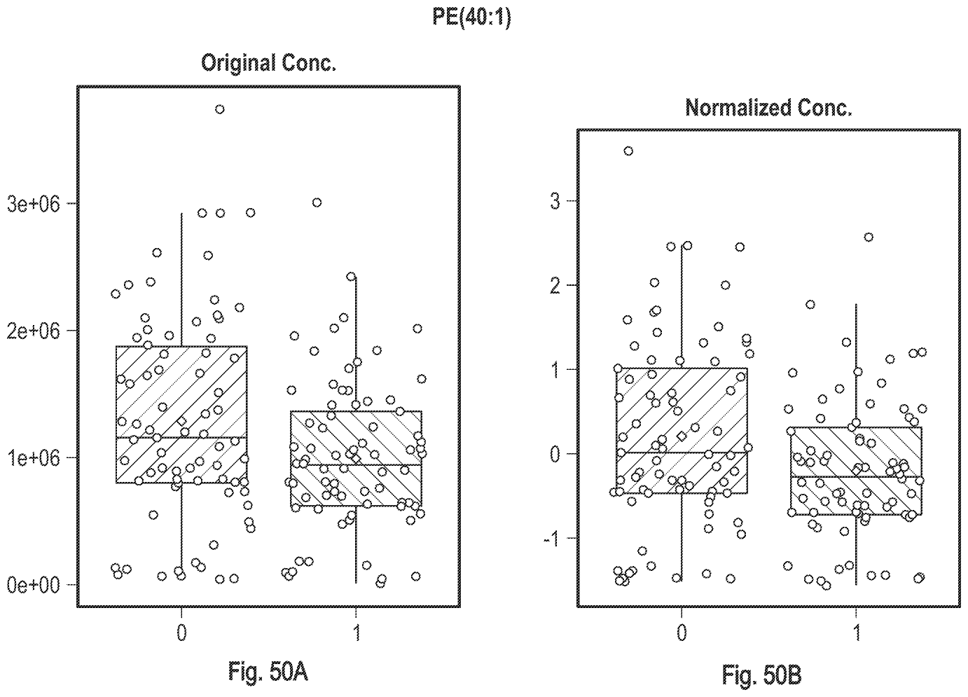

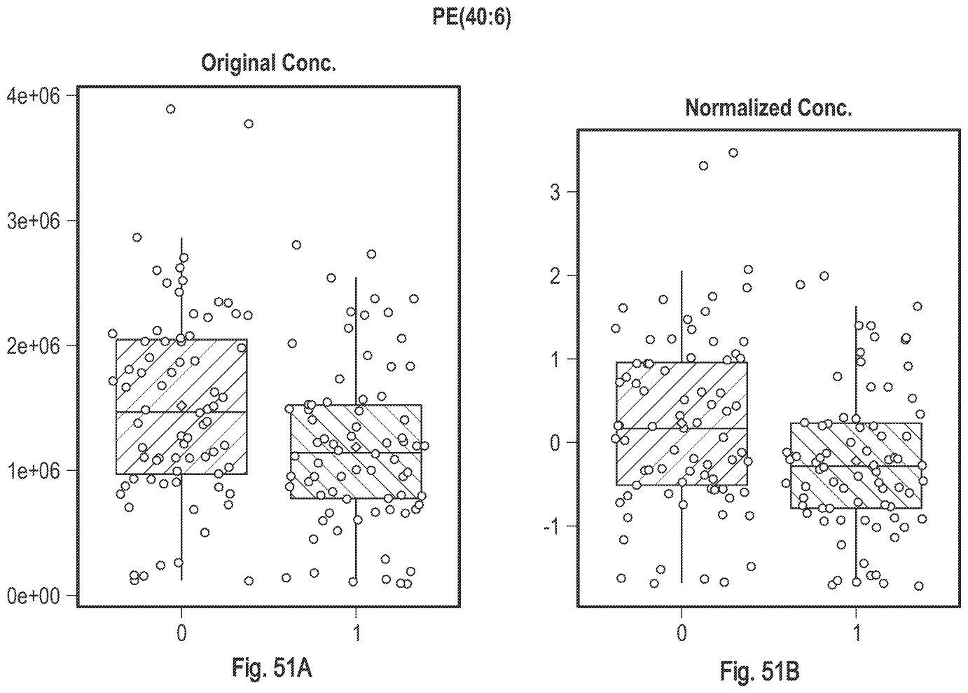

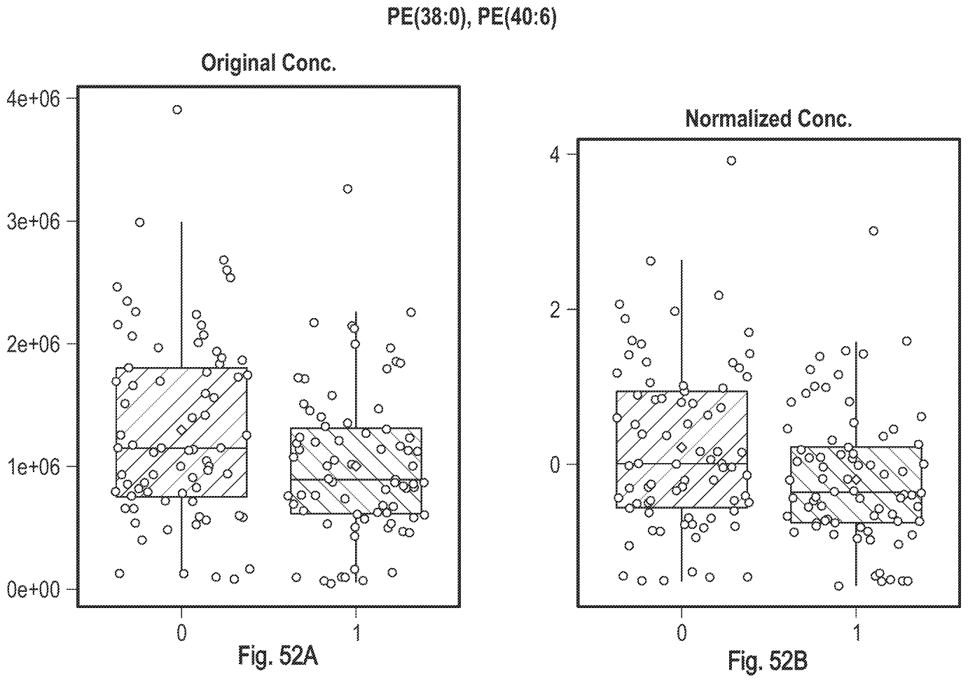

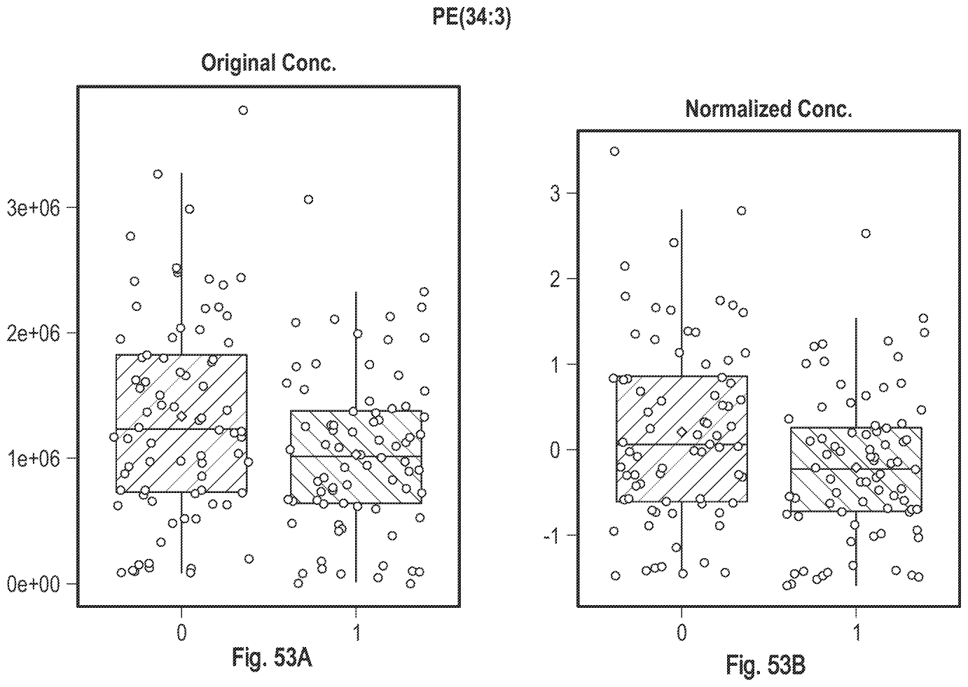

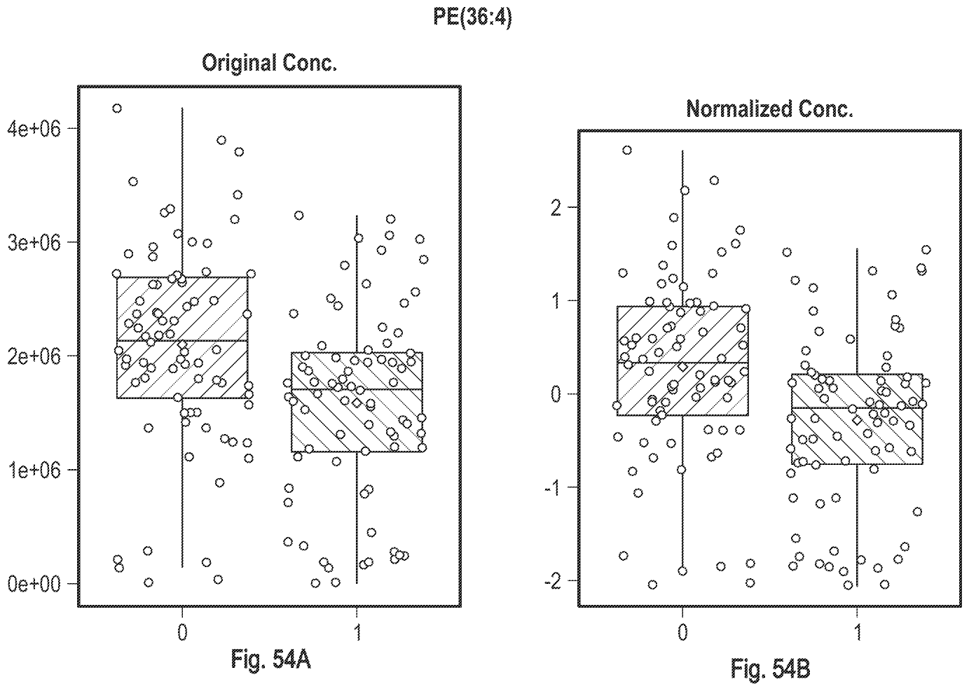

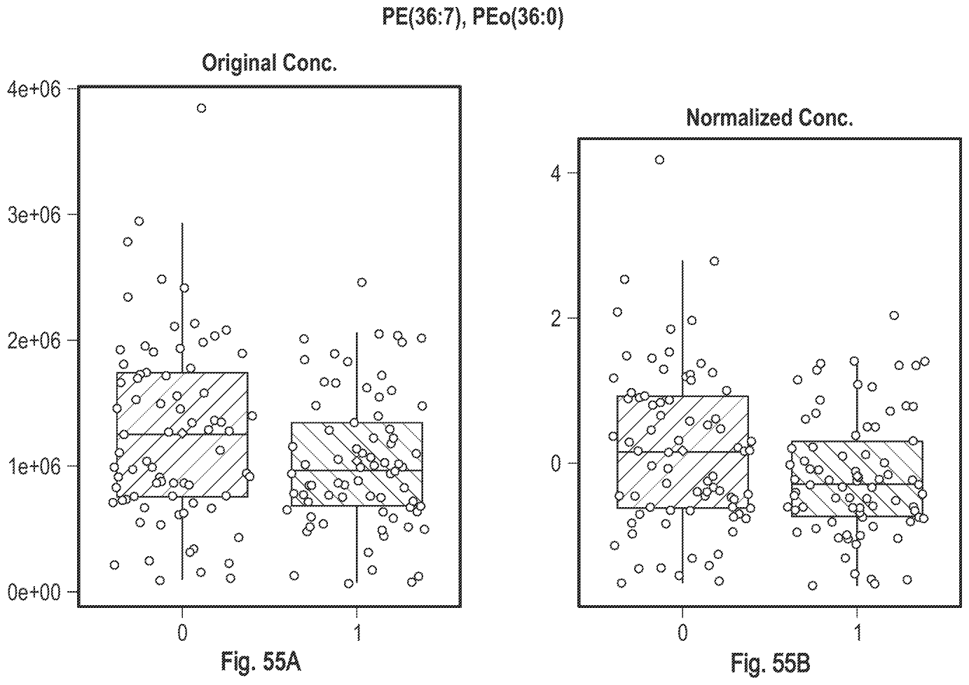

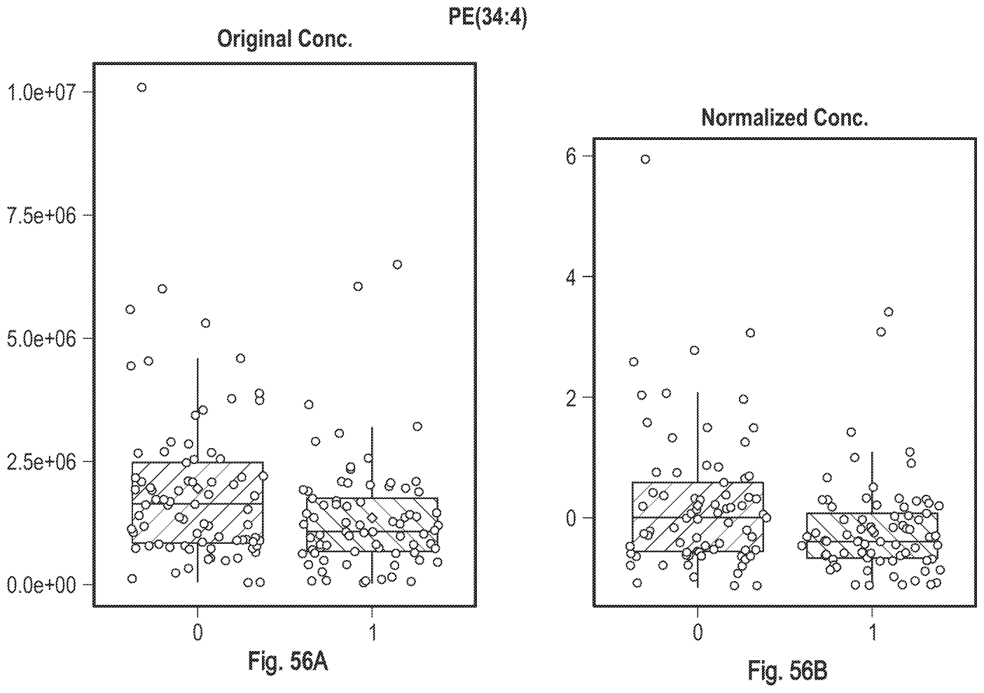

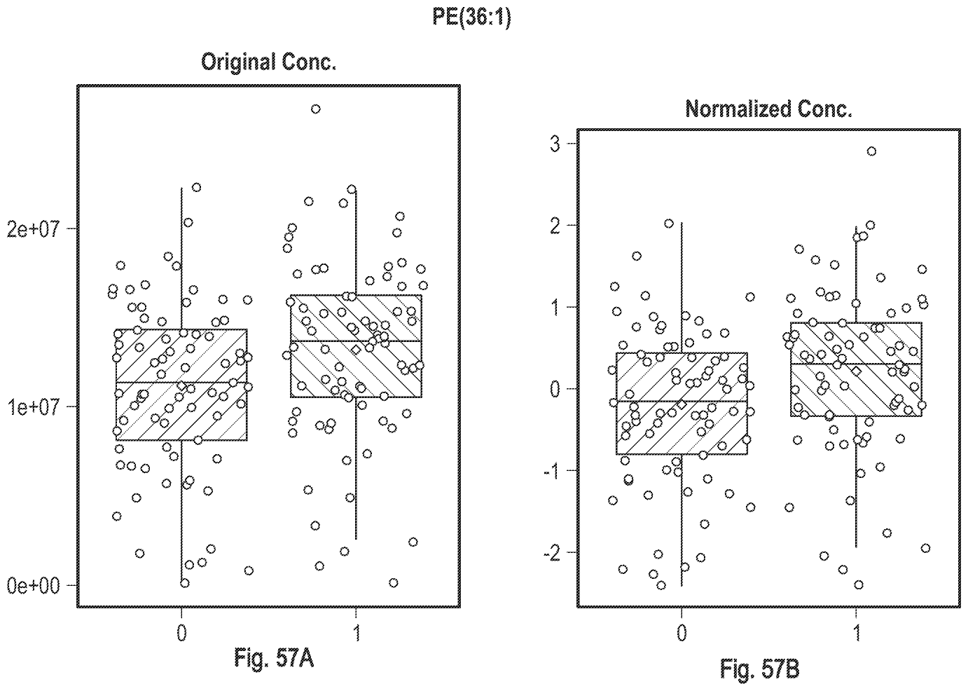

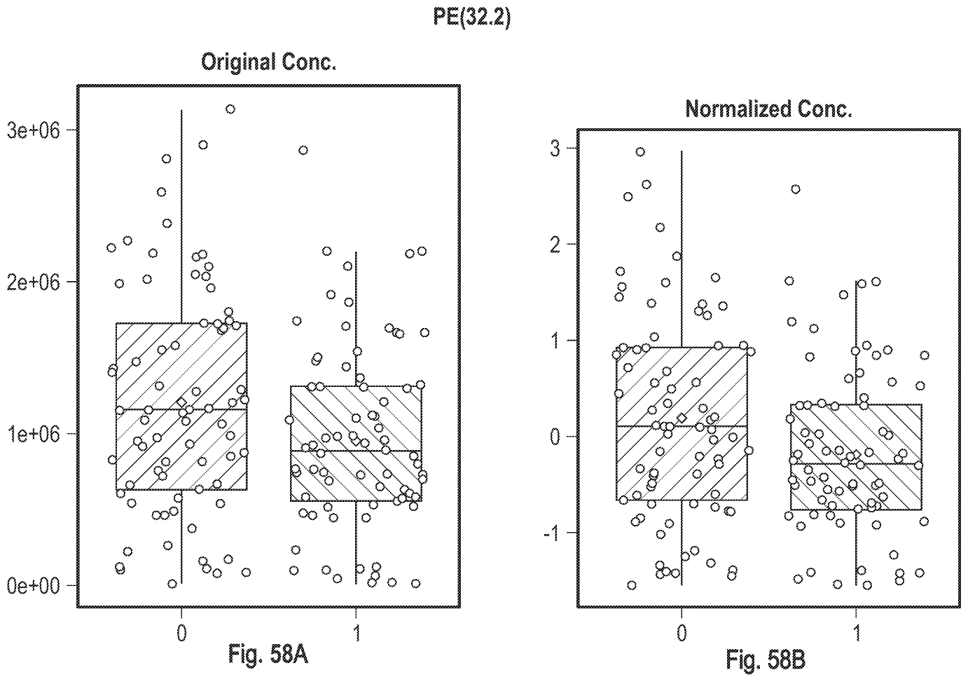

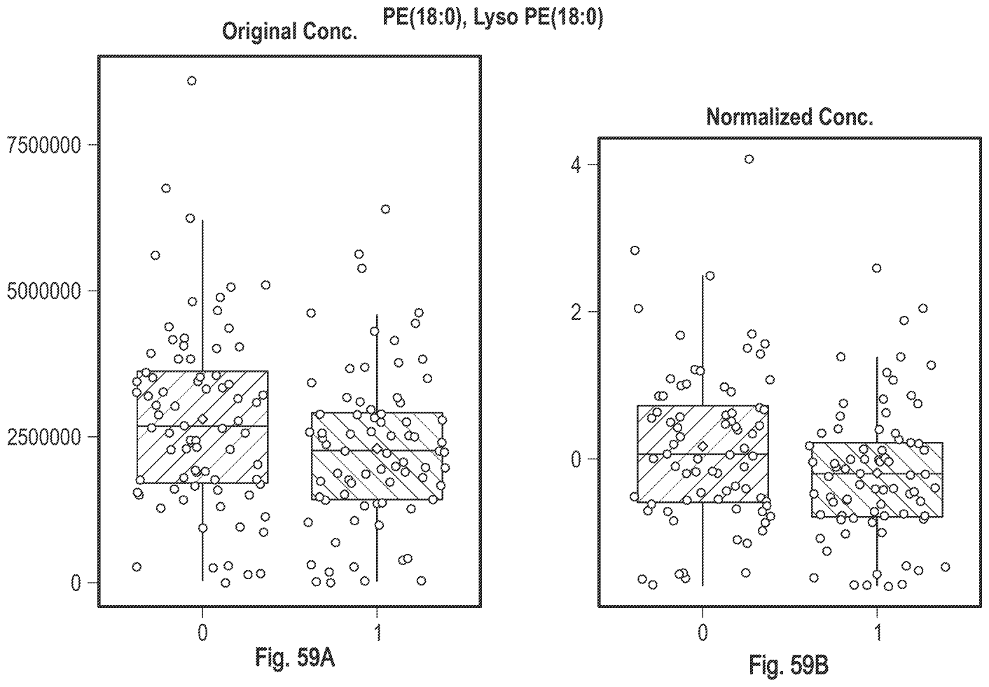

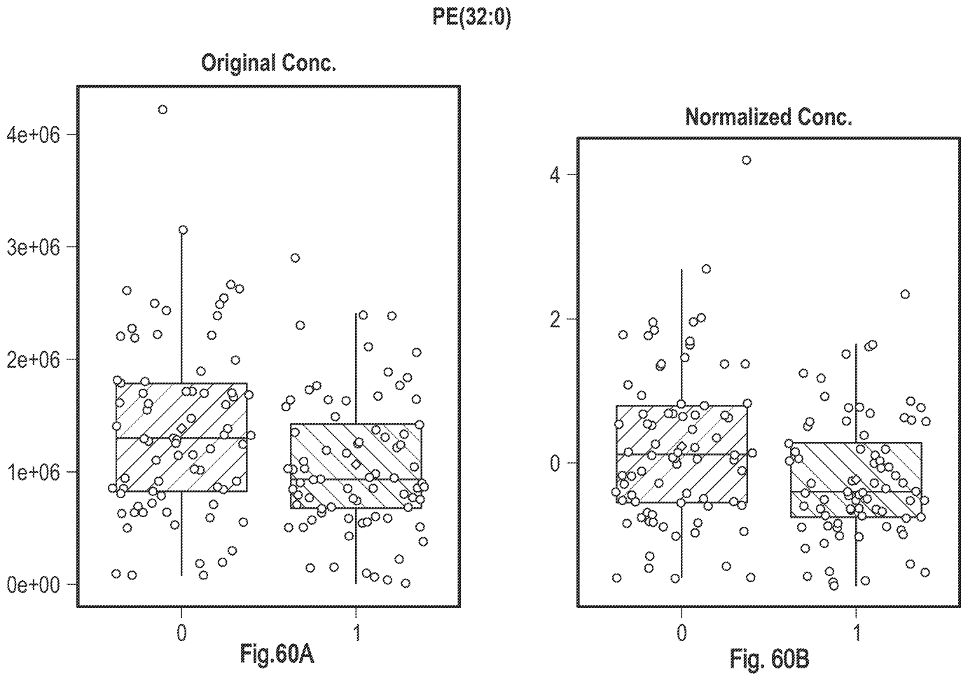

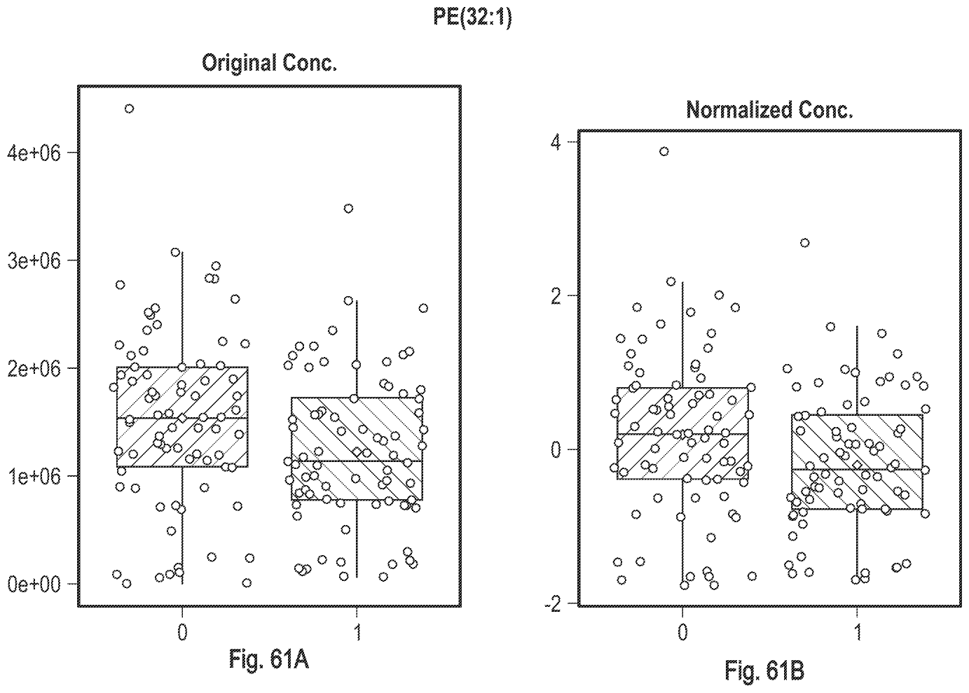

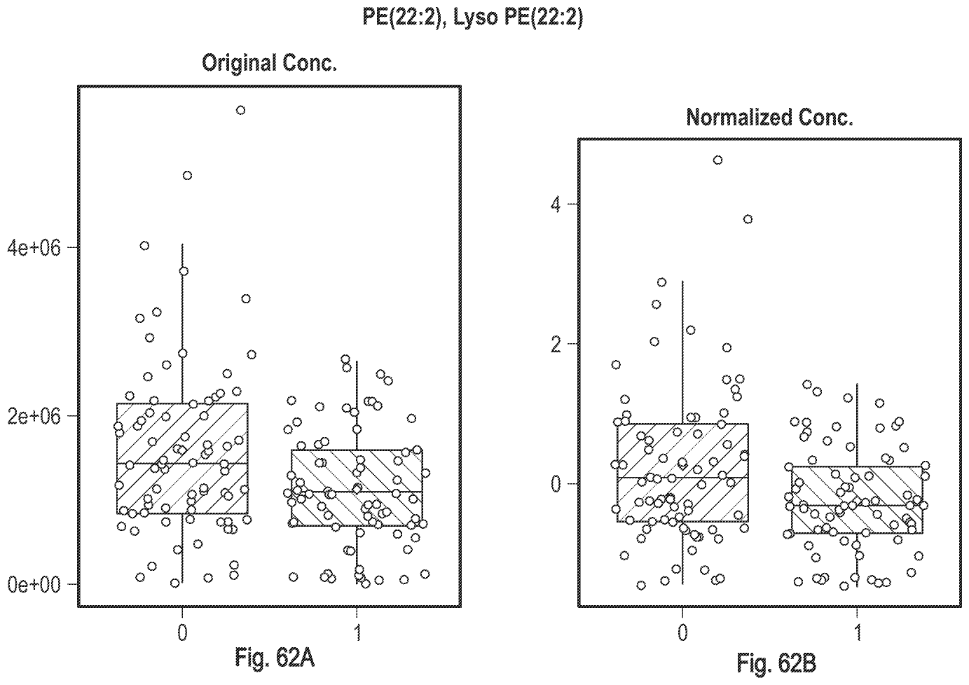

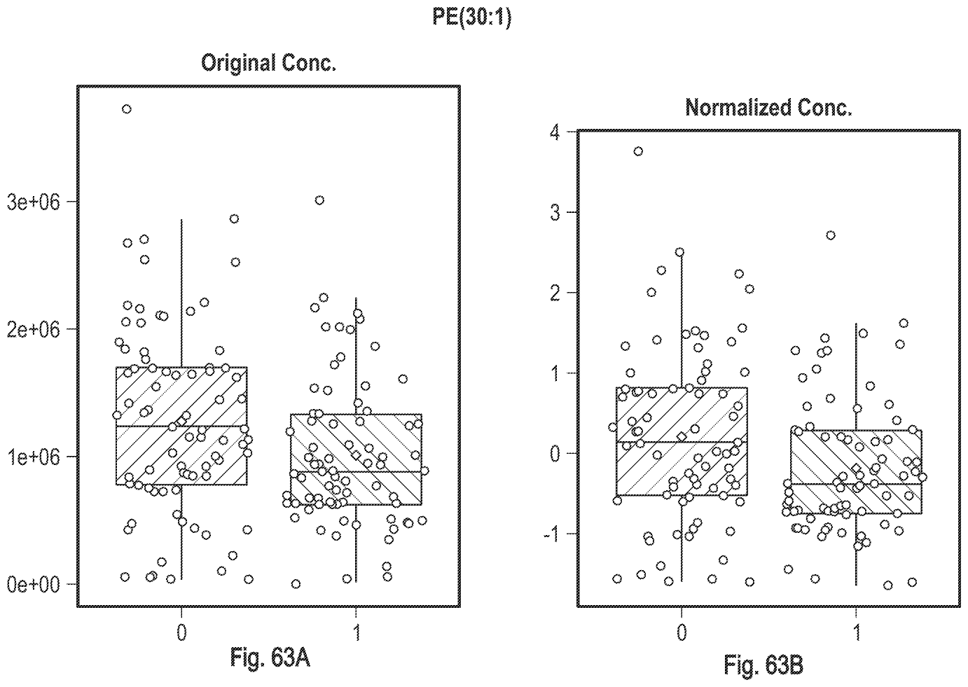

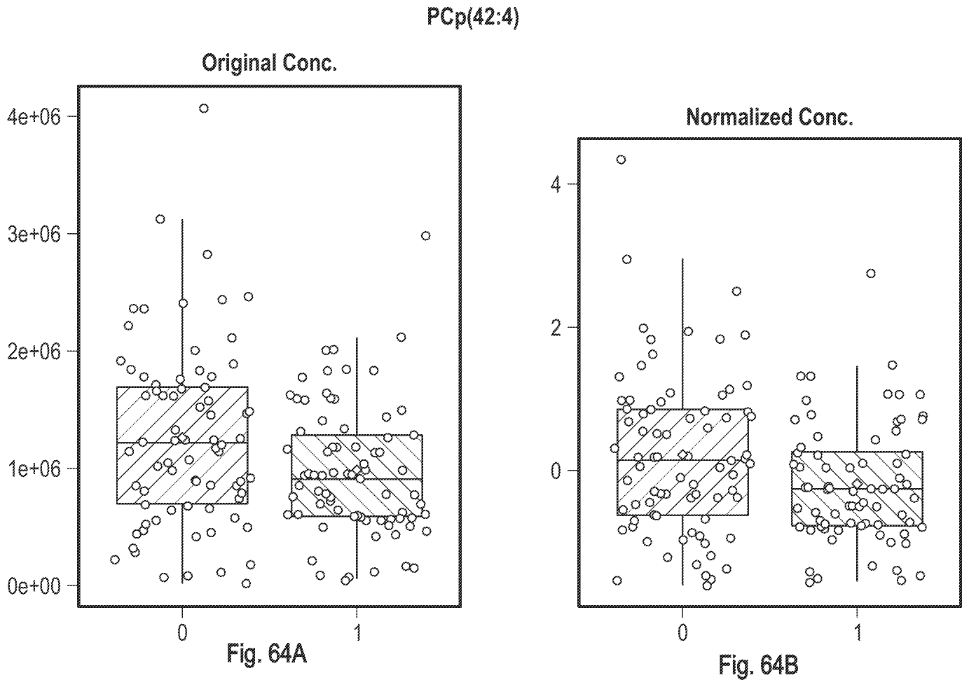

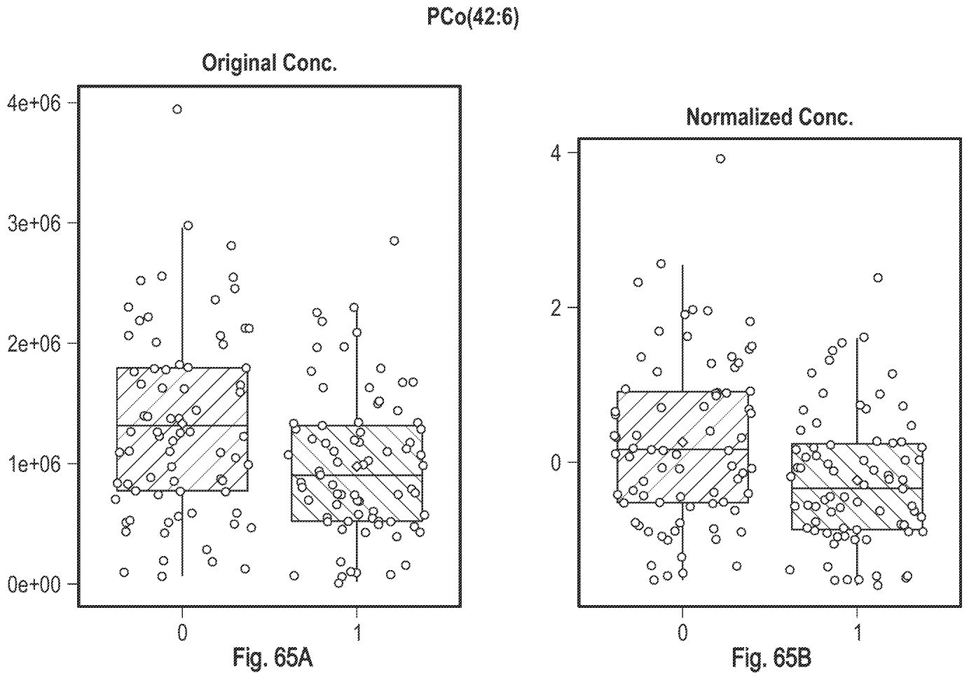

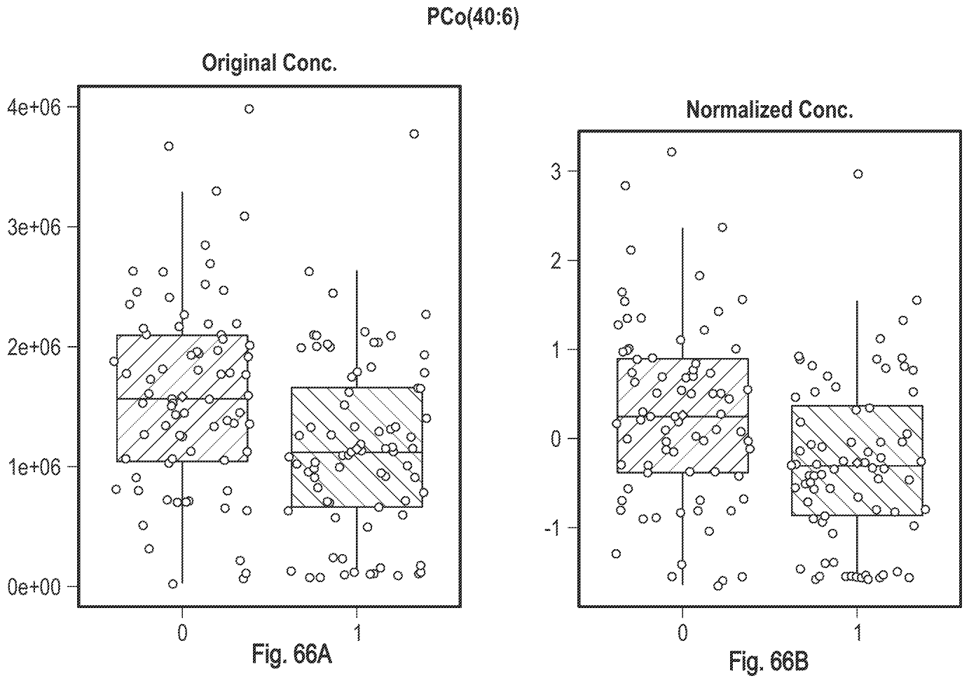

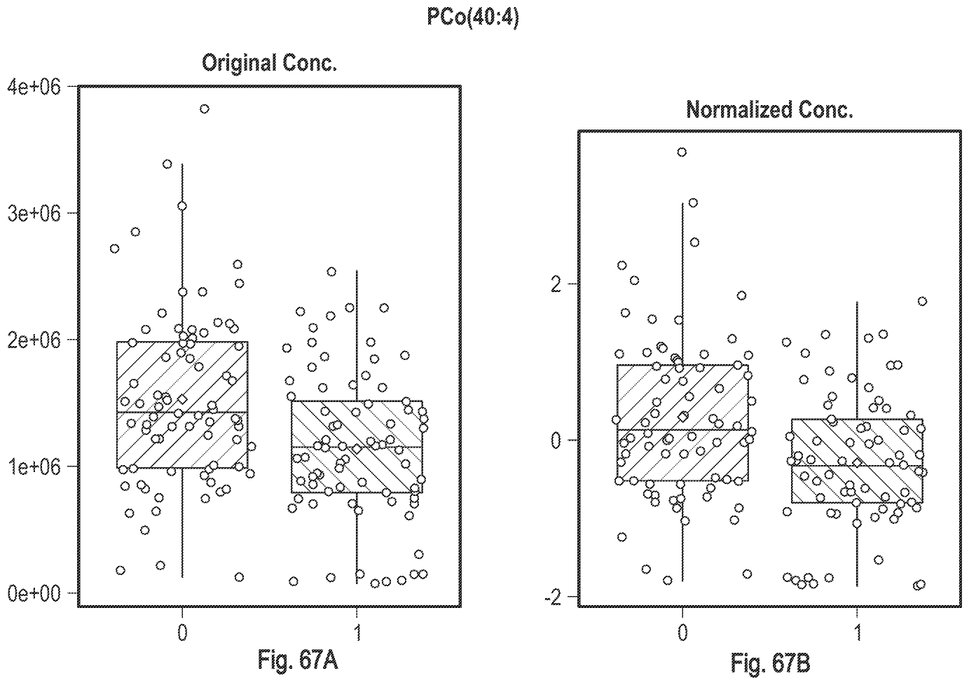

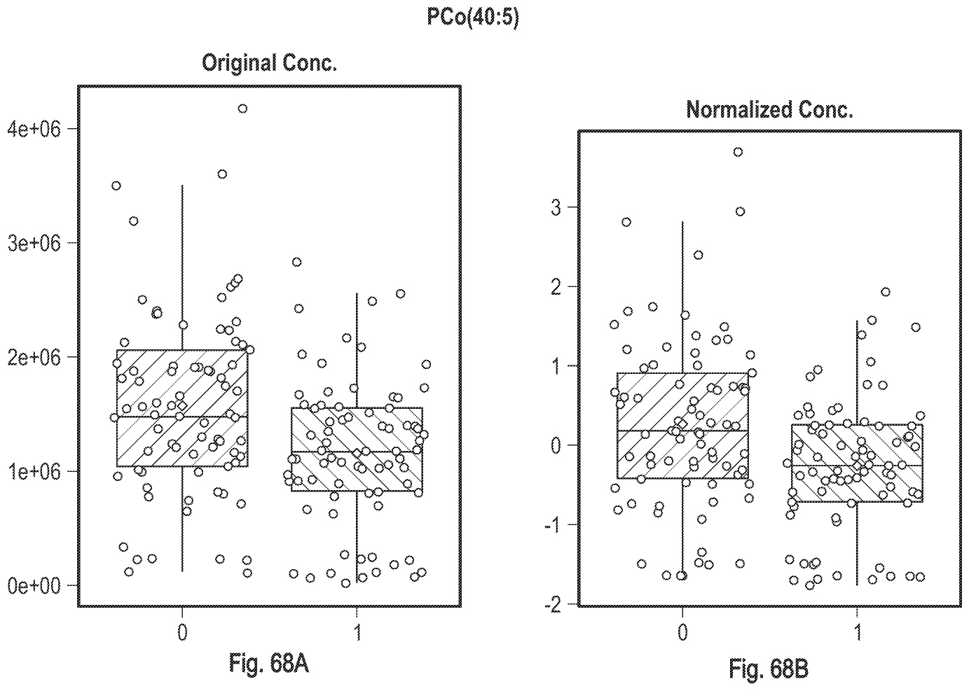

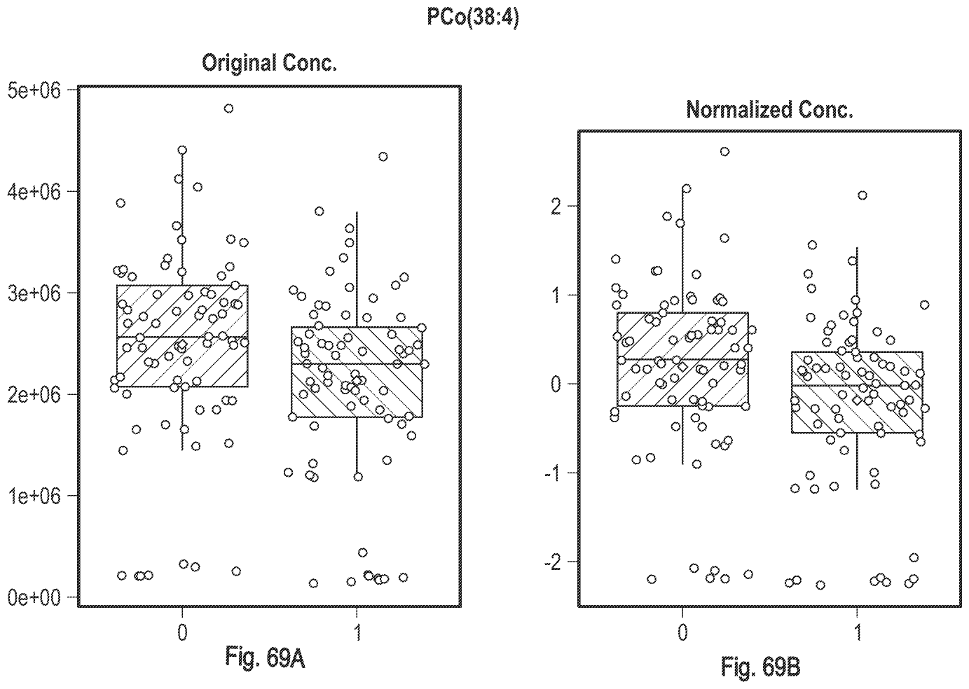

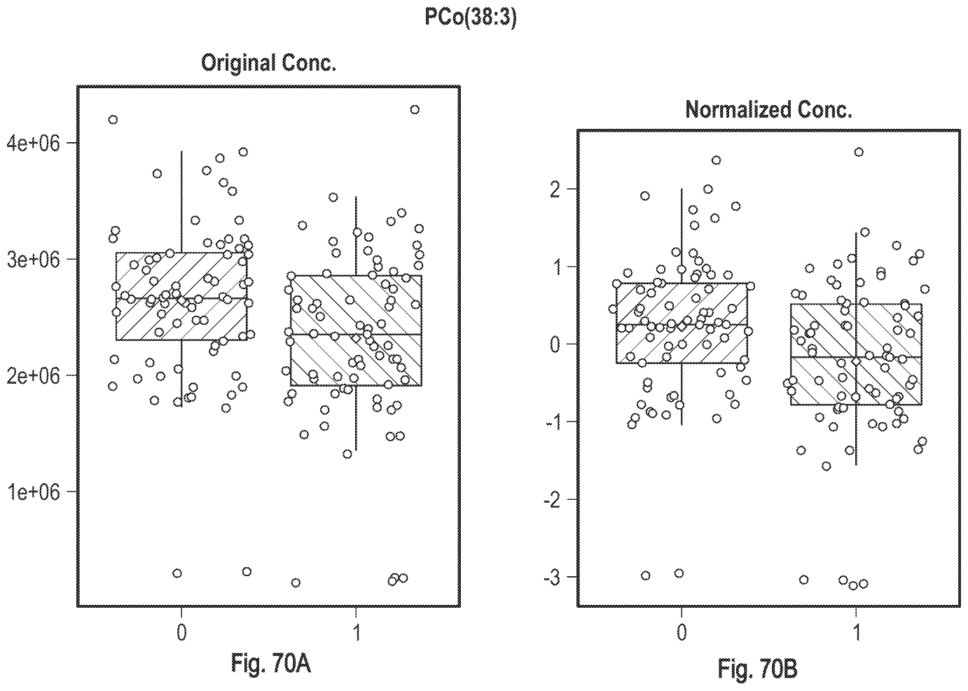

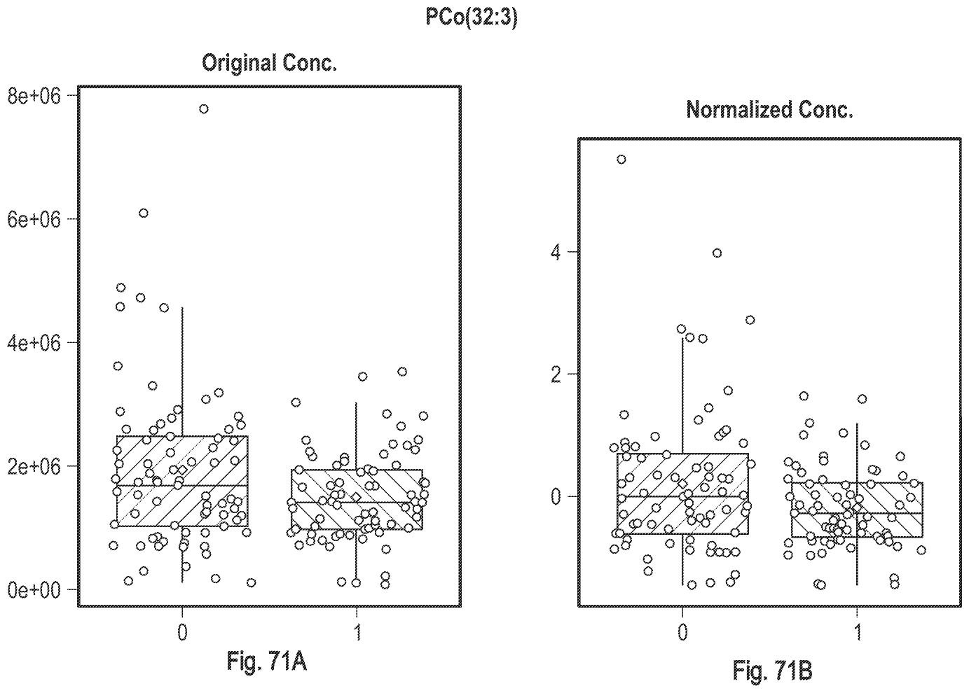

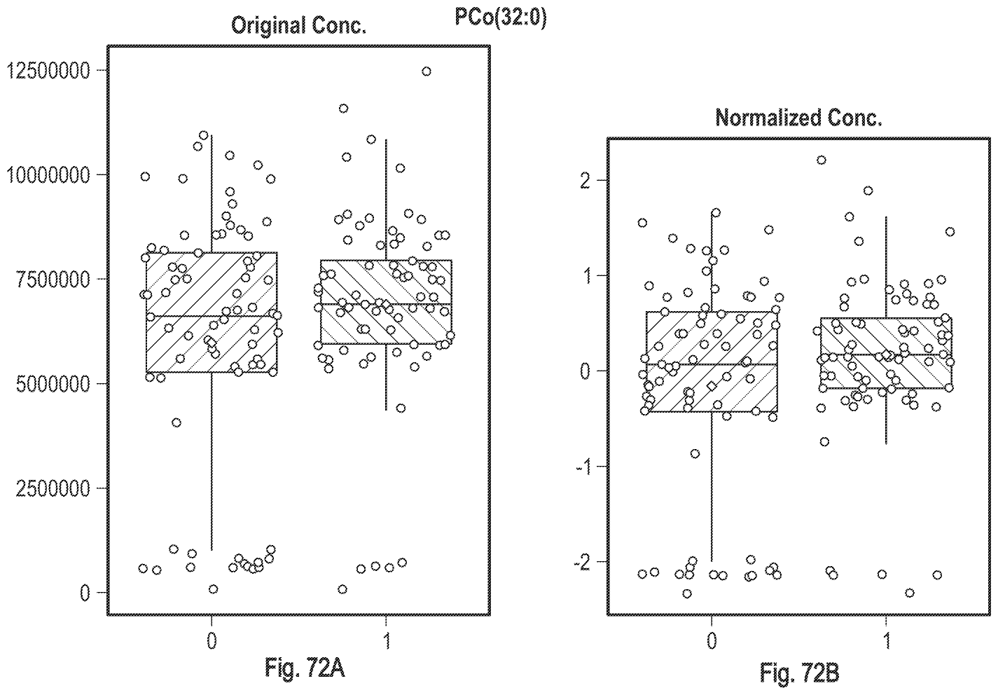

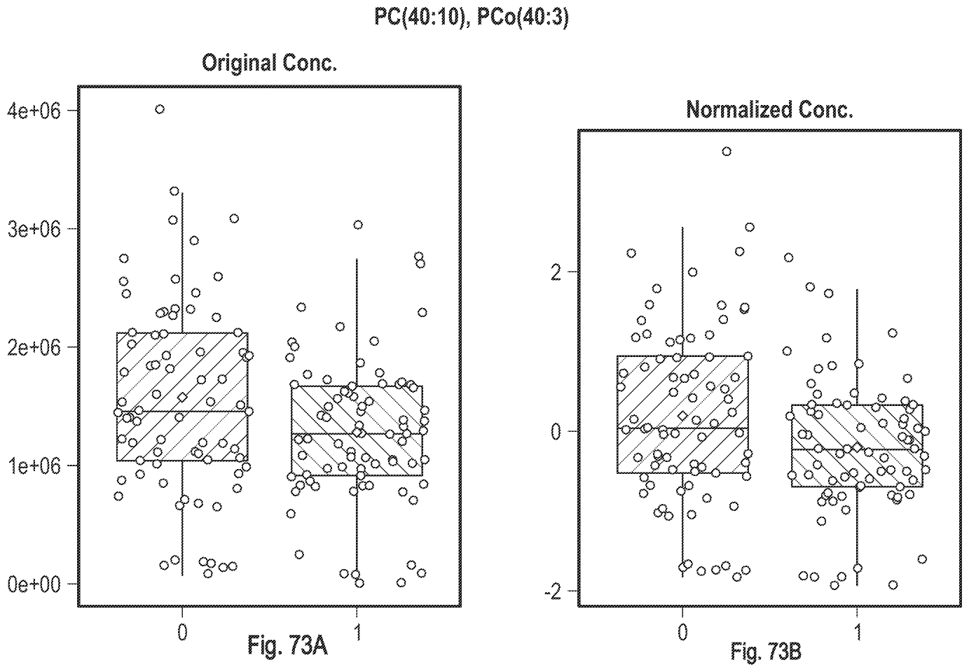

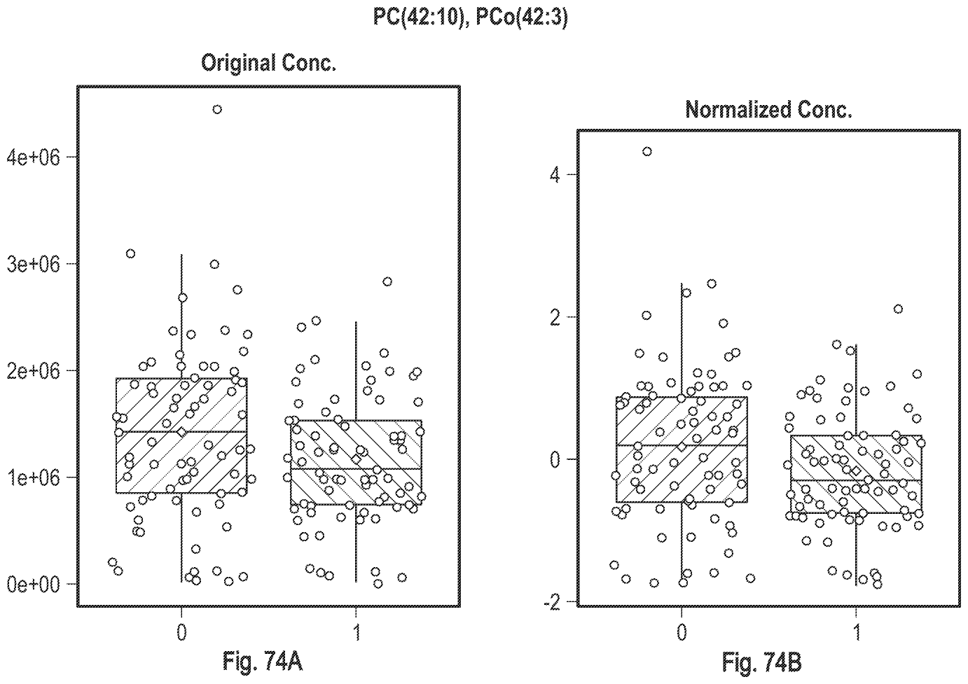

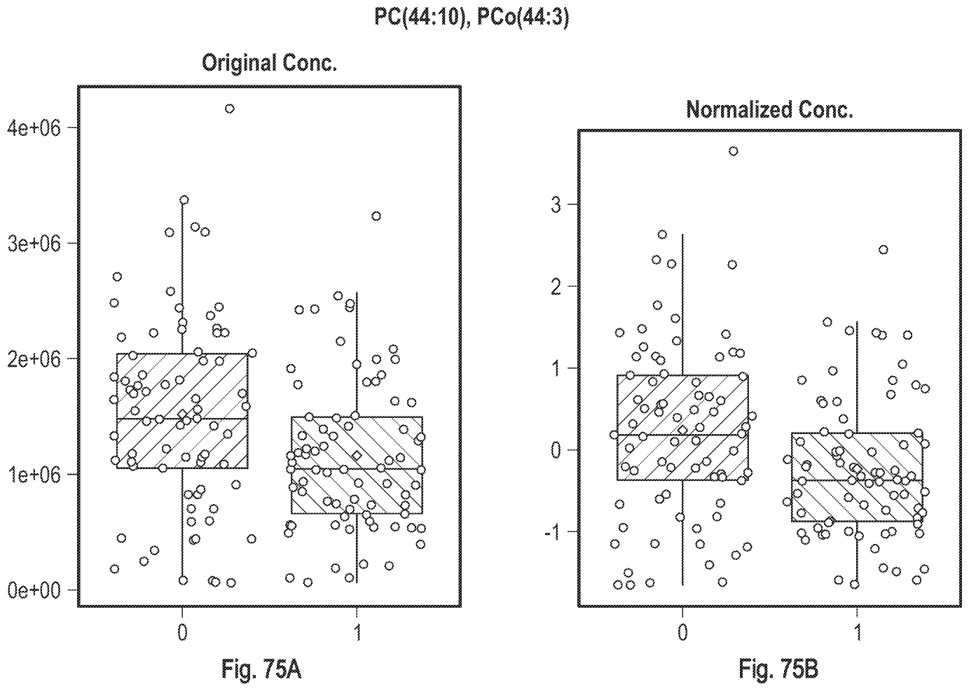

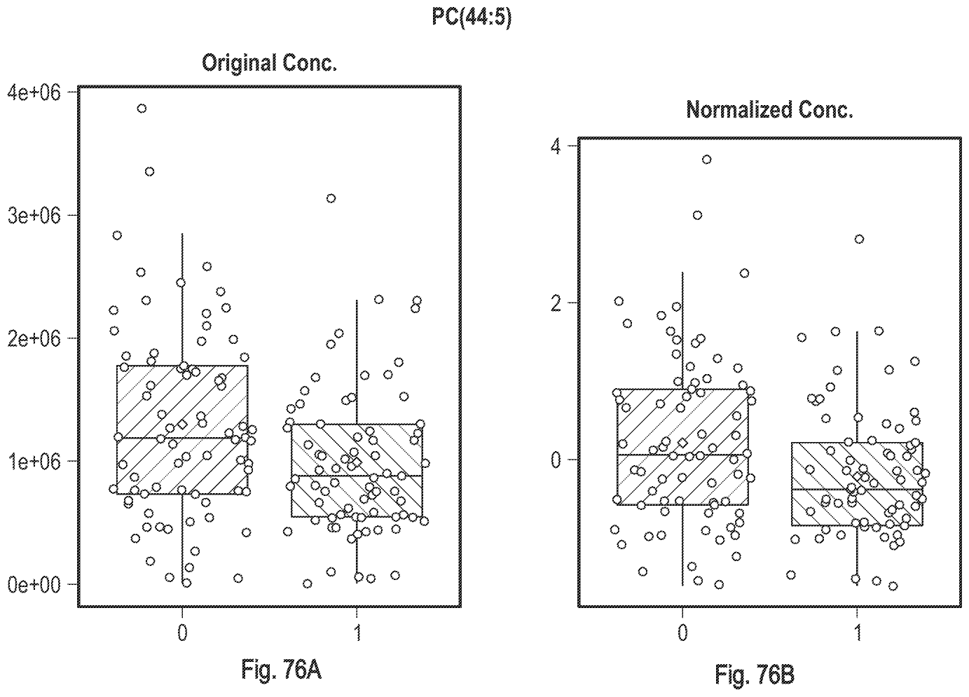

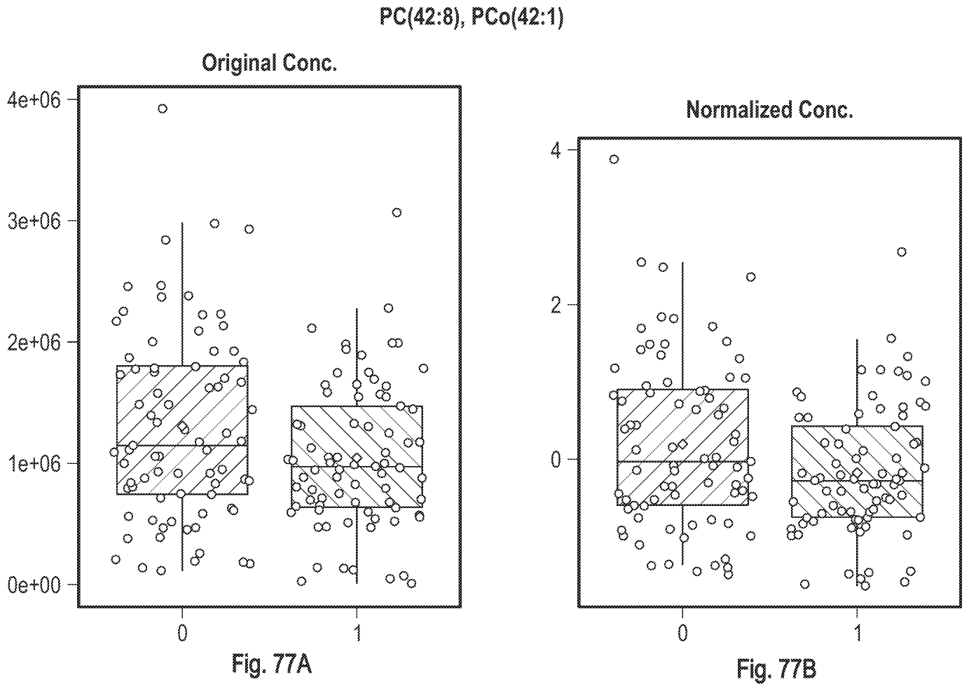

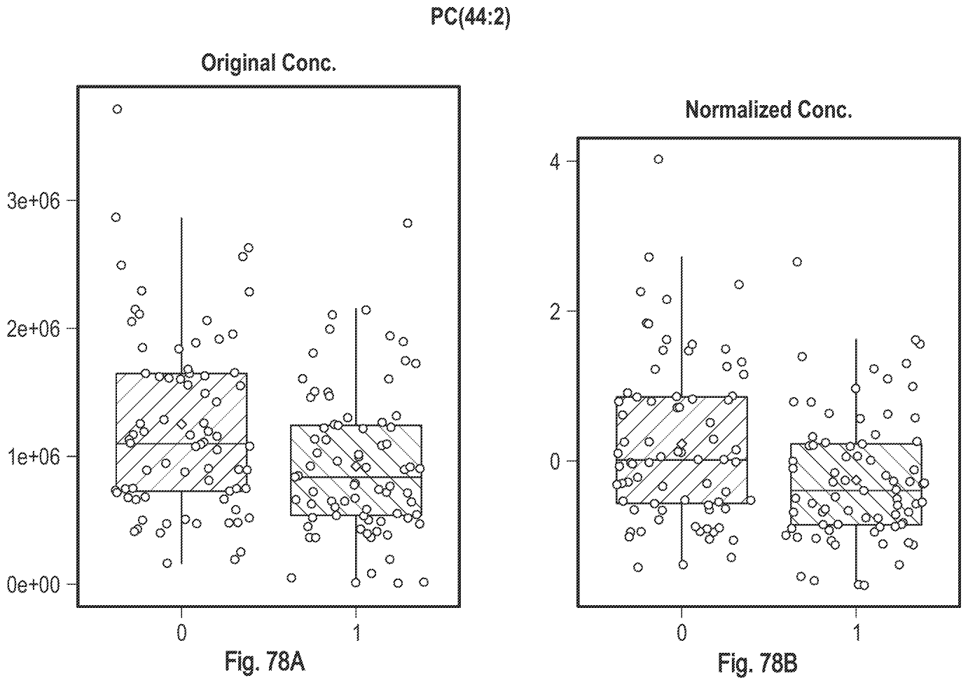

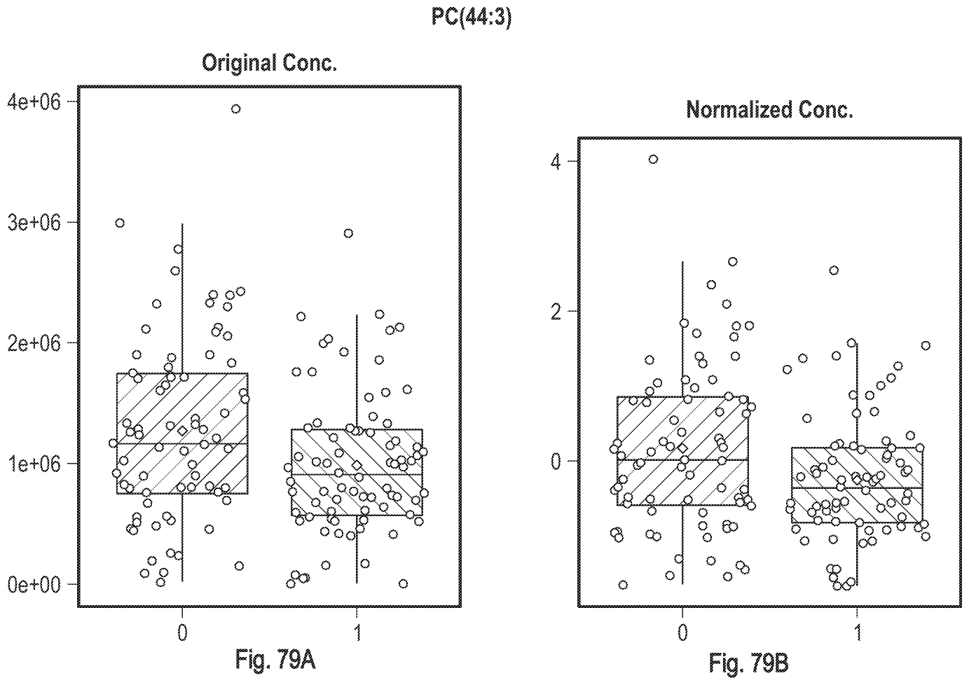

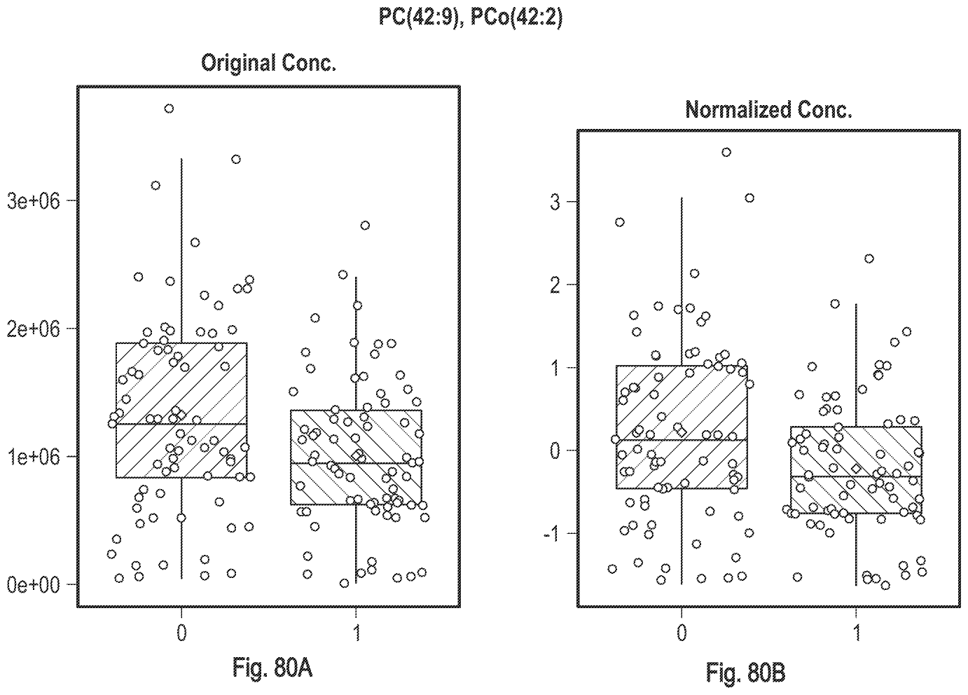

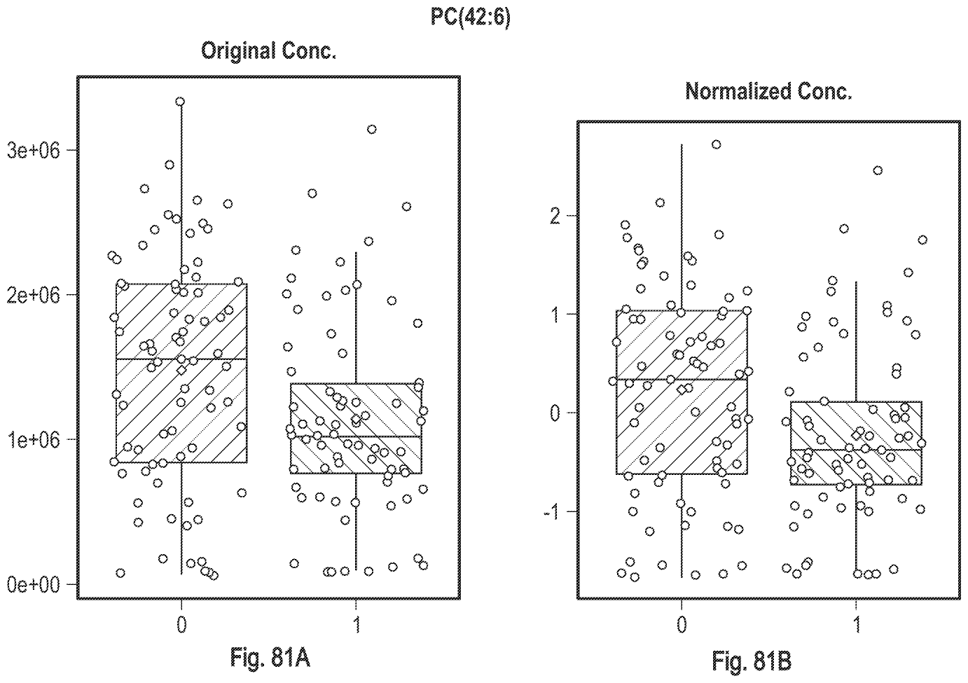

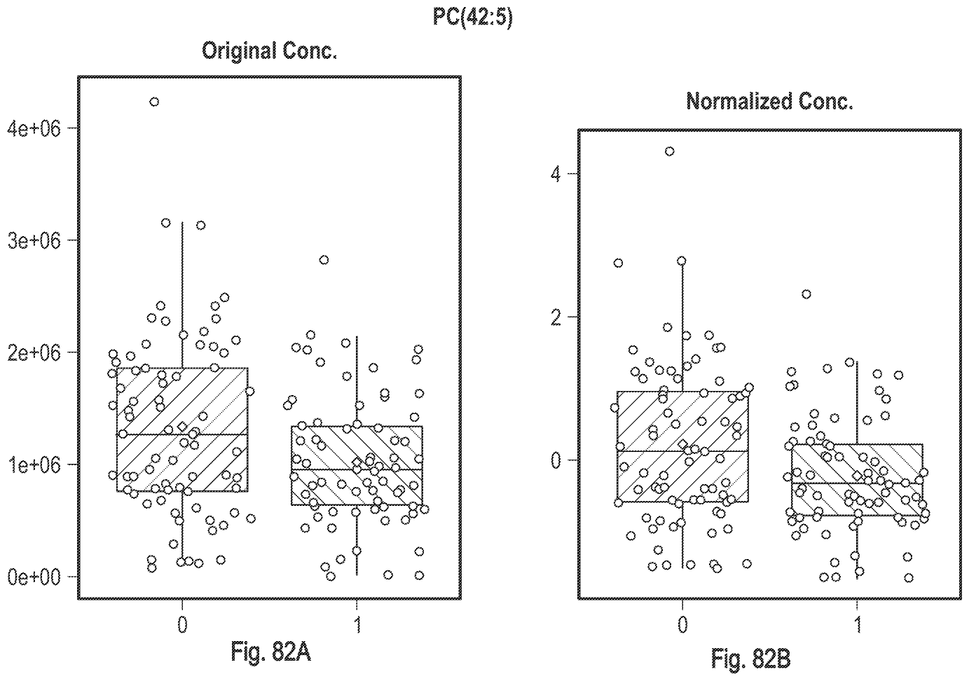

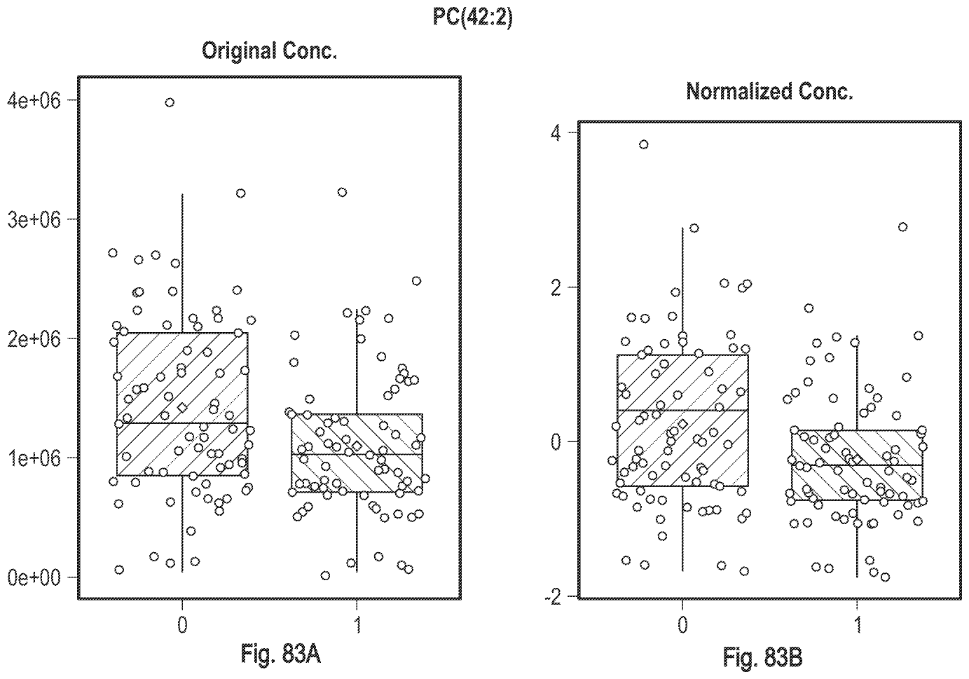

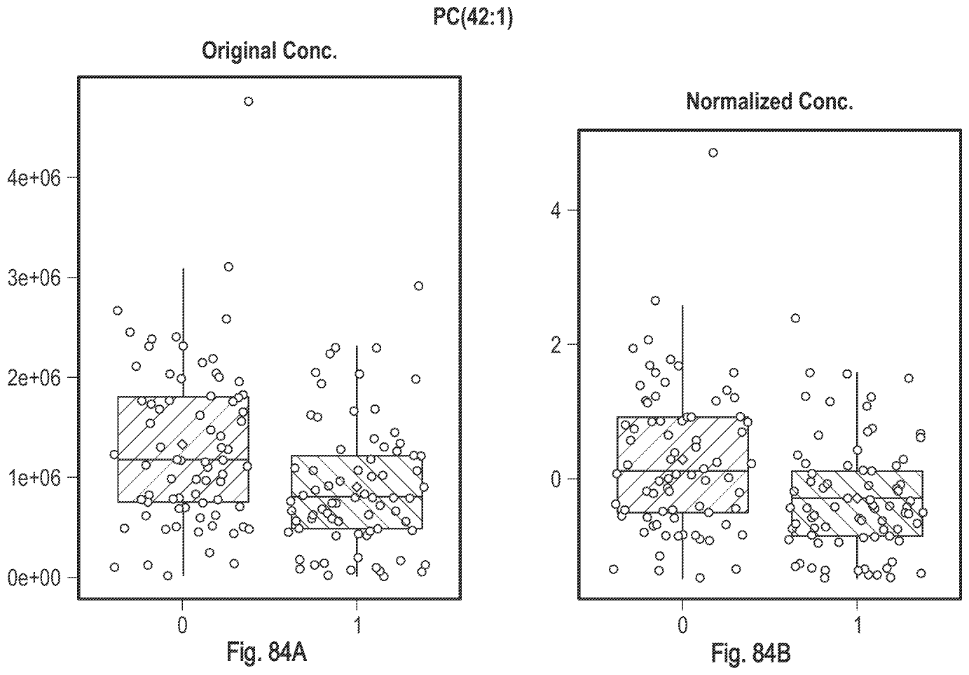

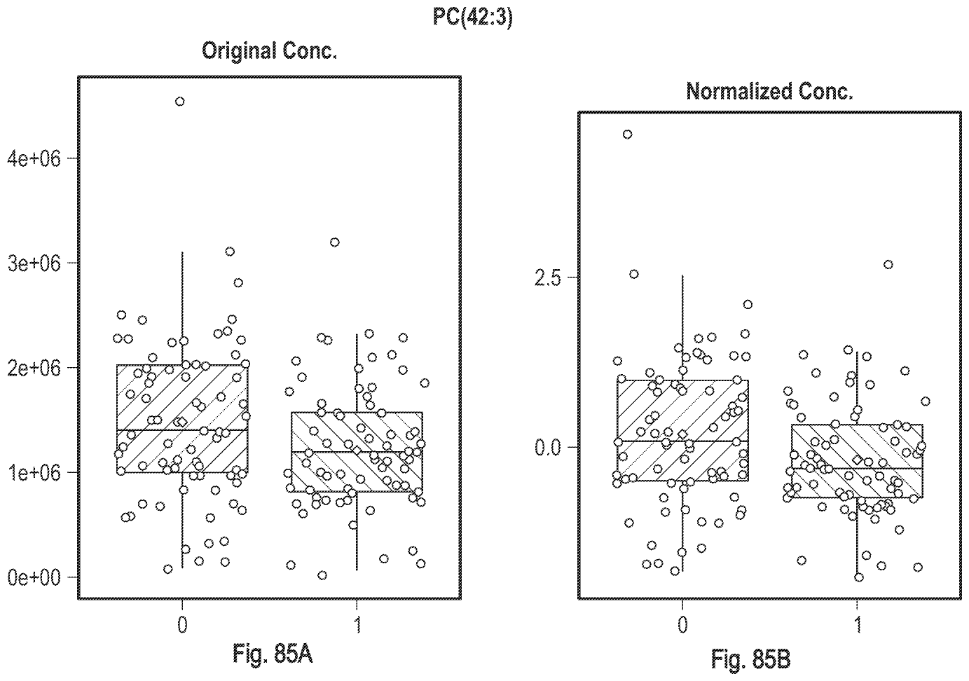

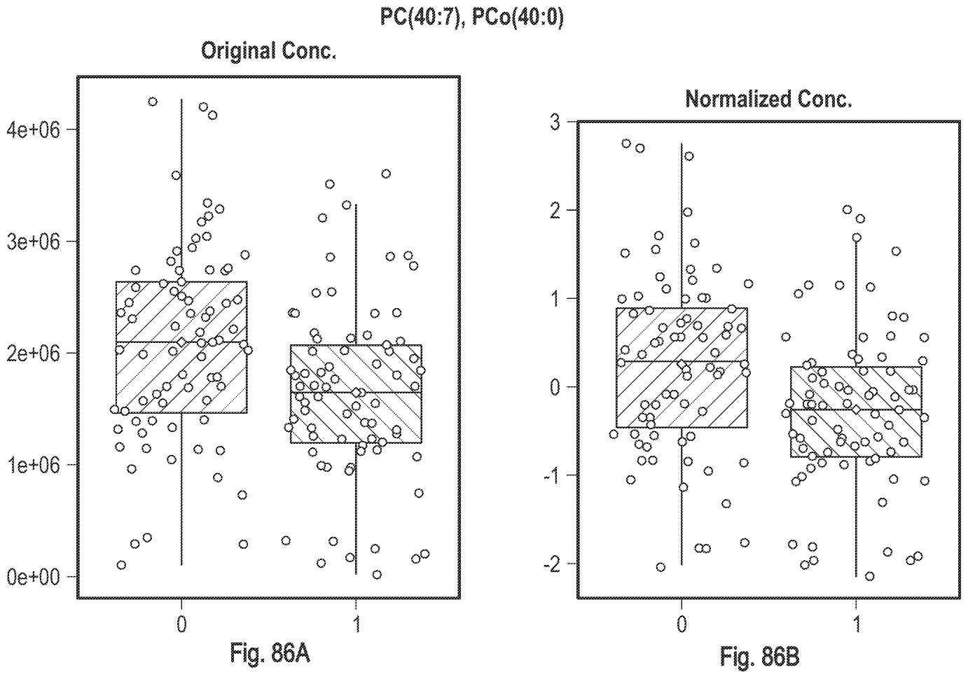

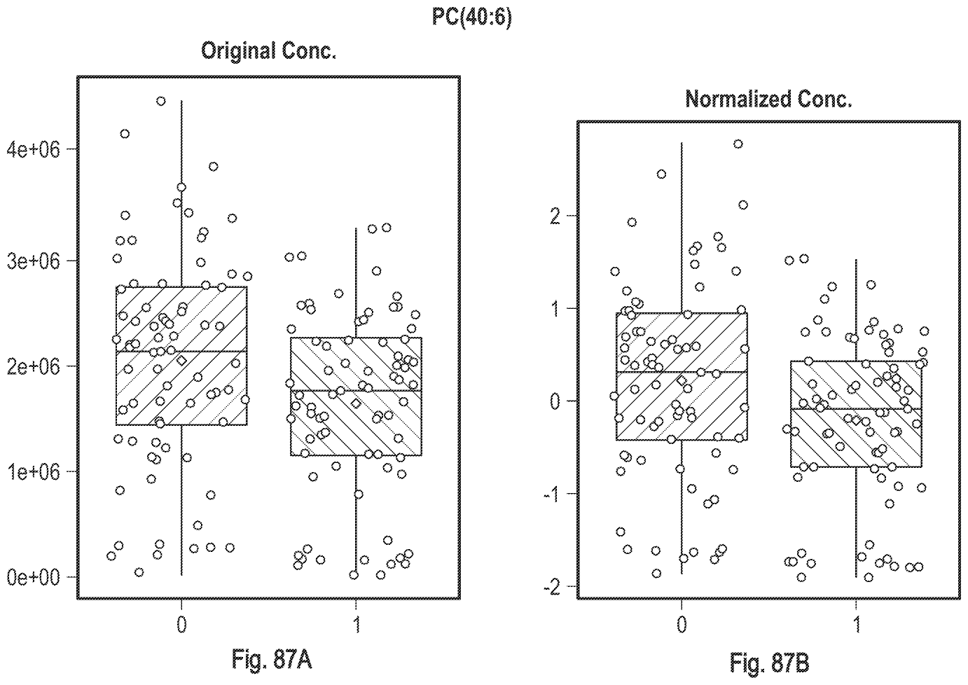

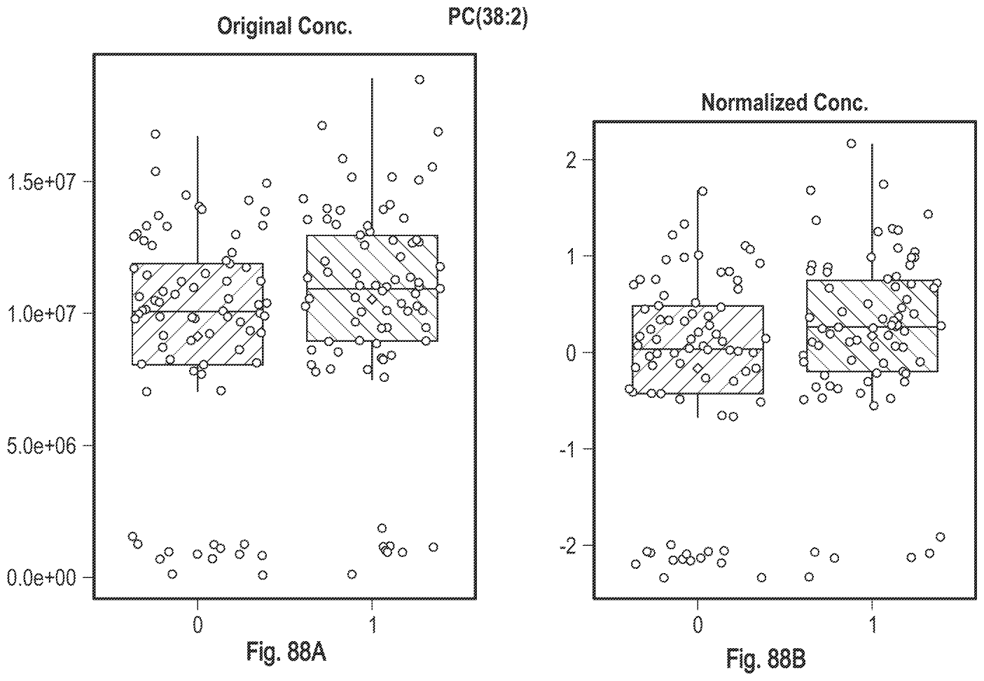

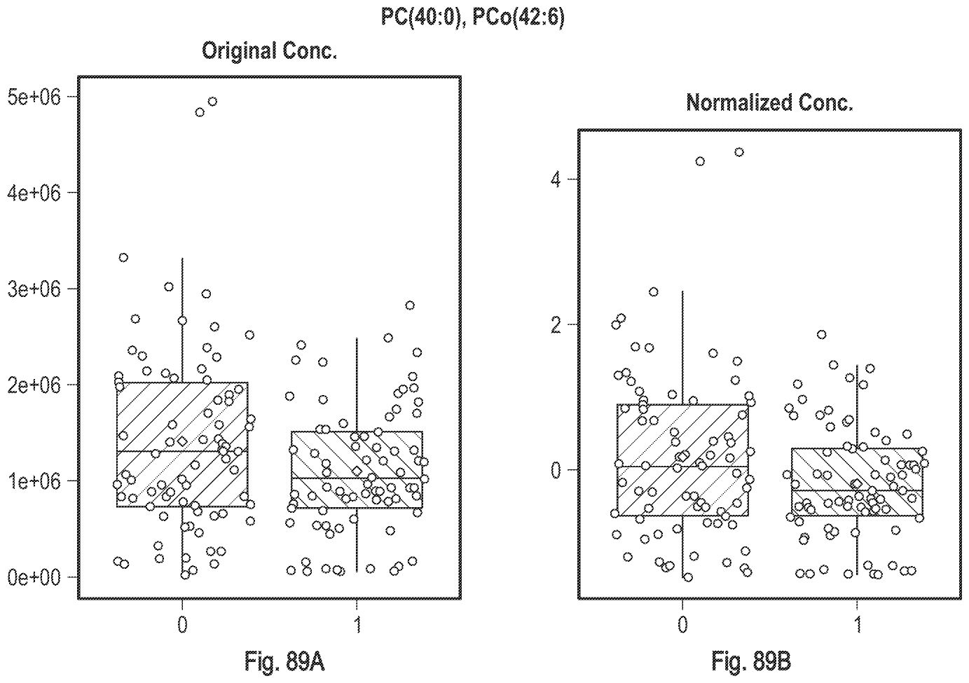

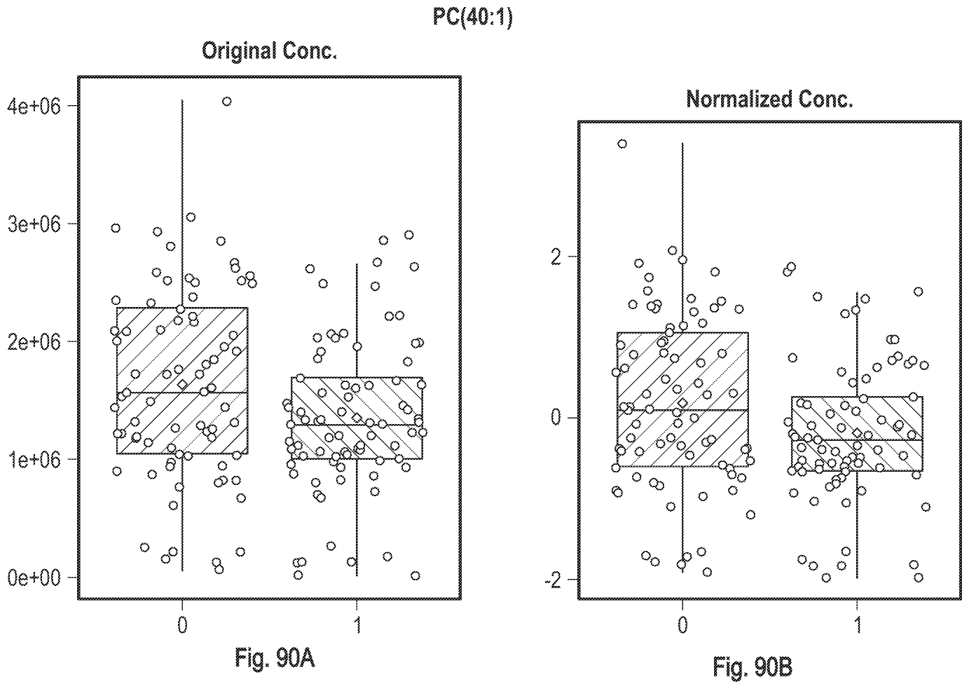

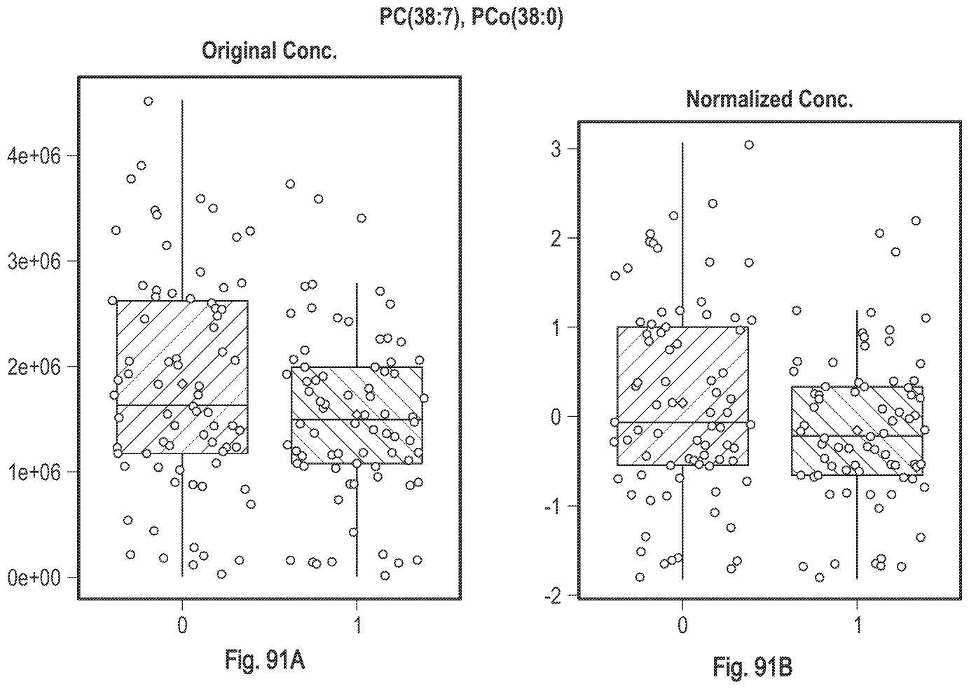

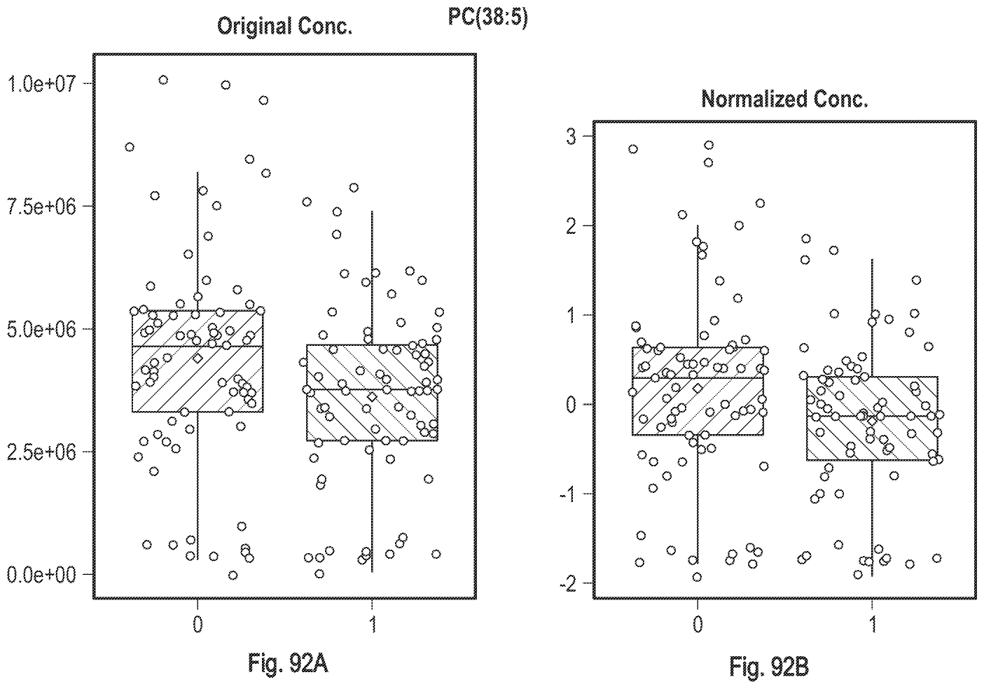

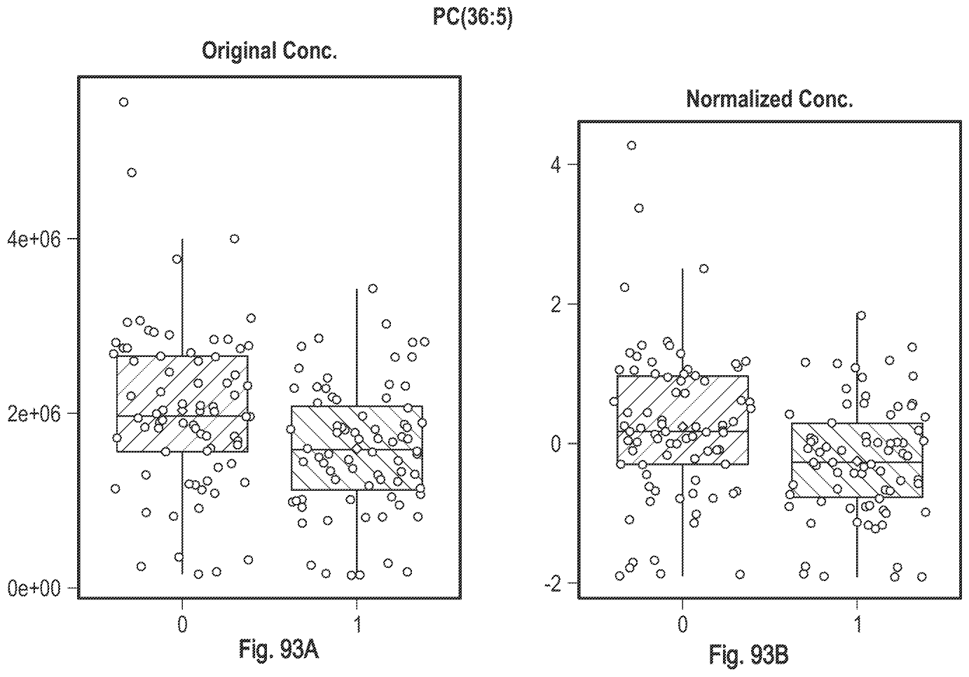

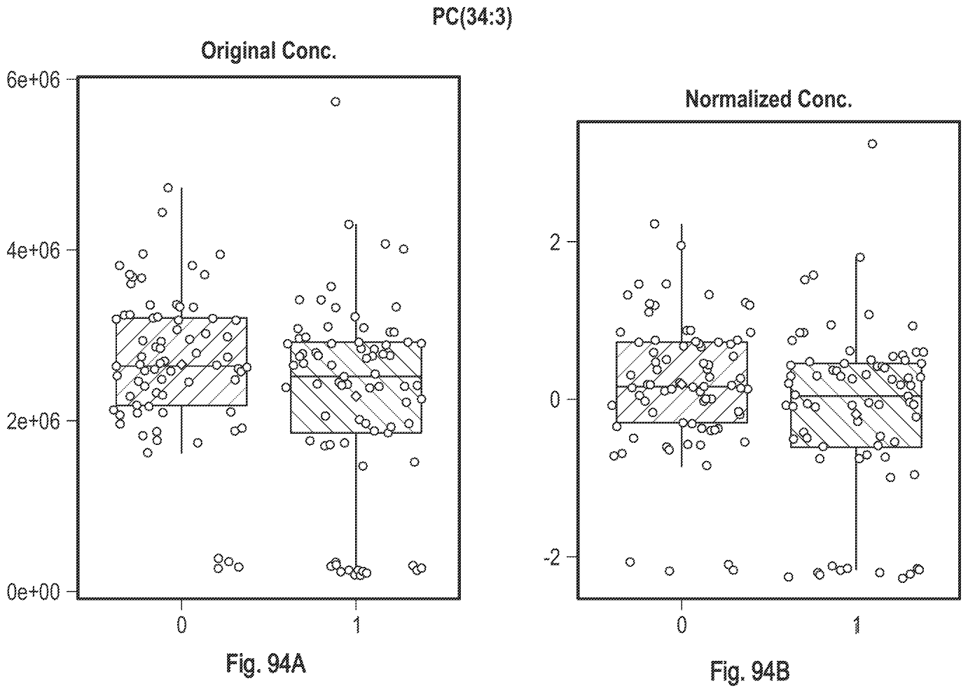

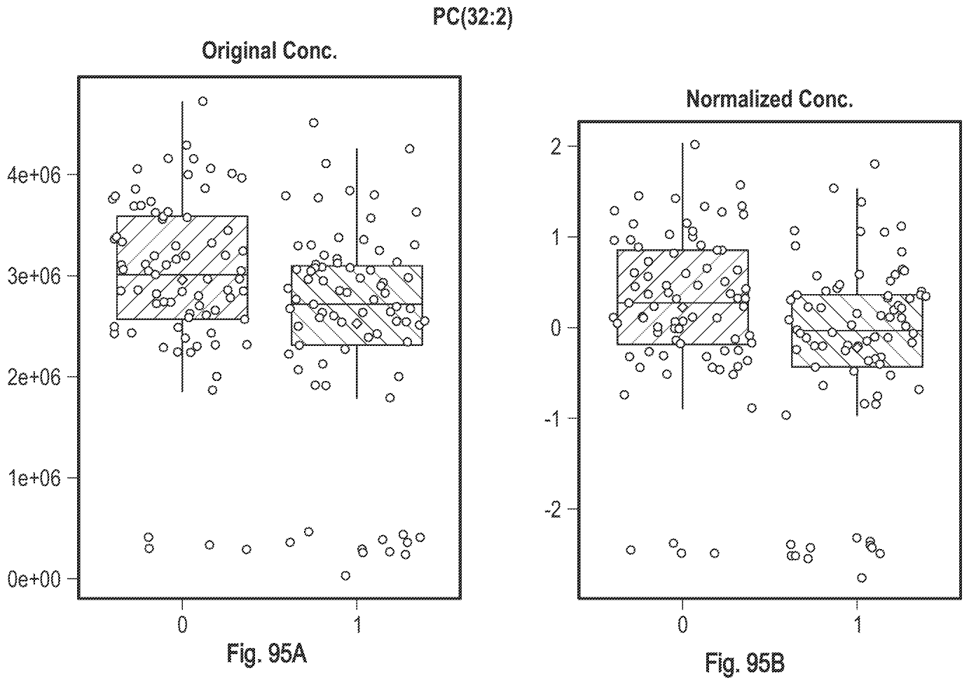

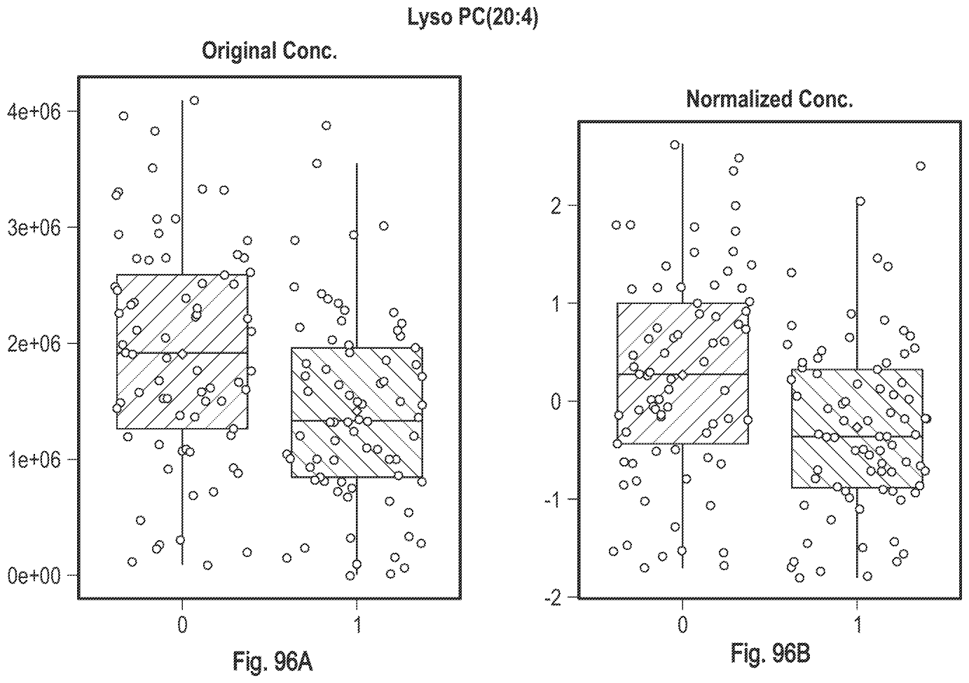

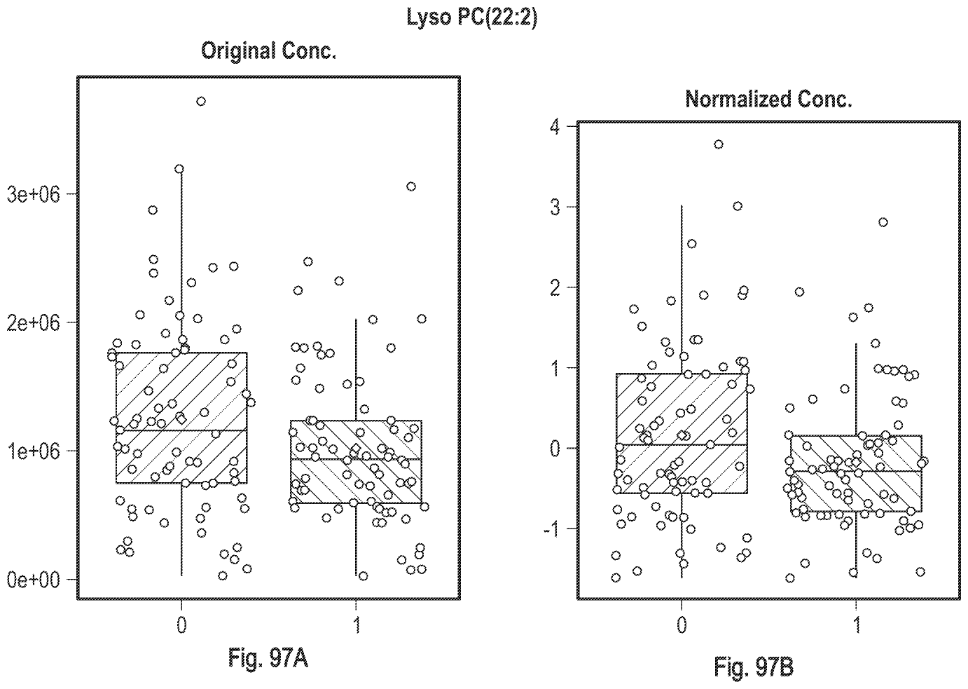

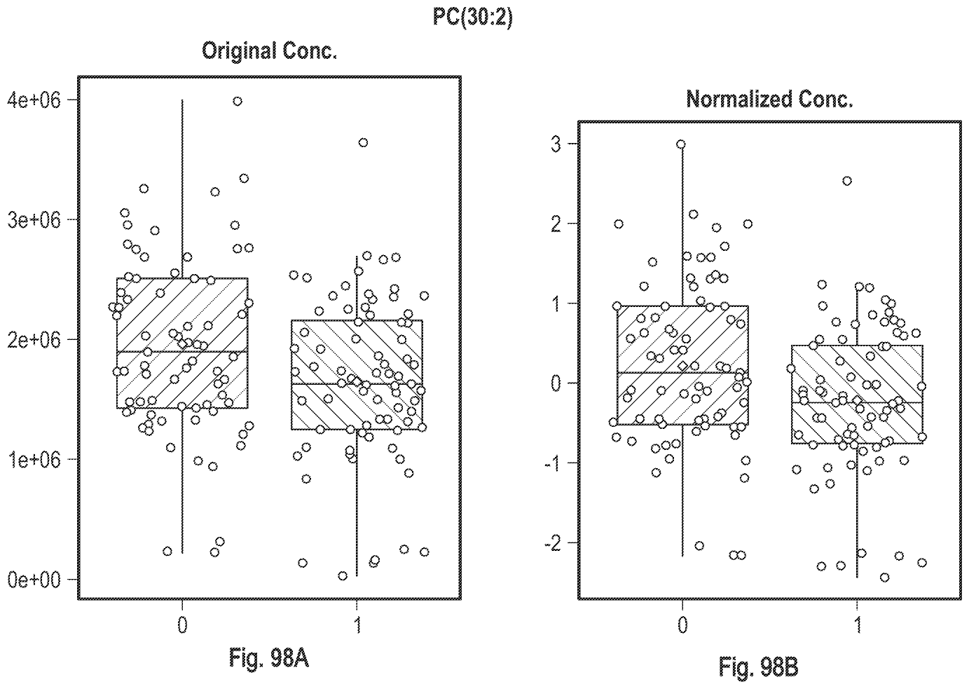

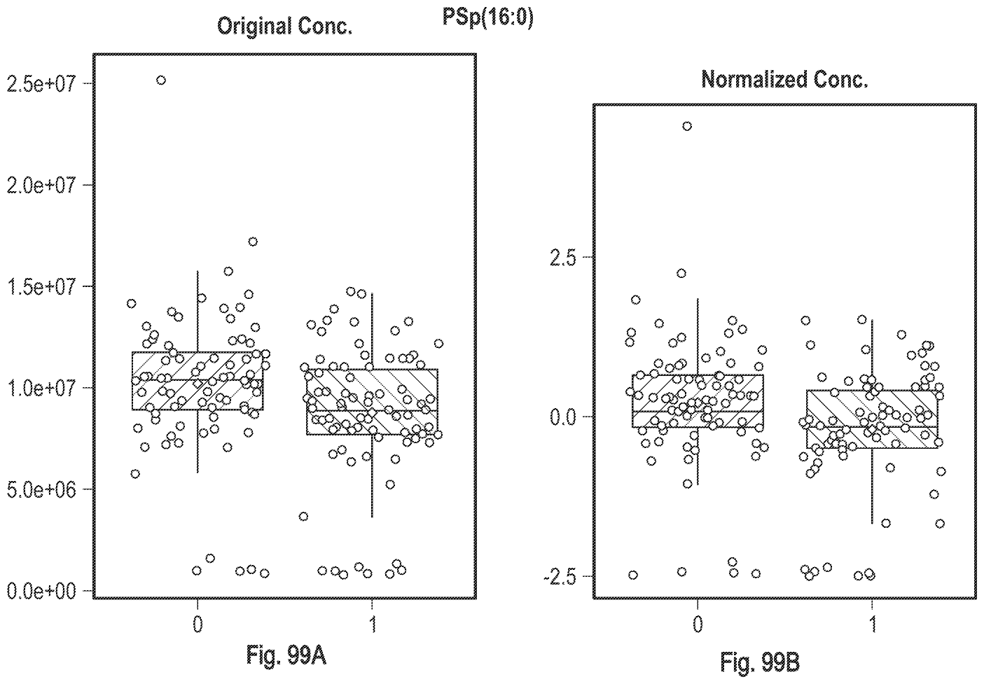

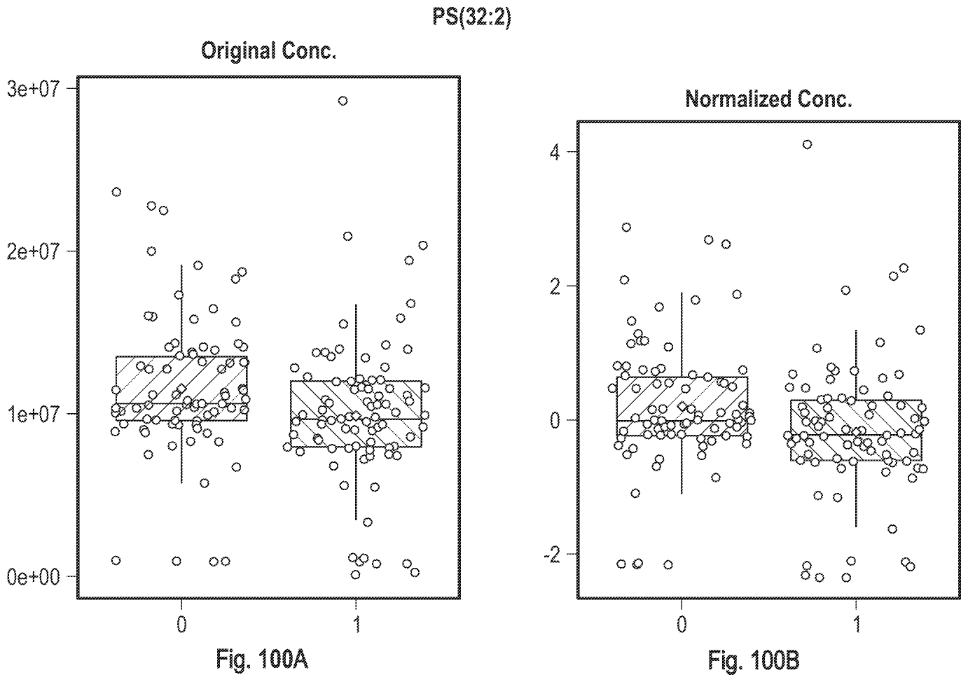

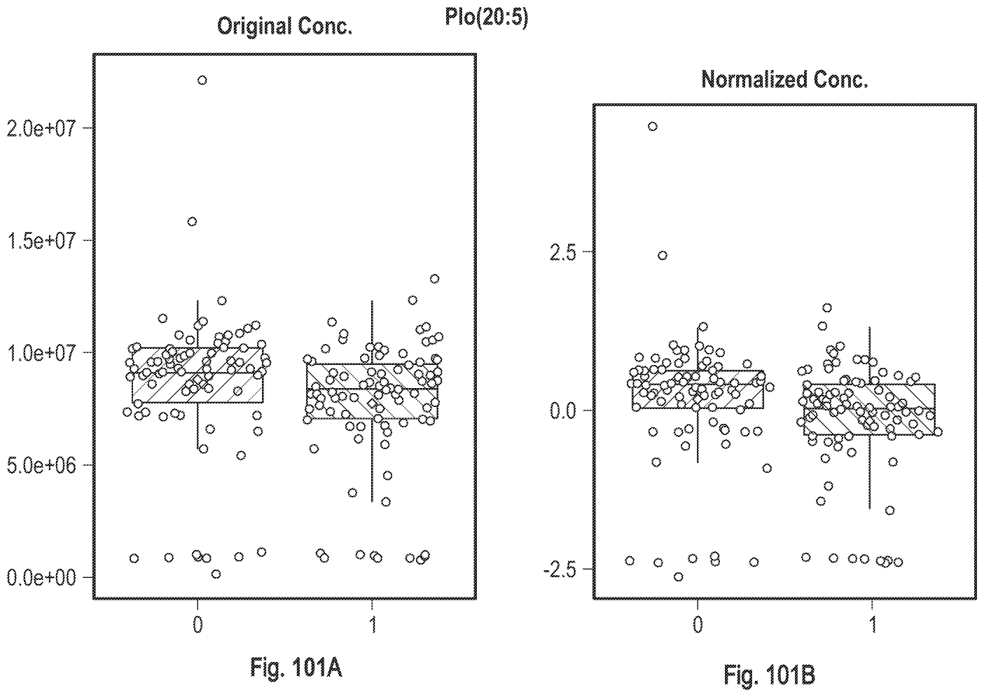

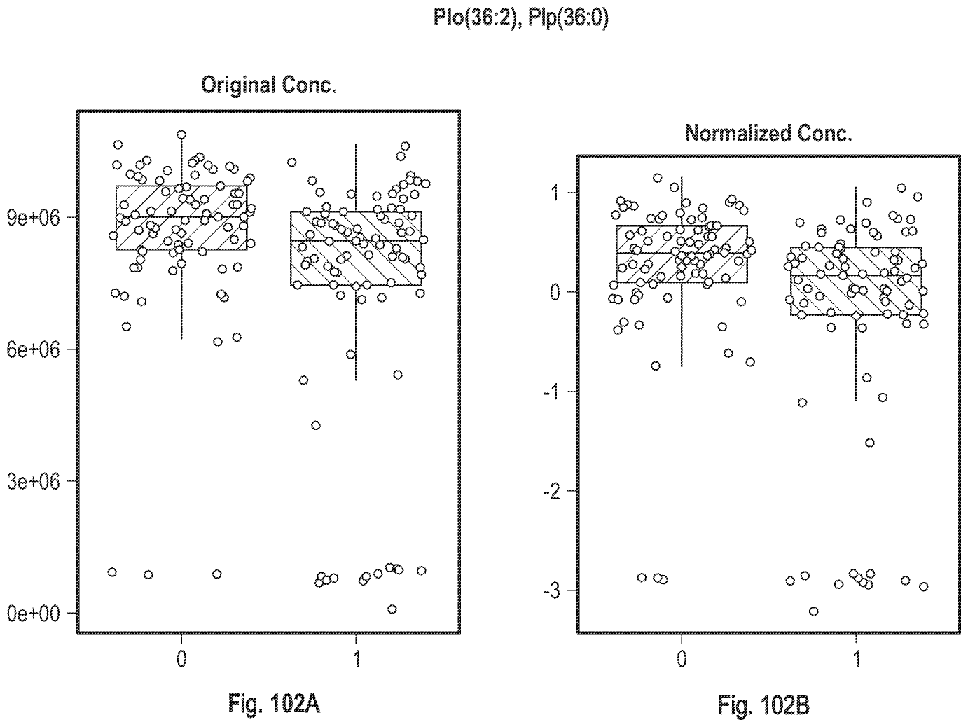

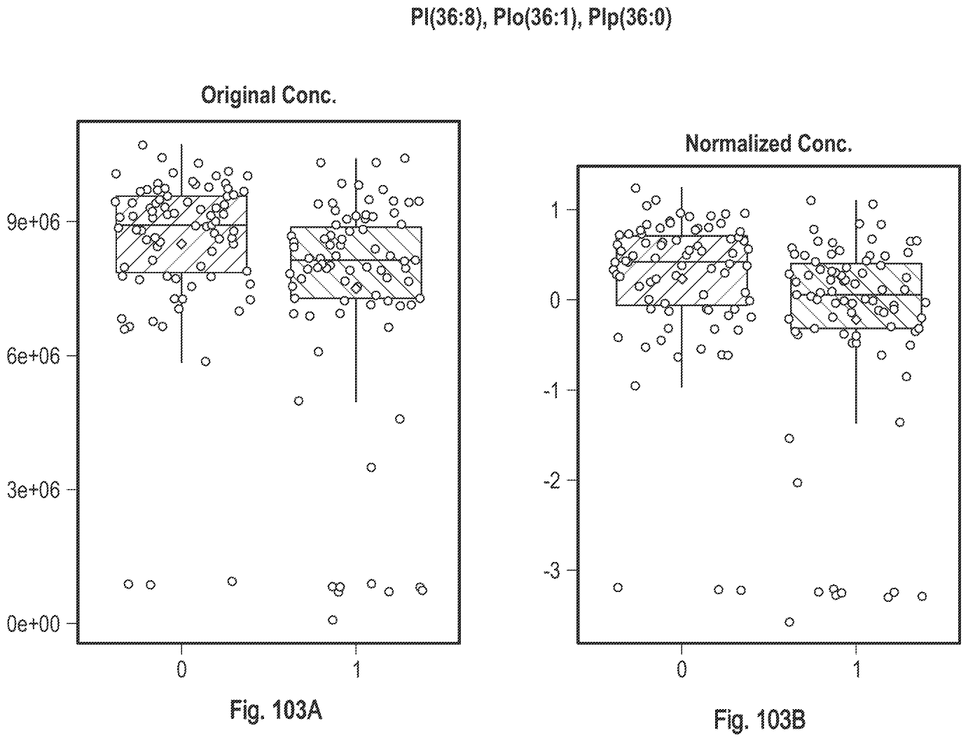

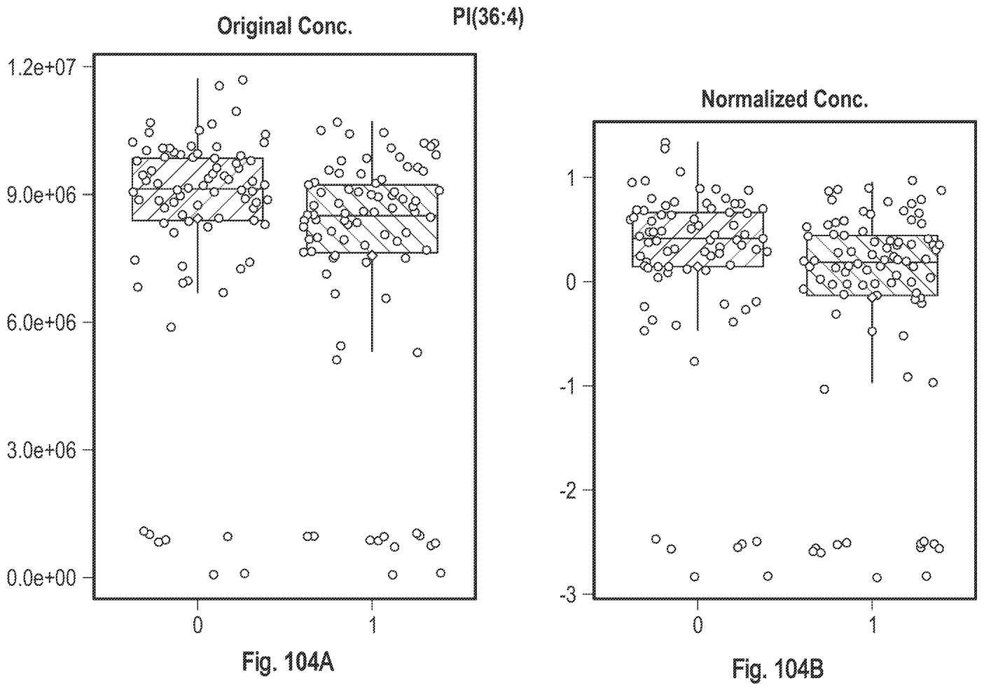

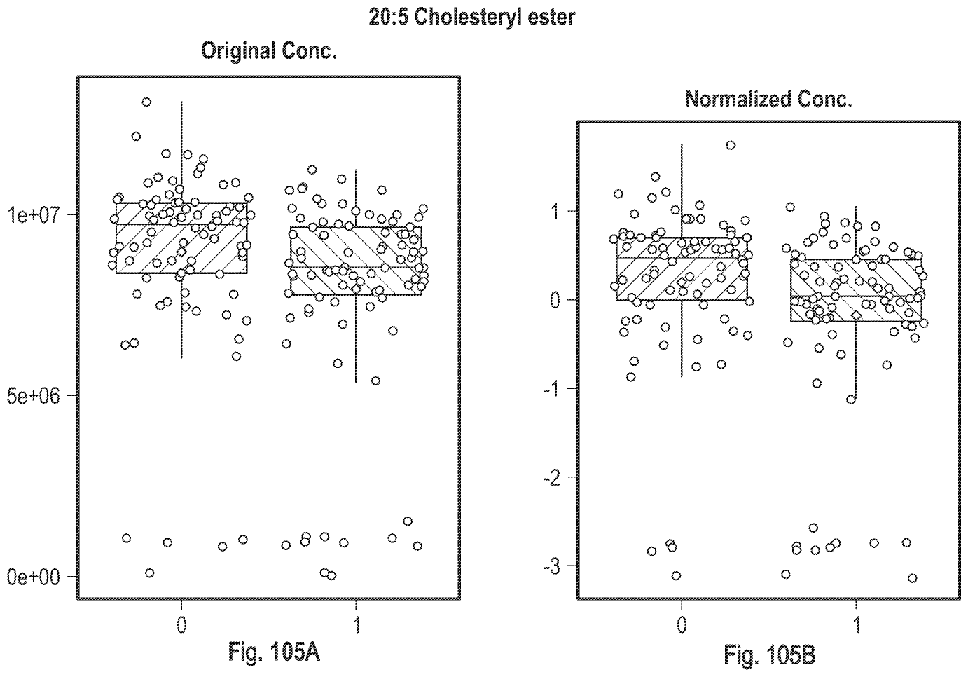

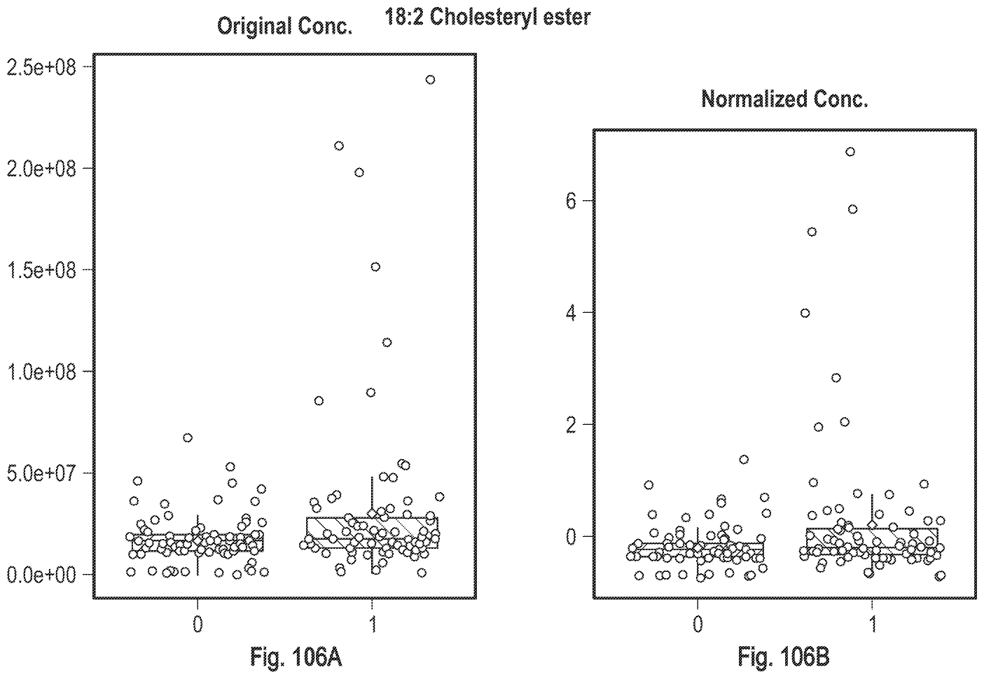

TABLE 2

| | ROC Curve | |
|---|---|---|
| Lipid(s)/Lipid Ratio | or Boxplot | Relevance to Pregnancy Status |
| First lipid/lipid ratio in group: | FIG. 1A | Able to apply these lipids/lipid ratios to accurately |
| TAG(54:2)_FA 18:1 | (AUC: 0.722) | distinguish between pregnant and non-pregnant |
| | | females |
| First lipid/lipid ratio in group: | FIG. 2A | Able to apply these lipids/lipid ratios to accurately |
| PCo(40:4)/SM(d18:0/20:0) | (AUC: 0.815) | distinguish between pregnant and non-pregnant |
| | | females |
| First lipid/lipid ratio in group: | FIG. 3A | Able to apply these lipids/lipid ratios to accurately |
| PCo(40:4)/SM(d18:0/20:0) | (AUC: 0.871) | distinguish between pregnant and non-pregnant |
| | | females |
| First lipid/lipid ratio in group: | FIG. 4A | Able to apply these lipids/lipid ratios to accurately |
| PE(42:10), PEp(42:2)/ | (AUC: 0.704) | distinguish between pregnant and non-pregnant |
| SM(d18:1/16:0) | | females |
| First lipid/lipid ratio in group: | FIG. 5A | Able to apply these lipids/lipid ratios to accurately |
| PS(36:3)/PS(38:1) | (AUC: 0.818) | distinguish between pregnant and non-pregnant |
| | | females |
| First lipid/lipid ratio in group: | FIG. 6A | Able to apply these lipids/lipid ratios to accurately |
| PCo(40:6)/SM(d18:1/16:0) | (AUC: 0.737) | distinguish between pregnant and non-pregnant |
| | | females |
| First lipid/lipid ratio in group: | FIG. 7A | Able to apply these lipids/lipid ratios to accurately |
| Plo(36:2), Plp(36:1)/PI(34:2) | (AUC: 0.784) | distinguish between pregnant and non-pregnant |
| | | females |
| First lipid/lipid ratio in group: | FIG. 8A | Able to apply these lipids/lipid ratios to accurately |
| Cer(d14:1/20:1(20H))/ | (AUC: 0.939) | distinguish between pregnant and non-pregnant |
| TAG(52:3)_FA18:1 | | females |
| First lipid/lipid ratio in group: | FIG. 9A | Able to apply these lipids/lipid ratios to accurately |
| SM(d18:1/26:0)/ | (AUC: 0.975) | distinguish between pregnant and non-pregnant |
| SM(d18:1/18:0) | | females |
| First lipid/lipid ratio in group: | FIG. 10A | Able to apply these lipids/lipid ratios to accurately |
| PSp(38:6), PS(36:0) | (AUC: 0.852) | distinguish between pregnant and non-pregnant |
| | | females |
| First lipid/lipid ratio in group: | FIG. 11A | Able to apply these lipids/lipid ratios to accurately |
| PE(38:7), PEo(38:0)/ | (AUC: 0.797) | distinguish between pregnant and non-pregnant |
| PCo(36:5) | | females |
| Cer(d14:1/24:0) (20H) | FIG. 12 | Lipid Has Significantly Higher Concentration in |
| | | Unsuccessful Pregnancy Group |
| TAG(50:2)_FA 16:0 | FIG. 13 | Lipid Has Significantly Higher Concentration in |
| | | Unsuccessful Pregnancy Group |
| Cer(d18:1/16:0) | FIG. 14 | Lipid Has Significantly Higher Concentration in |
| | | Successful Pregnancy Group |
| TAG(50:1)_FA 18:1 | FIG. 15 | Lipid Has Significantly Higher Concentration in |
| | | Unsuccessful Pregnancy Group |
| TAG(52:1)_FA 18:1 | FIG. 16 | Lipid Has Significantly Higher Concentration in |
| | | Unsuccessful Pregnancy Group |
| Lyso PC(19:2) | FIG. 17 | Lipid Has Significantly Higher Concentration in |
| | | Unsuccessful Pregnancy Group |
| Lyso PC(17:2) | FIG. 18 | Lipid Has Significantly Higher Concentration in |
| | | Unsuccessful Pregnancy Group |
| Lyso PC(17:1) | FIG. 19 | Lipid Has Significantly Higher Concentration in |
| | | Unsuccessful Pregnancy Group |
| Lyso PC(17:0) | FIG. 20 | Lipid Has Significantly Higher Concentration in |
| | | Unsuccessful Pregnancy Group |
| Lyso PC(16:1) | FIG. 21 | Lipid Has Significantly Higher Concentration in |
| | | Unsuccessful Pregnancy Group |
| SM(d18:2/20:1) | FIG. 22 | Lipid Has Significantly Higher Concentration in |
| | | Unsuccessful Pregnancy Group |
| SM(d18:0/26:0) | FIG. 23 | Lipid Has Significantly Higher Concentration in |
| | | Unsuccessful Pregnancy Group |
| SM(d16:1/24:0) | FIG. 24 | Lipid Has Significantly Higher Concentration in |
| | | Successful Pregnancy Group |
| SM(d18:1/26:0) | FIG. 25 | Lipid Has Significantly Higher Concentration in |
| | | Unsuccessful Pregnancy Group |
| SM(d18:0/14:0) | FIG. 26 | Lipid Has Significantly Higher Concentration in |
| | | Unsuccessful Pregnancy Group |
| SM(d18:1/16:0) | FIG. 27 | Lipid Has Significantly Higher Concentration in |
| | | Successful Pregnancy Group |
| PEp(36:5) | FIG. 28 | Lipid Has Significantly Higher Concentration in |
| | | Unsuccessful Pregnancy Group |
| PEo(40:4) | FIG. 29 | Lipid Has Significantly Higher Concentration in |
| | | Unsuccessful Pregnancy Group |
| PEo(38:4) | FIG. 30 | Lipid Has Significantly Higher Concentration in |
| | | Unsuccessful Pregnancy Group |
| PEp(32:1) | FIG. 31 | Lipid Has Significantly Higher Concentration in |
| | | Unsuccessful Pregnancy Group |
| PEo(40:6) | FIG. 32 | Lipid Has Significantly Higher Concentration in |
| | | Unsuccessful Pregnancy Group |
| PEo(38:6) | FIG. 33 | Lipid Has Significantly Higher Concentration in |
| | | Unsuccessful Pregnancy Group |
| PEo(36:5) | FIG. 34 | Lipid Has Significantly Higher Concentration in |
| | | Unsuccessful Pregnancy Group |
| PEo(32:5) | FIG. 35 | Lipid Has Significantly Higher Concentration in |
| | | Unsuccessful Pregnancy Group |
| PEo(34:0) | FIG. 36 | Lipid Has Significantly Higher Concentration in |
| | | Unsuccessful Pregnancy Group |
| PEo(34:3) | FIG. 37 | Lipid Has Significantly Higher Concentration in |
| | | Unsuccessful Pregnancy Group |
| PEo(32:1) | FIG. 38 | Lipid Has Significantly Higher Concentration in |
| | | Unsuccessful Pregnancy Group |
| PEo(28:0) | FIG. 39 | Lipid Has Significantly Higher Concentration in |
| | | Unsuccessful Pregnancy Group |
| PE(42:10), PEp (42:2) | FIG. 40 | Lipid Has Significantly Higher Concentration in |
| | | Unsuccessful Pregnancy Group |
| PE(42:4) | FIG. 41 | Lipid Has Significantly Higher Concentration in |
| | | Unsuccessful Pregnancy Group |
| PE(40:9), PEo(40:2) | FIG. 42 | Lipid Has Significantly Higher Concentration in |
| | | Unsuccessful Pregnancy Group |
| PE(42:6) | FIG. 43 | Lipid Has Significantly Higher Concentration in |
| | | Unsuccessful Pregnancy Group |
| PE(42:2) | FIG. 44 | Lipid Has Significantly Higher Concentration in |
| | | Unsuccessful Pregnancy Group |
| PE(42:3) | FIG. 45 | Lipid Has Significantly Higher Concentration in |
| | | Unsuccessful Pregnancy Group |
| PE(40:8), PEo(40:1) | FIG. 46 | Lipid Has Significantly Higher Concentration in |
| | | Unsuccessful Pregnancy Group |
| PE(38:8), PEo(38:1) | FIG. 47 | Lipid Has Significantly Higher Concentration in |
| | | Unsuccessful Pregnancy Group |
| PE(40:7), PEo(40:0) | FIG. 48 | Lipid Has Significantly Higher Concentration in |
| | | Unsuccessful Pregnancy Group |
| PE(38:7), PEo(38:0) | FIG. 49 | Lipid Has Significantly Higher Concentration in |
| | | Unsuccessful Pregnancy Group |
| PE(40:1) | FIG. 50 | Lipid Has Significantly Higher Concentration in |
| | | Unsuccessful Pregnancy Group |
| PE(40:6) | FIG. 51 | Lipid Has Significantly Higher Concentration in |
| | | Unsuccessful Pregnancy Group |
| PE(38:0), PE(40:6) | FIG. 52 | Lipid Has Significantly Higher Concentration in |
| | | Unsuccessful Pregnancy Group |
| PE(34:3) | FIG. 53 | Lipid Has Significantly Higher Concentration in |
| | | Unsuccessful Pregnancy Group |
| PE(36:4) | FIG. 54 | Lipid Has Significantly Higher Concentration in |
| | | Unsuccessful Pregnancy Group |
| PE(36:7), PEo(36:0) | FIG. 55 | Lipid Has Significantly Higher Concentration in |
| | | Unsuccessful Pregnancy Group |
| PE(34:4) | FIG. 56 | Lipid Has Significantly Higher Concentration in |
| | | Unsuccessful Pregnancy Group |
| PE(36:1) | FIG. 57 | Lipid Has Significantly Higher Concentration in |
| | | Successful Pregnancy Group |
| PE(32:2) | FIG. 58 | Lipid Has Significantly Higher Concentration in |
| | | Unsuccessful Pregnancy Group |
| PE(18:0), Lyso PE(18:0) | FIG. 59 | Lipid Has Significantly Higher Concentration in |
| | | Unsuccessful Pregnancy Group |
| PE(32:0) | FIG. 60 | Lipid Has Significantly Higher Concentration in |
| | | Unsuccessful Pregnancy Group |
| PE(32:1) | FIG. 61 | Lipid Has Significantly Higher Concentration in |
| | | Unsuccessful Pregnancy Group |
| PE(22:2), Lyso PE(22:2) | FIG. 62 | Lipid Has Significantly Higher Concentration in |
| | | Unsuccessful Pregnancy Group |
| PE(30:1) | FIG. 63 | Lipid Has Significantly Higher Concentration in |
| | | Unsuccessful Pregnancy Group |
| PCp(42:4) | FIG. 64 | Lipid Has Significantly Higher Concentration in |
| | | Unsuccessful Pregnancy Group |
| PCo(42:6) | FIG. 65 | Lipid Has Significantly Higher Concentration in |
| | | Unsuccessful Pregnancy Group |
| PCo(40:6) | FIG. 66 | Lipid Has Significantly Higher Concentration in |
| | | Unsuccessful Pregnancy Group |
| PCo(40:4) | FIG. 67 | Lipid Has Significantly Higher Concentration in |
| | | Unsuccessful Pregnancy Group |
| PCo(40:5) | FIG. 68 | Lipid Has Significantly Higher Concentration in |
| | | Unsuccessful Pregnancy Group |
| PCo(38:4) | FIG. 69 | Lipid Has Significantly Higher Concentration in |
| | | Unsuccessful Pregnancy Group |
| PCo(38:3) | FIG. 70 | Lipid Has Significantly Higher Concentration in |
| | | Unsuccessful Pregnancy Group |
| PCo(32:3) | FIG. 71 | Lipid Has Significantly Higher Concentration in |
| | | Unsuccessful Pregnancy Group |
| PCo(32:0) | FIG. 72 | Lipid Has Significantly Higher Concentration in |
| | | Successful Pregnancy Group |
| PC(40:10), PCo(40:3) | FIG. 73 | Lipid Has Significantly Higher Concentration in |
| | | Unsuccessful Pregnancy Group |
| PC(42:10), PCo(42:3) | FIG. 74 | Lipid Has Significantly Higher Concentration in |
| | | Unsuccessful Pregnancy Group |
| PC(44:10), PCo(44:3) | FIG. 75 | Lipid Has Significantly Higher Concentration in |
| | | Unsuccessful Pregnancy Group |
| PC(44:5) | FIG. 76 | Lipid Has Significantly Higher Concentration in |
| | | Unsuccessful Pregnancy Group |
| PC(42:8), PCo(42:1) | FIG. 77 | Lipid Has Significantly Higher Concentration in |
| | | Unsuccessful Pregnancy Group |
| PC(44:2) | FIG. 78 | Lipid Has Significantly Higher Concentration in |
| | | Unsuccessful Pregnancy Group |
| PC(44:3) | FIG. 79 | Lipid Has Significantly Higher Concentration in |
| | | Unsuccessful Pregnancy Group |
| PC(42:9), PCo(42:2) | FIG. 80 | Lipid Has Significantly Higher Concentration in |
| | | Unsuccessful Pregnancy Group |
| PC(42:6) | FIG. 81 | Lipid Has Significantly Higher Concentration in |
| | | Unsuccessful Pregnancy Group |
| PC(42:5) | FIG. 82 | Lipid Has Significantly Higher Concentration in |
| | | Unsuccessful Pregnancy Group |
| PC(42:2) | FIG. 83 | Lipid Has Significantly Higher Concentration in |
| | | Unsuccessful Pregnancy Group |
| PC(42:1) | FIG. 84 | Lipid Has Significantly Higher Concentration in |
| | | Unsuccessful Pregnancy Group |
| PC(42:3) | FIG. 85 | Lipid Has Significantly Higher Concentration in |
| | | Unsuccessful Pregnancy Group |
| PC(40:7), PCo(40:0) | FIG. 86 | Lipid Has Significantly Higher Concentration in |
| | | Unsuccessful Pregnancy Group |
| PC(40:6) | FIG. 87 | Lipid Has Significantly Higher Concentration in |
| | | Unsuccessful Pregnancy Group |
| PC(38:2) | FIG. 88 | Lipid Has Significantly Higher Concentration in |
| | | Successful Pregnancy Group |
| PC(40:0), PCo(42:6) | FIG. 89 | Lipid Has Significantly Higher Concentration in |
| | | Unsuccessful Pregnancy Group |
| PC(40:1) | FIG. 90 | Lipid Has Significantly Higher Concentration in |
| | | Unsuccessful Pregnancy Group |
| PC(38:7), PCo(38:0) | FIG. 91 | Lipid Has Significantly Higher Concentration in |
| | | Unsuccessful Pregnancy Group |
| PC(38:5) | FIG. 92 | Lipid Has Significantly Higher Concentration in |
| | | Unsuccessful Pregnancy Group |
| PC(36:5) | FIG. 93 | Lipid Has Significantly Higher Concentration in |
| | | Unsuccessful Pregnancy Group |
| PC(34:3) | FIG. 94 | Lipid Has Significantly Higher Concentration in |
| | | Unsuccessful Pregnancy Group |
| PC(32:2) | FIG. 95 | Lipid Has Significantly Higher Concentration in |
| | | Unsuccessful Pregnancy Group |
| Lyso PC(20:4) | FIG. 96 | Lipid Has Significantly Higher Concentration in |
| | | Unsuccessful Pregnancy Group |
| Lyso PC(22:2) | FIG. 97 | Lipid Has Significantly Higher Concentration in |
| | | Unsuccessful Pregnancy Group |
| PC(30:2) | FIG. 98 | Lipid Has Significantly Higher Concentration in |
| | | Unsuccessful Pregnancy Group |
| PSp(16:0) | FIG. 99 | Lipid Has Significantly Higher Concentration in |
| | | Unsuccessful Pregnancy Group |
| PS(32:2) | FIG. 100 | Lipid Has Significantly Higher Concentration in |
| | | Unsuccessful Pregnancy Group |
| Plo(20:5) | FIG. 101 | Lipid Has Significantly Higher Concentration in |
| | | Unsuccessful Pregnancy Group |
| Plo(36:2), Plp(36:1) | FIG. 102 | Lipid Has Significantly Higher Concentration in |
| | | Unsuccessful Pregnancy Group |
| PI(36:8), Plo(36:1), Plp(36:0) | FIG. 103 | Lipid Has Significantly Higher Concentration in |
| | | Unsuccessful Pregnancy Group |
| PI(36:4) | FIG. 104 | Lipid Has Significantly Higher Concentration in |
| | | Unsuccessful Pregnancy Group |
| 20:5 Cholesteryl Ester | FIG. 105 | Lipid Has Significantly Higher Concentration in |
| | | Unsuccessful Pregnancy Group |
| 18:2 Cholesteryl Ester | FIG. 106 | Lipid Has Significantly Higher Concentration in |
| | | Successful Pregnancy Group |
| 20:1 Cholesteryl Ester | FIG. 107 | Lipid Has Significantly Higher Concentration in |
| | | Successful Pregnancy Group |
| TAG(50:4)_FA 16:1 | FIG. 108 | Lipid Has Significantly Higher Concentration in |
| | | Successful Pregnancy Group |
| TAG(54:4)_FA 18:2 | FIG. 109 | Lipid Has Significantly Higher Concentration in |
| | | Unsuccessful Pregnancy Group |
| TAG(52:3)_FA 16:0 | FIG. 110 | Lipid Has Significantly Higher Concentration in |
| | | Successful Pregnancy Group |
| PC(32:1) | FIG. 111 | Lipid Has Significantly Higher Concentration in |
| | | Unsuccessful Pregnancy Group |
| PCo(34:1) | FIG. 112 | Lipid Has Significantly Higher Concentration in |
| | | Unsuccessful Pregnancy Group |

The “scope” of the present disclosure is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

Where a term is provided in the singular, other embodiments described by the plural of that term are also provided. As used herein, the terms “include,” “includes,” and “including” are to be construed as at least having the features to which they refer while not excluding any additional unspecified features. It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “configured” describes structure capable of performing a task or adopting a particular configuration. The term “configured” can be used interchangeably with other similar phrases, such as constructed, arranged, adapted, manufactured, and the like. Terms characterizing sequential order, a position, and/or an orientation are not limiting and are only referenced according to the views presented.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

What is claimed is:

1. A method for predicting in a candidate bovine surrogate dam an ability to establish a successful embryo implantation and pregnancy following embryo transfer, the method comprising: (a) obtaining vaginal fluids from a bovine surrogate dam, wherein the vaginal fluids comprises a vaginal lipidome; and (b) performing lipidome analysis on the vaginal fluids to obtain a vaginal lipid profile comprising vaginal lipid ratios, wherein an increase or decreased vaginal lipid ratio and/or individual lipid concentration in the candidate bovine surrogate dam animal is predictive of a candidate bovine surrogate dam possessing an environment that is suitable for achieving a successful embryo implantation and pregnancy or that is unsuitable for achieving a successful embryo implantation and pregnancy.

2. The method of claim 1 wherein the vaginal lipid profile comprises a vaginal lipid profile comprising a vaginal lipid ratio for a first vaginal lipid and a second vaginal lipid that is predictive of a candidate bovine surrogate dam possessing an environment that is suitable or unsuitable for achieving a successful embryo implantation and pregnancy.

3. The method of claim 2 wherein the first vaginal lipid is a sphingolipid.

4. The method of claim 3 wherein the sphingolipid is a sphingomyelin.

5. The method of claim 4 wherein the sphingomyelin is selected from the group consisting of SM(d18:1/26:0) and SM(d18:1/18:0).

6. The method of claim 2 wherein the second vaginal lipid is selected from the group consisting of a Hosphatidylcholine, Lysophosphatidylcholine, Lysophosphatidylethanolamine, Choline plasmalogen, Phosphatidylethanolamines, Ethanolamine plasmalogen, Phosphatidylethanolamine (PE)-plasmalogen, Sphingomyelin, Ceramides, Triacylglycerol, Phosphatidylserine, Phosphatidylinositol, Free fatty acids and phosphatidylglycerol, Plasmanylphosphatidylcholine, Plasmenylphosphatidylcholine, Cholesteryl ester, Plasmenylserine, and a Plasmanylserine.

7. The method of claim 6 wherein the second vaginal lipid is a phosphatidylcholine.

8. The method of claim 7 wherein the phosphatidylethanolamine is selected from the group consisting of SM(d18:1/26:0)/SM(d18:1/18:0), SM(d18:0/24:0)/SM(d18:1/18:0), PCo (32:2)/PC(38:5), SM(d16:1/18:1)/SM(d18:1/18:0), PC(38:5)/PC(30:0), PC(40:5)/SM(d18:1/18.0), PCo(32:2)/PC(40.5), SM(d18:1/18:0)/PC(38:2), PC(38:5)/SM(d18:1/18:0), PC(34:5)/PE(34.4), PCo(32.2)/PC(38.4), PCo(32:2)/PC(38.4), SM(d18:1/18:0)/PC(38.5), and PC(40.3)/SM(d18:1/18:0).

9. The method of claim 1 wherein the vaginal lipid profile comprises comprises multiple vaginal lipid ratios for at least two vaginal lipid ratios.

10. The method of claim 1 wherein the vaginal lipid profile comprises a vaginal lipid profile comprising a vaginal lipid level that is predictive of a candidate bovine surrogate dam possessing an environment that is suitable for achieving a successful embryo implantation and pregnancy.

11. The method of claim 10 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

12. The method of claim 1 wherein the vaginal lipid profile comprises a vaginal lipid profile comprising a vaginal lipid level that is predictive of a candidate bovine surrogate dam possessing an environment that is not suitable for achieving a successful embryo implantation and pregnancy.

13. The method of claim 1 wherein the lipidome analysis comprises mass spectroscopy.

14. The method of claim 13 wherein vaginal fluid sample data is delivered to a mass spectrometer from a handheld device.

15. The method of claim 13 wherein the mass spectrometry is conducted through a portable device.

16. A method for generating a lipidome database for identifying a bovine surrogate dam for embryo transfer, the method comprising: (a) collecting vaginal fluids individually from a population of bovine animals, wherein the vaginal fluids comprise a lipidome of each of the bovine animals; (b) performing lipidome analysis on the vaginal fluids to obtain a vaginal lipid profile, wherein an increased or decreased concentration in the vaginal lipid profile is predictive of a bovine surrogate dam possessing a suitable or unsuitable environment for achieving a successful embryo implantation and pregnancy; (c) delivering an embryo to at least one bovine animal; and (d) identifying an association of vaginal lipids in the vaginal lipidome profile with an increased probability of a successful embryo implantation and pregnancy following embryo delivery.

17. The method of claim 16 wherein the lipidome analysis comprises mass spectroscopy.

18. The method of claim 17 wherein vaginal fluid sample data is delivered to a mass spectrometer from a handheld device.

19. The method of claim 17 wherein the mass spectrometry is conducted through a portable device.

20. The method of claim 16 wherein the vaginal lipid profile comprises a vaginal phospholipid profile comprising a vaginal lipid level that is predictive of a candidate bovine surrogate dam possessing an environment that is suitable for achieving a successful embryo implantation and pregnancy.

21. The method of claim 16 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

22. A method for improving success rate of embryo transfer after in vitro fertilization in one or more bovine animals, the method comprising: (a) collecting vaginal fluid from each of the bovine animals, wherein the vaginal fluid comprises a lipidome of each the bovine animal; (b) performing lipidome analysis on the vaginal fluids to obtain a vaginal lipid profile, wherein an increase or decrease in the vaginal lipid profile is predictive of a bovine surrogate dam possessing a suitable environment or an unsuitable environment for achieving a successful embryo implantation and pregnancy following in vitro fertilization; and (c) administering an intervention to one or more of the bovine surrogate dams, wherein the intervention is effective to provide a recipient bovine surrogate dam with a lipidome profile associated with a high success rate of embryo transfer.

23. The method of claim 22 wherein the vaginal lipid profile comprises Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3)_FA 16:0.

24. The method of claim 22 wherein the lipidome analysis comprises mass spectroscopy.

25. The method of claim 24 wherein vaginal fluid sample data is delivered to a mass spectrometer from a handheld device.

26. The method of claim 24 wherein the mass spectrometry is conducted through a portable device.

27. The method of claim 22, wherein the intervention that is administered to the bovine surrogate dam is effective to provide a lipidome profile of the vaginal fluid exhibiting one or more lipid ratios and/or levels that are associated with high success of pregnancy after embryo transfer, including increased Cer(d18:1/16:0), SM(d16:1/24:0), SM(d18:1/16:0), PE(36:1), PCo(32:0), PC(38:2), 18:2 cholesteryl ester, 20:1 cholesteryl ester, TAG(50:4)_FA 16:1, and/or TAG(52:3_FA 16:0.

28. A diagnostic test kit for identifying a bovine surrogate dam having a high probability or a low probability of establishing a successful embryo implantation and pregnancy following embryo transfer via in vitro fertilization, the diagnostic test comprising: (a) one or more analytical tools for determining a vaginal lipid profile comprising vaginal lipid ratios and/or levels from the bovine animal; (b) a transmitter to communicate with a database of one or more vaginal lipid levels and/or ratios of the vaginal fluid; and (c) a device for comparing the vaginal lipid ratios and/or levels of the vaginal fluid from the surrogate dam with a database of vaginal lipid ratios identify a surrogate dam having a vaginal lipid ratio that is predictive of a high pregnancy success rate or a low pregnancy success rate after receiving an in vitro fertilization derived embryo.

29. The diagnostic test kit of claim 28 wherein the vaginal lipid profile comprises a vaginal lipid ratio for a first vaginal lipid and a second vaginal lipid.

30. The diagnostic test of claim 28 wherein the lipidome analysis comprises mass spectroscopy.

31. The diagnostic test kit of claim 30 wherein vaginal fluid sample data is delivered to a mass spectrometer from a handheld device.

32. The diagnostic test kit of claim 30 wherein the mass spectrometry is conducted through a portable device.

33. The method of claim 1, wherein the candidate bovine surrogate dam is a beef female.

34. The method of claim 1, wherein the candidate bovine surrogate dam is a dairy female.
